(19) United States
(12) Patent Application Publication
(10) Pub. No.: US 2026/0274912 A1
(43) Pub. Date: Sep. 17, 2026

(54) ENGINEERED IL-21 VARIANTS AND METHODS OF USE THEREOF

(71) Applicant: FBD BIOLOGICS LIMITED, Hong Kong (CN)

(72) Inventors: Chun-Yu LIN, Taipei City (TW); I-Yin CHEN, Taipei City (TW); I-CHun CHEN, Taipei City (TW); Yi-Shin LIAO, Taipei City (TW); Yi-Chun HSIEH, Taipei City (TW); Hsiang-Chun CHUANG, Taipei City (TW); Hsin-Tzu HSIEH, Taipei City (TW); Yi-Hsuan LU, Taipei City (TW); Chai-Yeh CHUANG, Taipei City (TW); Tsai-Kuei SHEN, Taipei City (TW); Yung-Chu LIN, Taipei City (TW); Yen-Cheng LI, Taipei City (TW); Yu-Chien CHEN, Taipei City (TW); Ying-Shao LU, Taipei City (TW); Chun-Hsien LEE, Taipei City (TW); Chiu-Yueh SHIH, Taipei City (TW); Chi-Ling TSENG, Taipei City (TW); Zong Sean JUO, Taipei City (TW)

(21) Appl. No.: 19/164,065

(22) PCT Filed: Mar. 19, 2024
(86) PCT No.: PCT/CN2024/082466

§ 371 Date: Sep. 10, 2025

Related U.S. Application Data
(60) Provisional application No. 63/453,399, filed on Mar. 20, 2023.

(51) Int. Cl. C07K 14/54; A61K 38/00; A61P 35/00

(52) U.S. Cl. CPC C07K 14/54; A61P 35/00; A61K 38/00; C07K 2319/21; C07K 2319/30; C07K 2319/31

(57) ABSTRACT

This disclosure relates to engineered IL-21 variants, and methods of use thereof. In some embodiments, the engineered IL-21 variants include a non-native disulfide bond.

### CROSS-REFERENCE TO RELATED APPLICATION

This disclosure claims priority to and benefit of U.S. Provisional Patent Application Ser. No. 63/453,399, filed Mar. 20, 2023, which is incorporated herein by reference in its entirety.

### SEQUENCE LISTING

This application contains a Sequence Listing that has been submitted electronically as an XML file named 52246-0012WO1_SL_ST26.xml. The XML file, created on Mar. 18, 2024, is 47,998 bytes in size. The material in the XML file is hereby incorporated by reference in its entirety.

### TECHNICAL FIELD

This disclosure relates to engineered IL-21 variants, and methods of use thereof.

### BACKGROUND

Cytokines belonging to the common γ chain (yc) family are central regulators of the development, proliferation, survival and differentiation of multiple cell lineages of the innate and adaptive immune system, and as such are of high interest for anticancer therapeutic applications.

Among them, IL-21 exerts potent antitumor effects due to its ability to induce and expand cytotoxic CD8+ T cells, NK cells and NKT cells, as well as to its capacity to suppress FOXP3 expression and the expansion of regulatory T cells (Tregs). In addition, IL-21 has been associated with clinical antineoplastic activity. However, at high concentrations, IL-21 can also lead to dose-limiting side effects including grade 3/4 granulocytopenia and liver toxicities. Moreover, by driving an inflammatory response sustained by IL-6 and IL-17, as it occurs during chronic colitis, IL-21 can also contribute to oncogenesis.

Recombinant IL-21 has been tested as an antitumor agent in various clinical trials. It showed promising antitumor activities and acceptable toxicity. However, the short half-life of IL-21 reduces its in vivo levels and requires frequent dosing, which limits its clinical application.

Thus, there is a need to develop cancer therapies targeting the IL-21 pathway with longer half-life, enhanced anti-tumor efficacy and limited toxicities.

### SUMMARY

This disclosure relates to engineered IL-21 variants, protein constructs (e.g., fusion proteins or protein complexes), and methods of use thereof. In some embodiments, the variants and protein constructs include a non-native disulfide bond formed by mutating 1, 2, 3, 4, 5, 6, or more than 6 residues (e.g., a pair of residues) of wild-type IL-21 to cysteines, without interfering the overall structure or residues critical for IL-21 receptor interaction. As a result, the variants or protein constructs can be more stable than wild-type IL-21. In some embodiments, the protein constructs showed enhanced thermostability (e.g., increased T_{agg} and Tₒₙₛₑₜ) and higher potency to relieve immune suppression (e.g., Treg-mediated T cell suppression) than wild-type IL-21. Therefore, the engineered IL-21 variants and protein constructs thereof described herein can be used for cancer treatment with minimal toxicity.

Also provided herein are methods for screening a cytokine (e.g., IL-21) with increased stability and/or anti-tumor efficacy.

In one aspect, the disclosure is related to an engineered IL-21 polypeptide, in some embodiments, the engineered IL-21 polypeptide comprises a non-native disulfide bond. In some embodiments, the engineered IL-21 polypeptide comprises an amino acid sequence that is at least 80% identical to SEQ ID NO: 2. In some embodiments, the engineered IL-21 polypeptide can bind to a complex formed by human IL-21 receptor (IL-21R) and common cytokine γ chain (γc).

In some embodiments, the engineered IL-21 polypeptide comprises one or more of the following: (a) the amino acid that corresponds to position 8 of SEQ ID NO: 2 is cysteine (C); (b) the amino acid that corresponds to position 19 of SEQ ID NO: 2 is C; (c) the amino acid that corresponds to position 29 of SEQ ID NO: 2 is C; (d) the amino acid that corresponds to position 31 of SEQ ID NO: 2 is C; (e) the amino acid that corresponds to position 33 of SEQ ID NO: 2 is C; (f) the amino acid that corresponds to position 36 of SEQ ID NO: 2 is C; (g) the amino acid that corresponds to position 39 of SEQ ID NO: 2 is C; and (h) the amino acid that corresponds to position 56 of SEQ ID NO: 2 is C.

In some embodiments, the engineered IL-21 polypeptide comprises one or more of the amino acid that corresponds to position 61 of SEQ ID NO: 2 is C; the following: (a) (b) the amino acid that corresponds to position 62 of SEQ ID NO: 2 is C; (c) the amino acid that corresponds to position 63 of SEQ ID NO: 2 is C; (d) the amino acid that corresponds to position 80 of SEQ ID NO: 2 is C; (e) the amino acid that corresponds to position 86 of SEQ ID NO: 2 is C; (f) the amino acid that corresponds to position 105 of SEQ ID NO: 2 is C; (g) the amino acid that corresponds to position 106 of SEQ ID NO: 2 is C; (h) the amino acid that corresponds to position 107 of SEQ ID NO: 2 is C; (i) the amino acid that corresponds to position 110 of SEQ ID NO: 2 is C; (j) the amino acid that corresponds to position 112 of SEQ ID NO: 2 is C; and (k) the amino acid that corresponds to position 117 of SEQ ID NO: 2 is C. In some embodiments, the engineered IL-21 polypeptide comprises one or more of the following: (a) the amino acid that corresponds to position 8 of SEQ ID NO: 2 is C, and the amino acid that corresponds to position 86 of SEQ ID NO: 2 is C; (b) the amino acid that corresponds to position 19 of SEQ ID NO: 2 is C, and the amino acid that corresponds to position 117 of SEQ ID NO: 2 is C; (c) the amino acid that corresponds to position 29 of SEQ ID NO: 2 is C, and the amino acid that corresponds to position 110 of SEQ ID NO: 2 is C; (d) the amino acid that corresponds to position 31 of SEQ ID NO: 2 is C, and the amino acid that corresponds to position 62 of SEQ ID NO: 2 is C; (e) the amino acid that corresponds to position 31 of SEQ ID NO: 2 is C, and the amino acid that corresponds to position 63 of SEQ ID NO: 2 is C; (f) the amino acid that corresponds to position 33 of SEQ ID NO: 2 is C, and the amino acid that corresponds to position 61 of SEQ ID NO: 2 is C; (g) the amino acid that corresponds to position 33 of SEQ ID NO: 2 is C, and the amino acid that corresponds to position 63 of SEQ ID NO: 2 is C; (h) the amino acid that corresponds to position 36 of SEQ ID NO: 2 is C, and the amino acid that corresponds to position 106 of SEQ ID NO: 2 is C; (i) the amino acid that corresponds to position 36 of SEQ ID NO: 2 is C, and the amino acid that corresponds to position 107 of SEQ ID NO: 2 is C; (j) the amino acid that corresponds to position 39 of SEQ ID NO: 2 is C, and the amino acid that corresponds to position 105 of SEQ ID NO: 2 is C; (k) the amino acid that corresponds to position 39 of SEQ ID NO: 2 is C, and the amino acid that corresponds to position 107 of SEQ ID NO: 2 is C; (1) the amino acid that corresponds to position 39 of SEQ ID NO: 2 is C, and the amino acid that corresponds to position 112 of SEQ ID NO: 2 is C; and (m) the amino acid that corresponds to position 56 of SEQ ID NO: 2 is C, and the amino acid that corresponds to position 80 of SEQ ID NO: 2 is C.

In some embodiments, the engineered IL-21 polypeptide comprises one or more of the following: (a) the amino acid that corresponds to position 12 of SEQ ID NO: 2 is M; (b) the amino acid that corresponds to position 16 of SEQ ID NO: 2 is R; (c) the amino acid that corresponds to position 19 of SEQ ID NO: 2 is I; (d) the amino acid that corresponds to position 23 of SEQ ID NO: 2 is D; (e) the amino acid that corresponds to position 105 of SEQ ID NO: 2 is E; (f) the amino acid that corresponds to position 114 of SEQ ID NO: 2 is E; (g) the amino acid that corresponds to position 118 of SEQ ID NO: 2 is S; (h) the amino acid that corresponds to position 121 of SEQ ID NO: 2 is Q; (i) the amino acid that corresponds to position 122 of SEQ ID NO: 2 is K; (j) the amino acid that corresponds to position 124 of SEQ ID NO: 2 is I; (k) the amino acid that corresponds to position 125 of SEQ ID NO: 2 is H; and (1) the amino acid that corresponds to position 128 of SEQ ID NO: 2 is L.

In some embodiments, the engineered IL-21 polypeptide described herein comprises an amino acid sequence that is at least 85%, 90%, 95%, 96%, 97%, 98%, 99%, or 100% identical to SEQ ID NO: 3, 4, 5, 6, 7, 8, 9, 10, 11, 12, 13, 14, or 15. In some embodiments, the engineered IL-21 polypeptide comprises an amino acid sequence that is at least 90% identical to SEQ ID NO: 3. In some embodiments, the engineered IL-21 polypeptide comprises an amino acid sequence that is at least 90% identical to SEQ ID NO: 5. In some embodiments, the engineered IL-21 polypeptide comprises an amino acid sequence that is at least 90% identical to SEQ ID NO: 9. In some embodiments, the engineered IL-21 polypeptide comprises an amino acid sequence that is at least 90% identical to SEQ ID NO: 10.

In some embodiments, the engineered IL-21 polypeptide can induce proliferation of immune cells (e.g., T cells or NK cells). In some embodiments, the engineered IL-21 polypeptide can induce STAT-3 phosphorylation.

In one aspect, the disclosure is related to a fusion protein comprising the engineered IL-21 polypeptide described herein. In some embodiments, the fusion protein described herein further comprises a human serum albumin (HSA). In some embodiments, the HSA comprises an amino acid sequence that is at least 85%, 90%, 95%, 96%, 97%, 98%, 99%, or 100% identical to SEQ ID NO: 26. In some embodiments, the engineered IL-21 polypeptide is linked to the C-terminus to the HSA via a linker peptide. In some embodiments, the linker peptide comprises an amino acid sequence that is at least 85%, 90%, 95%, 96%, 97%, 98%, 99%, or 100% identical to SEQ ID NO: 25. In some embodiments, the fusion protein further comprises a His-tag, optionally at the N-terminus. In some embodiments, the fusion protein comprises an amino acid sequence that is at least 85%, 90%, 95%, 96%, 97%, 98%, 99%, or 100% identical to SEQ ID NO: 17, 18, 19, 20, or 21.

In one aspect, the disclosure is related to a fusion protein comprising, optionally from N-terminus to C-terminus: (a) optionally a His-tag; (b) a HSA; (c) a linker peptide, and (d) an engineered IL-21 polypeptide. In some embodiments, the His-tag comprises an amino acid sequence that is at least 85%, 90%, 95%, 96%, 97%, 98%, 99%, or 100% identical to SEQ ID NO: 22. In some embodiments, the HSA comprises an amino acid sequence that is at least 85%, 90%, 95%, 96%, 97%, 98%, 99%, or 100% identical to SEQ ID NO: 26. In some embodiments, the linker peptide comprises an amino acid sequence that is at least 85%, 90%, 95%, 96%, 97%, 98%, 99%, or 100% identical to SEQ ID NO: 25. In some embodiments, the engineered IL-21 polypeptide comprises an amino acid sequence that is at least 85%, 90%, 95%, 96%, 97%, 98%, 99%, or 100% identical to SEQ ID NO: 2, 3, 4, 5, 6, 7, 8, 9, 10, 11, 12, 13, 14, or 15.

In some embodiments, the fusion protein described herein further comprises an Fc region.

In some embodiments, the fusion protein can relieve Treg-mediated T cell suppression. In some embodiments, the fusion protein can induce primary NK cell cytotoxicity.

In one aspect, the disclosure is related to a protein complex comprising: (a) a first polypeptide comprising from N-terminus to C-terminus: an optional first hinge region, a first Fc region, an optional linker peptide, and the engineered IL-21 polypeptide described herein; and (b) a second polypeptide comprising from N-terminus to C-terminus, an optional second hinge region, a second Fc region. In some embodiments, the first hinge region, the first Fc region, the second hinge region, and/or the second Fc region are derived from human IgG4. In some embodiments, the first Fc region and/or the second Fc region comprise one or more knob-into-hole (KIH) mutations. In some embodiments, the first Fc region comprises a sequence that is at least 80%, 90%, 95%, or 100% to SEQ ID NO: 33, and the second Fc region comprises a sequence that is at least 80%, 90%, 95%, or 100% to SEQ ID NO: 32. In some embodiments, the first hinge region and/or the second hinge region comprise a sequence that is at least 80%, 90%, 95%, or 100% to SEQ ID NO: 31. In some embodiments, the linker peptide comprises a sequence that is at least 80%, 90%, 95%, or 100% to SEQ ID NO: 34. In some embodiments, protein complex described herein comprises one of the followings: (1) the first polypeptide comprises a sequence that is at least 80%, 90%, 95%, or 100% to SEQ ID NO: 35, and the first polypeptide comprises a sequence that is at least 80%, 90%, 95%, or 100% to SEQ ID NO: 29; (2) the first polypeptide comprises a sequence that is at least 80%, 90%, 95%, or 100% to SEQ ID NO: 36, and the first polypeptide comprises a sequence that is at least 80%, 90%, 95%, or 100% to SEQ ID NO: 29; (3) the first polypeptide comprises a sequence that is at least 80%, 90%, 95%, or 100% to SEQ ID NO: 37, and the first polypeptide comprises a sequence that is at least 80%, 90%, 95%, or 100% to SEQ ID NO: 29; (4) the first polypeptide comprises a sequence that is at least 80%, 90%, 95%, or 100% to SEQ ID NO: 38, and the first polypeptide comprises a sequence that is at least 80%, 90%, 95%, or 100% to SEQ ID NO: 29; or (5) the first polypeptide comprises a sequence that is at least 80%, 90%, 95%, or 100% to SEQ ID NO: 39, and the first polypeptide comprises a sequence that is at least 80%, 90%, 95%, or 100% to SEQ ID NO: 29.

In some embodiments, the protein complex can induce STAT-3 phosphorylation and/or relieve Treg-mediated T cell suppression. In some embodiments, the protein complex can induce proliferation of immune cells (e.g., T cells or NK cells), and/or induce primary NK cell cytotoxicity.

In one aspect, the disclosure is related to a pharmaceutical composition comprising the engineered IL-21 polypeptide, the fusion protein, or the protein complex described herein; and a pharmaceutically acceptable carrier.

In one aspect, the disclosure is related to a nucleic acid encoding the engineered IL-21 polypeptide, the fusion protein, or the protein complex described herein. In one aspect, the disclosure is related to a vector comprising the nucleic acid described herein.

In one aspect, the disclosure is related to a cell comprising the nucleic acid or the vector described herein. In some embodiments, the cell is a Expi293 cell or CHO-S cell.

In one aspect, the disclosure is related to a method of producing an engineered IL-21 polypeptide or a fusion protein comprising the engineered IL-21 polypeptide, the method comprising (a) culturing the cell described herein under conditions sufficient for the cell to produce the engineered IL-21 polypeptide or the fusion protein; and (b) collecting the engineered IL-21 polypeptide, the fusion protein, or the protein complex produced by the cell.

In one aspect, the disclosure is related to a method of treating a subject having cancer, the method comprising administering a therapeutically effective amount of a composition comprising the engineered IL-21 polypeptide, the fusion protein, or the protein complex described herein, to the subject. In some embodiments, the subject has a solid tumor or a hematologic cancer. In some embodiments, the cancer is melanoma, renal cell carcinoma (RCC), lymphoma, esophageal adenocarcinoma, lung cancer, hepatocellular carcinoma, cholangiocarcinoma, gallbladder cancer, gastric cancer, pancreatic cancer, colorectal cancer, endometrial carcinoma, ovarian cancer, bladder cancer, or prostate cancer.

In one aspect, the disclosure is related to a method of decreasing the rate of tumor growth, the method comprising contacting a tumor cell with an effective amount of a composition comprising the engineered IL-21 polypeptide, the fusion protein, or the protein complex described herein.

In one aspect, the disclosure is related to a method of killing a tumor cell, the method comprising contacting a tumor cell with an effective amount of a composition comprising the engineered IL-21 polypeptide, the fusion protein, or the protein complex described herein.

In one aspect, the disclosure is related to a method of improving the stability of a protein (e.g., cytokine), comprising (a) providing a 3D structure of the protein (e.g., cytokine), (b) measuring distance of the Cₐₗₚₕₐ atoms of one or more of amino acid residues in the 3D structure; and (c) selecting two amino acid residues from the one or more amino acid residues, in some embodiments, the Cₐₗₚₕₐ atoms of the two selected amino acid residues are within 3-7 angstroms (e.g., 4.5-6.5 angstroms). In some embodiments, the method described herein further comprises expressing a protein variant (e.g., a cytokine variant), in some embodiments, the protein variant comprises a non-native disulfide bond formed by mutating the two selected amino acid residues to cysteines. In some embodiments, the mutating the two selected amino acid residues to cysteines does not substantially change the 3D structure of the protein (e.g., cytokine).

In one aspect, the disclosure is related to a method of screening a cytokine variant with an improved anti-tumor efficacy, comprising (a) providing a 3D structure of the cytokine, (b) measuring distance of the Cₐₗₚₕₐ atoms of one or more of amino acid residues in the 3D structure; and (c) selecting two amino acid residues from the one or more amino acid residues, in some embodiments, the Cₐₗₚₕₐ atoms of the two selected amino acid residues are within 3-7 angstroms (e.g., 4.5-6.5 angstroms). In some embodiments, the method described herein further comprises (d) expressing a cytokine variant, in some embodiments, the variant comprises a non-native disulfide bond formed by mutating the two selected amino acid residues to cysteines; (e) administering the cytokine variant to a tumor-bearing animal; and (f) determining tumor growth (e.g., by measuring tumor volume) in the tumor-bearing animal.

In some embodiments, the protein or cytokine described herein is IL-21 (e.g., human IL-21).

In some embodiments, the protein or cytokine has no more than 90, 100, 110, 120, 130, 140, 150, 160, 170, 180, 190, or 200 amino acid residues.

As used herein, the term “non-native disulfide bond” refers to a disulfide bond that does not naturally exist in a wild-type protein. In some embodiments, the non-native disulfide bond is formed by two cysteine residues, wherein at least one of them is a mutation. In some embodiments, two of them are mutations. In some embodiments, at least one or two cysteine residues are introduced by insertion. In some embodiments, a deletion changes the distance between two existing cysteine residues, which then forms a disulfide bond that does not exist in a wild-type protein.

As used herein, the term “engineered IL-21 polypeptide” refers to a polypeptide derived from a wild-type IL-21 polypeptide or a portion thereof, optionally with one or more mutations (e.g., insertions, deletions, or substitutions). In some embodiments, the engineered IL-21 polypeptide comprises or consists of an amino acid sequence corresponding to amino acids 25-162 of human IL-21 (SEQ ID NO: 1). In some embodiments, the engineered IL-21 polypeptide has one or more (e.g., 1, 2, 3, 4, 5, 6, 7, 8, 9, or 10) mutations (e.g., amino acids are substituted by cysteine).

As used herein, the term “protein construct” refers to a complex having one or more polypeptides. In some embodiments, the protein construct is a fusion protein, e.g., a fusion protein including a HSA and an engineered IL-21 polypeptide (e.g., any of the engineered IL-21 polypeptides described herein). In some embodiments, the protein construct has two or more polypeptides, wherein the polypeptides can associate with each other, forming a dimer or a multimer (e.g., a trimer). In some embodiments, the protein construct is a heterodimeric Fc-fused IL-21 (e.g., any of the heterodimeric Fc-fused IL-21 or its variants described herein).

As used herein, the term “cancer” refers to cells having the capacity for uncontrolled autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof. A hematologic cancer is a cancer that begins in blood-forming tissue, such as the bone marrow, or in the cells of the immune system. Examples of hematologic cancer include e.g., leukemia, lymphoma, and multiple myeloma etc.

As used herein, the terms “subject” and “patient” are used interchangeably throughout the specification and describe an animal, human or non-human, to whom treatment according to the methods of the present invention is provided. Veterinary and non-veterinary applications are contemplated in the present disclosure. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a table summarizing the purification profile of IL-21 variants in Expi293 and CHO-S cells. “H8” stands for 8×His tag.

FIG. 2A shows the potency of IL-21 variants on induction of STAT-3 phosphorylation.

FIG. 2B shows the potency of IL-21 variants on induction of T cell proliferation.

FIG. 3A shows SDS-PAGE results of selected HSA-fused IL-21 variants. NR: non-reducing. R: reducing.

FIG. 3B is a table summarizing HPLC-SEC analysis results of HSA-fused IL-21 variants.

FIG. 3C is a table summarizing DLS/SLS analysis results of HSA-fused IL-21 variants.

FIG. 4A shows the potency of HSA-fused IL-21 variants on induction of STAT-3 phosphorylation.

FIG. 4B shows the potency of HSA-fused IL-21 variants on induction of T cell proliferation.

FIG. 5 shows the potency of HSA-fused IL-21 variants on induction of NK-92 cell proliferation.

FIG. 6 shows the potency of HSA-fused IL-21 variants on relieving Treg-mediated T cell suppression.

FIG. 7 shows the potency of HSA-fused IL-21 variants on induction of primary NK cell cytotoxicity.

FIG. 8A is a table showing treatment plan and dosing schedule of HSA-fused IL-21 and its variants in a CT26 bearing BLAB/c mouse model.

FIG. 8B shows the average tumor growth curves of CT26-bearing BALB/c mice that were treated with HSA-fused IL-21 and its variants. After grouping (Day 0), the mice were injected with HSA-fused IL-21 and its variants on Day 4, Day 7, Day 11, Day 14, and Day 18 (indicated by arrows).

FIGS. 8C-8F show individual tumor growth curves of CT26-bearing BALB/c mice that were treated with vehicle (FIG. 8C), HSA-IL21-WT (FIG. 8D), HSA-IL21-C01 (FIG. 8E), and HSA-IL21-C08 (FIG. 8F), respectively.

FIG. 8G shows individual tumor volume of each CT26-bearing BALB/c mouse that were treated with HSA-fused IL-21 and its variants on Day 21 (or Day 25 after inoculation).

FIG. 8H shows average body weight change of CT26-bearing BALB/c mice that were treated with HSA-fused IL-21 and its variants.

FIG. 8I is a table showing the tumor volume (TV) on Day 21 (or Day 25 after inoculation), the average percentage of tumor growth inhibition (TGI %) and corresponding p value of CT26-bearing BALB/c mice that were treated with HSA-fused IL-21 and its variants. The P values were determined using one-way ANONA analysis.

FIG. 9A shows the potency of HSA-fused IL-21 variants on relieving Treg-mediated T cell suppression. hSIRPα-Fc-mt10 (SEQ ID NO: 42) was used as a negative control.

FIG. 9B shows the percentage of live CD8⁺CFSE^{low} cells after incubation of activated CD8⁺ T cells (Teff) and Treg cells that were pretreated with 30 nM HSA-IL21-WT (“WT”), HSA-IL21-C07 (“C07”), or HSA-IL21-C08 (“C08”).

FIG. 10 shows the potency of HSA-fused IL-21 variants on induction of primary NK cell cytotoxicity.

FIG. 11A shows treatment plan and dosing schedule of HSA-fused IL-21 and its variants in a CT26 bearing BLAB/c mouse model.

FIG. 11B shows the average tumor growth curves of CT26-bearing BALB/c mice that were treated with HSA-fused IL-21 and its variants. After grouping (Day 0), the mice were injected with HSA-fused IL-21 and its variants on Day 4, Day 8, Day 11, Day 14, Day 18, and Day 21 (indicated by arrows).

FIGS. 11C-11F show individual tumor growth curves of CT26-bearing BALB/c mice that were treated with vehicle (FIG. 11C), HSA-IL21-WT (FIG. 11D), HSA-IL21-C01 (FIG. 11E), and HSA-IL21-C08 (FIG. 11F), respectively.

FIG. 11G shows average body weight change of CT26-bearing BALB/c mice that were treated with HSA-fused IL-21 and its variants.

FIG. 11H shows survival curves of CT26-bearing BALB/c mice that were treated with HSA-fused IL-21 and its variants.

FIGS. 11I-11K show individual tumor growth curves of CT26-bearing BALB/c mice that were rechallenged by inoculating CT26 cells. 6 mice injected with vehicle were used as controls for tumor rechallenge (FIG. 11I). 10 mice previously inoculated with CT26 and then treated with HSA-IL21-C01 with a tumor size below 50 mm³ (n=10) were selected for tumor rechallenge (FIG. 11J). 11 mice previously inoculated with CT26 and then treated with HSA-IL21-C08 with a tumor size below 50 mm³ (n=11) were selected for tumor rechallenge (FIG. 11K).

FIG. 12 shows the potency of heterodimeric Fc-fused IL-21 variants on induction of STAT-3 phosphorylation. Human recombinant IFN-gamma protein (BioLegend, Cat #: 570208) was used as a negative control.

FIG. 13A shows the potency of heterodimeric Fc-fused IL-21 variants on induction of T cell proliferation. hSIRPα-Fc-mt10 (SEQ ID NO: 42) was used as a negative control.

FIG. 13B shows the potency of heterodimeric Fc-fused IL-21 variants on induction of NK-92 cell proliferation. hSIRPα-Fc-mt10 (SEQ ID NO: 42) was used as a negative control.

FIG. 14 shows the potency of heterodimeric Fc-fused IL-21 variants on induction of primary NK cell cytotoxicity.

FIG. 15A is a table showing treatment plan and dosing schedule of heterodimeric Fc-fused IL-21 and its variants in a CT26 bearing BLAB/c mouse model.

FIGS. 15B-15G show individual tumor growth curves of CT26-bearing BALB/c mice that were treated with vehicle (FIG. 15B), heterodimeric Fc-IL21-WT (FIG. 15C), heterodimeric Fc-IL21-C01 (FIG. 15D), heterodimeric Fc-IL21-C03 (FIG. 15E), heterodimeric Fc-IL21-C07 (FIG. 15F), and heterodimeric Fc-IL21-C08 (FIG. 15G), respectively. After grouping (Day 0), the mice were injected with heterodimeric Fc-fused IL-21 and its variants on Day 4, Day 7, Day 10, Day 13, Day 17, and Day 20 (indicated by arrows).

FIG. 15H shows average body weight change of CT26-bearing BALB/c mice that were treated with heterodimeric Fc-fused IL-21 and its variants.

FIG. 16 lists amino acid sequences discussed in the disclosure.

### DETAILED DESCRIPTION

Interleukin-21 (also known as IL-21, IL21, Za11, or CVID11) is a pleiotropic cytokine that is composed of four α-helical bundles and produced primarily by natural killer T (NKT) cells, T follicular helper (T_{FH}) cells and TH17 cells, with lower levels of production by numerous other populations of lympho-haematopoietic cells. IL-21 signals via heterodimers of the IL-21 receptor (IL-21R) and the common cytokine receptor γ-chain, γc (encoded by IL2RG). IL-21 signals through its receptor complex composed of the private chain IL-21Rα and the common chain, γC, the latter of which is shared by five other cytokines: IL-2, IL-4, IL-7, IL-9, and IL-15. Together these cytokines constitute the so-called γC family of cytokines. Despite the rather limited sequence homology (on average 15% sequence identity), these γC cytokines share a highly conserved overall four-helix bundle topology.

Functional IL-21R is broadly expressed on lympho-haematopoietic populations, including on myeloid cells. Correspondingly, IL-21 exerts its effects on a broad range of cell types. Given the breadth of immunomodulatory targets and the pleiotropic actions of IL-21, IL-21 and IL-21R are attractive targets for therapeutic manipulation; indeed, antibodies against IL-21 and IL-21R as well as IL-21 antagonists have been developed.

IL-21 signals via the Janus kinase (JAK)-signal transducer and activator of transcription (STAT) signaling pathway, the mitogen-activated protein kinase (MAPK) signaling pathway and the phosphoinositide 3-kinase (PI3K)-AKT signaling pathway. Like other γc family cytokines, IL-21 activates JAK1 and JAK3. Interestingly, in T cells, IL-21 activates STAT3 more potently and in a more sustained fashion than STAT1, STAT5A and STAT5B8. In studies of the regulation of mouse B lymphocyte-induced maturation protein 1 (BLIMP1; a transcription factor encoded by the Prdml gene), an IL-21 response element was shown to be composed of a bipartite element that binds both interferon regulatory factor 4 (IRF4) and STAT3. Unexpectedly, when analyzed by chromatin immunoprecipitation linked to next-generation sequencing (ChIP-seq), such bipartite response elements were found in a genome-wide fashion and were globally involved in the regulation of many IL-21-responsive genes.

A detailed description of IL-21 and its function can be found, e.g., in Spolski, R., et al. “Interleukin-21: a double-edged sword with therapeutic potential.” Nature Reviews Drug Discovery 13.5 (2014): 379-395; Kang, L., et al. “Rational design of interleukin-21 antagonist through selective elimination of the γC binding epitope.” Journal of Biological Chemistry 285.16 (2010): 12223-12231; Croce, M., et al. “IL-21: a pleiotropic cytokine with potential applications in oncology.” Journal of Immunology Research 2015 (2015); and Stolfi, C., et al. “Interleukin-21 in cancer immunotherapy: Friend or foe?.” Oncoimmunology 1.3 (2012): 351-354; each of which is incorporated by reference in its entirety.

The present disclosure provides engineered IL-21 variants having at least one non-native disulfide bond. In some embodiments, two amino acid residues in a wild-type human IL-21 (e.g., SEQ ID NO: 2) are selectively mutated to cysteines, which can form the non-native disulfide bond. In some embodiments, the mutations do not substantially change the overall structure of IL-21, e.g., the relative position of the four alpha helices of IL-21. In some embodiments, the non-native disulfide bond formed by the cysteine mutations can stabilize IL-21. In some embodiments, the engineered IL-21 variants described herein can bind to the IL-21R/yc complex, and induce downstream signaling pathways (e.g., STAT3 phosphorylation) and/or immune cell (e.g., T cell or NK cell) proliferation. In some embodiments, the engineered IL-21 variants comprises or consists of any of the engineered IL-21 polypeptides described herein.

Also provided herein are protein constructs (e.g., fusion proteins) that further include a human serum albumin (HSA) that is fused to the engineered IL-21 variants described herein. In some embodiments, the HSA can stabilize the engineered IL-21 variants. In some embodiments, the protein constructs can promote immune response (e.g., relieve Treg-mediated T cell suppression and/or induce primary NK cell cytotoxicity).

Also provided herein are protein constructs (e.g., protein complexes) that an engineered IL-21 variant (e.g., any of the engineered IL-21 variants described herein) is linked to the C-terminal end of a human IgG4 Fc, forming a heterodimer (e.g., any of the heterodimeric Fc-fused IL-21 variants described herein). In some embodiments, the protein complexes can promote immune response (e.g., induce IL-21-STAT3 signaling, induce immune cell proliferation, and/or induce primary NK cell cytotoxicity).

The disclosure also provides methods of screening cytokine (e.g., IL-21) variants having a higher stability and/or improved anti-tumor efficacy.

### Engineered IL-21 Variants

IL-21 is expressed in activated human CD4+ T cells but not in most other tissues. In addition, IL-21 expression is up-regulated in Th2 and Th17 subsets of T helper cells, as well as T follicular cells. In fact, it was shown that IL-21 can be used to identify peripheral T follicular helper cells. Furthermore, IL-21 is expressed in NK T cells regulating the function of these cells.

Human IL-21 includes, from N-terminus to C-terminus, a signal peptide, and a soluble chain. According to the UniProt Database (UniProt ID: Q9HBE4), the signal peptide of human IL-21 corresponds to amino acids 1-24 of SEQ ID NO: 1, and the soluble chain of human IL-21 corresponds to amino acids 25-162 of SEQ ID NO: 1. The soluble chain of human IL-21 is also shown as SEQ ID NO: 2. Although the average sequence homology between the γc cytokines IL-21, IL-2, and IL-4 is only about 17%, the topology of the four-helical bundle, which forms the core structure of these γc cytokines, is highly conserved. Specifically, the first alpha helix corresponds to M12 to N30 of SEQ ID NO: 2, the second alpha helix corresponds to E48 to A58 of SEQ ID NO: 2, the third alpha helix corresponds to N67 to K78 of SEQ ID NO: 2, and the fourth alpha helix corresponds to P109 to L128 of SEQ ID NO: 2. According to the model of the IL-21/IL-21R/yc complex, the following residues of IL-21 were identified as positions potentially critical for IL-21R or γc binding: amino acids corresponding to positions 12, 16, 19, 23, 105, 114, 118, 121, 122, 124, 125, and 128 of SEQ ID NO: 2. Specifically, these residues are M12, R16, 119, D23, E105, E114, S118, Q121, K122, 1124, H125, and L128 in SEQ ID NO: 2. Details can be found, e.g., in Kang, L., et al. “Rational design of interleukin-21 antagonist through selective elimination of the γC binding epitope.” Journal of Biological Chemistry 285.16 (2010): 12223-12231, which is incorporated herein by reference in its entirety.

Based on the 3D structure of human IL-21, distance of the Cₐₗₚₕₐ atoms of one or more amino acid residues (e.g., those in close proximity) in the 3D structure can be determined. Two residues whose Cₐₗₚₕₐ atoms are within 3-7 angstroms (e.g., 4.5-6.5 angstroms) can be selectively mutated to cysteines, without interfering the overall structure (e.g., the core structure formed by the four-helical bundle) of IL-21. It is contemplated that the newly introduced cysteines can form a disulfide bond non-native to the wild-type IL-21, which can stabilize IL-21 and/or improve the functional potencies of IL-21.

Thus, in one aspect, the engineered IL-21 variant (e.g., engineered IL-21 polypeptide) includes a first cysteine mutation and a second cysteine mutation, such that the two cysteines can form a non-native disulfide bond. In some embodiments, the first cysteine mutation occurs at an amino acid residue that corresponds to position 8, 19, 29, 31, 33, 36, 39, or 56 of SEQ ID NO: 2. In some embodiments, the second cysteine mutation occurs at an amino acid residue that corresponds to position 61, 62, 63, 80, 86, 105, 106, 107, 110, 112, or 117 of SEQ ID NO: 2. In some embodiments, the Cₐₗₚₕₐ atoms of the two selected amino acid residues are within 5 Å, 4.9 Å, 4.8 Å, 4.7 Å, 4.6 Å, 4.5 Å, 4.4 Å, 4.3 Å, 4.2 Å, 4.1 Å, 4 Å, 3.9 Å, 3.8 Å, 3.7 Å, 3.6 Å, 3.5 Å, 3.4 Å, 3.3 Å, 3.2 Å, 3.1 Å, 3 Å, 2.9 Å, 2.8 Å, 2.7 Å, 2.6 Å, 2.5 Å, 2.4 Å, 2.3 Å, 2.2 Å, 2.1 Å, or 2 Å. In some embodiments, any of the first cysteine mutations can be paired with any of the second cysteine mutations described herein, when distance of the Cₐₗₚₕₐ atoms of the two selected amino acid residues is within the range described above. In some embodiments, the non-native disulfide bond can further stabilize the overall structure of IL-21, e.g., the relative position and angles of the four-helical bundle in IL-21 are maintained.

In some embodiments, provided herein is an engineered IL-21 polypeptide IL21-C01, which includes a first cysteine residue at a position corresponding to position 8 (e.g., Q8) of SEQ ID NO: 2, and a second cysteine residue at a position corresponding to position 86 (T86) of SEQ ID NO: 2. The sequence of IL21-C01 is set forth in SEQ ID NO: 3.

In some embodiments, provided herein is an engineered IL-21 polypeptide IL21-C02, which includes a first cysteine residue at a position corresponding to position 19 (e.g., 119) of SEQ ID NO: 2, and a second cysteine residue at a position corresponding to position 117 (K117) of SEQ ID NO: 2. The sequence of IL21-C02 is set forth in SEQ ID NO: 4.

In some embodiments, provided herein is an engineered IL-21 polypeptide IL21-C03, which includes a first cysteine residue at a position corresponding to position 19 (e.g., V29) of SEQ ID NO: 2, and a second cysteine residue at a position corresponding to position 110 (K110) of SEQ ID NO: 2. The sequence of IL21-C03 is set forth in SEQ ID NO: 5.

In some embodiments, provided herein is an engineered IL-21 polypeptide IL21-C04, which includes a first cysteine residue at a position corresponding to position 31 (e.g., D31) of SEQ ID NO: 2, and a second cysteine residue at a position corresponding to position 62 (S62) of SEQ ID NO: 2. The sequence of IL21-C04 is set forth in SEQ ID NO: 6.

In some embodiments, provided herein is an engineered IL-21 polypeptide IL21-C05, which includes a first cysteine residue at a position corresponding to position 31 (e.g., D31) of SEQ ID NO: 2, and a second cysteine residue at a position corresponding to position 63 (A63) of SEQ ID NO: 2. The sequence of IL21-C05 is set forth in SEQ ID NO: 7.

In some embodiments, provided herein is an engineered IL-21 polypeptide IL21-C06, which includes a first cysteine residue at a position corresponding to position 33 (e.g., V33) of SEQ ID NO: 2, and a second cysteine residue at a position corresponding to position 61 (K61) of SEQ ID NO: 2. The sequence of IL21-C06 is set forth in SEQ ID NO: 8.

In some embodiments, provided herein is an engineered IL-21 polypeptide IL21-C07, which includes a first cysteine residue at a position corresponding to position 33 (e.g., V33) of SEQ ID NO: 2, and a second cysteine residue at a position corresponding to position 63 (A63) of SEQ ID NO: 2. The sequence of IL21-C07 is set forth in SEQ ID NO: 9.

In some embodiments, provided herein is an engineered IL-21 polypeptide IL21-C08, which includes a first cysteine residue at a position corresponding to position 36 (e.g., F36) of SEQ ID NO: 2, and a second cysteine residue at a position corresponding to position 106 (K106) of SEQ ID NO: 2. The sequence of IL21-C08 is set forth in SEQ ID NO: 10.

In some embodiments, provided herein is an engineered IL-21 polypeptide IL21-C09, which includes a first cysteine residue at a position corresponding to position 36 (e.g., F36) of SEQ ID NO: 2, and a second cysteine residue at a position corresponding to position 107 (K107) of SEQ ID NO: 2. The sequence of IL21-C09 is set forth in SEQ ID NO: 11.

In some embodiments, provided herein is an engineered IL-21 polypeptide IL21-C10, which includes a first cysteine residue at a position corresponding to position 39 (e.g., A39) of SEQ ID NO: 2, and a second cysteine residue at a position corresponding to position 105 (E105) of SEQ ID NO: 2. The sequence of IL21-C10 is set forth in SEQ ID NO: 12.

In some embodiments, provided herein is an engineered IL-21 polypeptide IL21-C11, which includes a first cysteine residue at a position corresponding to position 39 (e.g., A39) of SEQ ID NO: 2, and a second cysteine residue at a position corresponding to position 107 (K107) of SEQ ID NO: 2. The sequence of IL21-C11 is set forth in SEQ ID NO: 13.

In some embodiments, provided herein is an engineered IL-21 polypeptide IL21-C12, which includes a first cysteine residue at a position corresponding to position 39 (e.g., A39) of SEQ ID NO: 2, and a second cysteine residue at a position corresponding to position 112 (F112) of SEQ ID NO: 2. The sequence of IL21-C12 is set forth in SEQ ID NO: 14.

In some embodiments, provided herein is an engineered IL-21 polypeptide IL21-C13, which includes a first cysteine residue at a position corresponding to position 56 (e.g., Q56) of SEQ ID NO: 2, and a second cysteine residue at a position corresponding to position 80 (K80) of SEQ ID NO: 2. The sequence of IL21-C13 is set forth in SEQ ID NO: 15.

In some embodiments, provided herein is an engineered IL-21 polypeptide comprising one or more of the following: (a) the amino acid that corresponds to Q8 of SEQ ID NO: 2 is C, and the amino acid that corresponds to T86 of SEQ ID NO: 2 is C; (b) the amino acid that corresponds to 119 of SEQ ID NO: 2 is C, and the amino acid that corresponds to K117 of SEQ ID NO: 2 is C; (c) the amino acid that corresponds to V29 of SEQ ID NO: 2 is C, and the amino acid that corresponds to K110 of SEQ ID NO: 2 is C; (d) the amino acid that corresponds to D31 of SEQ ID NO: 2 is C, and the amino acid that corresponds to S62 of SEQ ID NO: 2 is C; (e) the amino acid that corresponds to D31 of SEQ ID NO: 2 is C, and the amino acid that corresponds to A63 of SEQ ID NO: 2 is C; (f) the amino acid that corresponds to V33 of SEQ ID NO: 2 is C, and the amino acid that corresponds to K61 of SEQ ID NO: 2 is C; (g) the amino acid that corresponds to V33 of SEQ ID NO: 2 is C, and the amino acid that corresponds to A63 of SEQ ID NO: 2 is C; (h) the amino acid that corresponds to F36 of SEQ ID NO: 2 is C, and the amino acid that corresponds to K106 of SEQ ID NO: 2 is C; (i) the amino acid that corresponds to F36 of SEQ ID NO: 2 is C, and the amino acid that corresponds to K107 of SEQ ID NO: 2 is C; (j) the amino acid that corresponds to A39 of SEQ ID NO: 2 is C, and the amino acid that corresponds to E105 of SEQ ID NO: 2 is C; (k) the amino acid that corresponds to A39 of SEQ ID NO: 2 is C, and the amino acid that corresponds to K107 of SEQ ID NO: 2 is C; (1) the amino acid that corresponds to A39 of SEQ ID NO: 2 is C, and the amino acid that corresponds to F112 of SEQ ID NO: 2 is C; and (m) the amino acid that corresponds to Q56 of SEQ ID NO: 2 is C, and the amino acid that corresponds to K80 of SEQ ID NO: 2 is C.

In some embodiments, at least 1, at least 2, at least 3, at least 4, at least 5, at least 6, at least 7, at least 8, at least 9, at least 10, at least 11, or all of the amino acids corresponding to positions 12, 16, 19, 23, 105, 114, 118, 121, 122, 124, 125, and 128 of SEQ ID NO: 2 in the engineered IL-21 polypeptide are not mutated. For example, in some embodiments, the amino acid corresponding to position 12 (e.g., M12) of SEQ ID NO: 2 in the engineered IL-21 polypeptide described herein is Met; the amino acid corresponding to position 16 (e.g., R16) of SEQ ID NO: 2 in the engineered IL-21 polypeptide described herein is Arg; the amino acid corresponding to position 19 (e.g., 119) of SEQ ID NO: 2 in the engineered IL-21 polypeptide described herein is Ile; the amino acid corresponding to position 23 (e.g., D23) of SEQ ID NO: 2 in the engineered IL-21 polypeptide described herein is Asp; the amino acid corresponding to position 105 (e.g., E105) of SEQ ID NO: 2 in the engineered IL-21 polypeptide described herein is Glu; the amino acid corresponding to position 114 (e.g., E114) of SEQ ID NO: 2 in the engineered IL-21 polypeptide described herein is Glu; the amino acid corresponding to position 118 (e.g., Q121) of SEQ ID NO: 2 in the engineered IL-21 polypeptide described herein is Gln; the amino acid corresponding to position 122 (e.g., K122) of SEQ ID NO: 2 in the engineered IL-21 polypeptide described herein is Lys; the amino acid corresponding to position 124 (e.g., 1124) of SEQ ID NO: 2 in the engineered IL-21 polypeptide described herein is Ile; the amino acid corresponding to position 125 (e.g., H125) of SEQ ID NO: 2 in the engineered IL-21 polypeptide described herein is His; and/or the amino acid corresponding to position 128 (e.g., L128) of SEQ ID NO: 2 in the engineered IL-21 polypeptide described herein is Leu.

In some embodiments, the engineered IL-21 polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 3, 4, 5, 6, 7, 8, 9, 10, 11, 12, 13, 14, or 15. In some embodiments, the engineered IL-21 polypeptide described herein comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 1 or SEQ ID NO: 2, wherein the amino acid sequence comprises one or more of the mutations described herein. In some embodiments, the engineered IL-21 polypeptide described herein includes at least 1, at least 2, at least 3, at least 4, or at least 5 pairs of the cysteine mutations described in Table 1.

In some embodiments, the engineered IL-21 polypeptide can have at least or about 1 (e.g., at least or about 2, 3, 4, 5, 6, 7, 8, 9, 10, 20, 30, 40) amino acid insertions, deletions, or substitutions as compared to any one of SEQ ID NOs: 2-15.

The engineered IL-21 polypeptide can have additional modifications. In some embodiments, the engineered IL-21 polypeptide can have a CH2 domain and/or a CH3 domain of Fc. In some embodiments, the engineered IL-21 polypeptide can be linked to the N-terminus of the CH2 domain (e.g., through an optional hinge region or a GS linker). In some embodiments, the engineered IL-21 polypeptide can be linked to the C-terminus of the CH3 domain (e.g., through an optional GS linker). In some embodiments, the hinge region is an IgG hinge region (e.g., IgG4 hinge region). In some embodiments, the CH2 domain is an IgG CH2 domain (e.g., IgG4 CH2 domain). In some embodiments, the CH3 domain is an IgG CH3 domain (e.g., IgG4 CH3 domain).

In some embodiments, the engineered IL-21 polypeptides described herein can also include a tag (e.g., His tag) to facilitate screening and/or detection. In some embodiments, the tag has a sequence that is at least 80%, 85%, 90%, 95%, 96%, 97%, 98%, 99%, or 100% identical to any one of SEQ ID NOs: 22. In some embodiments, the tag is connected to the N-terminus or C-terminus of any engineered IL-21 polypeptides described herein.

In some embodiments, the engineered IL-21 polypeptide described herein can be expressed in Expi293 or CHO-S cells.

### IL-21 Protein Constructs

The disclosure provides protein constructs (e.g., fusion proteins or protein complexes) comprising the engineered IL-21 variants described herein (e.g., any of the engineered IL-21 polypeptides described herein). In some embodiments, the protein constructs further include a human serum albumin (HSA) that is fused to the engineered IL-21 polypeptide. In some embodiments, the fusion protein can be expressed in Expi293 or CHO-S cells. In some embodiments, the HSA is fused to the engineered IL-21 polypeptide via a linker peptide (e.g., a flexible linker). In some embodiments, the HSA is linked to the N-terminus of the engineered IL-21 polypeptide via a linker peptide. In some embodiments, the HSA is linked to the C-terminus of the engineered IL-21 polypeptide via a linker peptide. In some embodiments, the protein constructs described herein have a N-terminal His-tag. In some embodiments, the protein constructs described herein have a C-terminal His-tag.

In some embodiments, the protein construct described herein includes, from N-terminus to C-terminus: (a) optionally a His-tag; (b) a HSA; (c) a linker peptide; and (d) an engineered IL-21 polypeptide (e.g., any of the engineered IL-21 polypeptides described herein). In some embodiments, the His-tag includes at least 6, at least 7, or at least 8 contiguous His residues. In some embodiments, the His-tag comprises or consists of an amino acid sequence that is at least 85%, 90%, 95%, 96%, 97%, 98%, 99%, or 100% to SEQ ID NO: 22. In some embodiments, the HSA is a wild-type HSA or a fragment thereof. In some embodiments, the HSA comprises or consists of an amino acid sequence that is at least 85%, 90%, 95%, 96%, 97%, 98%, 99%, or 100% to SEQ ID NO: 26. In some embodiments, the linker peptide comprises at least 1, 2, 3, 4, 5, 6, 7, or 8 repeats of GGGGS (SEQ ID NO: 27). In some embodiments, the linker peptide is a flexible linker. Details of flexible linkers can be found, e.g., Chen, X., et al. “Fusion protein linkers: property, design and functionality.” Advanced Drug Delivery Reviews 65.10 (2013): 1357-1369, which is incorporated herein by reference in its entirety. In some embodiments, the linker peptide comprises or consists of an amino acid sequence that is at least 85%, 90%, 95%, 96%, 97%, 98%, 99%, or 100% identical to SEQ ID NO: 25. In some embodiments, the engineered IL-21 polypeptide comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 2, 3, 4, 5, 6, 7, 8, 9, 10, 11, 12, 13, 14, or 15. In some embodiments, the engineered IL-21 polypeptide described herein includes at least 1, at least 2, at least 3, at least 4, or at least 5 pairs of the cysteine mutations described in Table 1.

In some embodiments, the engineered IL-21 protein constructs can comprise any engineered IL-21 variant as described herein. In some embodiments, the protein construct described herein comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 17, 18, 19, 20, or 21.

In some embodiments, provided herein are protein constructs that include, from N-terminus to C-terminus, a His-tag (e.g., any of the His-tags described herein), and an engineered IL-21 polypeptide (e.g., any of the engineered IL-21 polypeptides described herein). In some embodiments, the protein construct described herein comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 24.

The disclosure also provides a nucleic acid comprising a polynucleotide encoding a polypeptide comprising a sequence that is at least 80%, 81%, 82%, 83%, 84%, 85%, 86%, 87%, 88%, 89% 90%, 91%, 92%, 93%, 94%, 95%, 96%, 97%, 98%, 99% or 100% identical to any sequence of SEQ ID NOs: 2-15; or any sequence of SEQ ID NOs: 17-21.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The engineered IL-21 variants (e.g., any of the engineered IL-21 polypeptides described herein) and protein constructs can further comprises an Fc region of an antibody. These antibodies can be of any type (e.g., IgG, IgE, IgM, IgD, IgA, and IgY), class or subclass (e.g., IgG1, IgG2, IgG3, IgG4, IgA1, IgA2, IgE1, IgE2). In some embodiments, the Fc region is derived from human IgG (e.g., IgG1, IgG2, IgG3, or IgG4). In some embodiments, the Fc region is an IgG4 Fc region (e.g., human IgG4 Fc region).

In some embodiments, the engineered IL-21 variant is linked to the Fc region through an antibody hinge region (e.g., IgG, IgE hinge region). In addition, the Fc region can be modified to provide desired effector functions or serum half-life.

In some embodiments, the protein constructs as described herein include a functional Fc region. In some embodiments, the Fc region is human IgG1, human IgG2, human IgG3, or human IgG4. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis. In some embodiments, the protein constructs as described herein have an Fc region without effector function. In some embodiments, the Fc is a human IgG4 Fc. In some embodiments, the Fc does not have a functional Fc region. For example, the Fc region has LALA mutations (L234A and L235A mutations in EU numbering), or LALA-PG mutations (L234A, L235A, P329G mutations in EU numbering).

In some embodiments, the engineered IL-21 variant (e.g., any of the engineered IL-21 variants described herein) is linked to the N-terminus or C-terminus of the Fc region. In some embodiments, the engineered IL-21 variant is linked to the Fc region via a linker peptide (e.g., any of the linker peptides described herein).

In some embodiments, provided herein are protein constructs that include, from N-terminus to C-terminus, a human IgG4 hinge region and Fc region (e.g., SEQ ID NO: 28), a linker peptide (e.g., any of the linker peptides described herein), and an engineered IL-21 polypeptide (e.g., any of the engineered IL-21 polypeptides described herein). In some embodiments, the engineered IL-21 polypeptide comprises or consists of a sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to any one of SEQ ID NOs: 2-15. In some embodiments, the protein construct described herein comprises or consists of an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 16.

Some other modifications to the Fc region can be made. For example, a cysteine residue(s) can be introduced into the Fc region, thereby allowing interchain disulfide bond formation in this region. The homodimeric fusion protein thus generated may have any increased half-life in vitro and/or in vivo. In some embodiments, the IgG4 has S228P mutation (EU numbering). The S228P mutation prevents in vivo and in vitro IgG4 Fab-arm exchange.

In some embodiments, Fc regions are provided having a carbohydrate structure that lacks fucose attached (directly or indirectly) to an Fc region. For example, the amount of fucose in such Fc region composition may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues; or position 314 in Kabat numbering); however, Asn297 may also be located about +3 amino acids upstream or downstream of position 297, i.e., between positions 294 and 300, due to minor sequence variations in Fc region sequences. Such fucosylation variants may have improved ADCC function. In some embodiments, to reduce glycan heterogeneity, the Fc region can be further engineered to replace the Asparagine at position 297 with Alanine (N297A).

In some embodiments, the disclosure is related to a protein construct comprising the engineered IL-21 polypeptide described herein. In some embodiments, the protein construct comprises two or more engineered IL-21 polypeptides. In some embodiments, at least two of the engineered IL-21 polypeptides are identical. In some embodiments, at least two of the engineered IL-21 polypeptides are different. In some embodiments, the protein construct further comprises an Fc region. In some embodiments, the Fc region is an IgG4 Fc region. In some embodiments, the Fc region is an IgG1 Fc region (e.g., with LALA mutations or LALA-PG mutations). In some embodiments, the engineered IL-21 polypeptide is linked to the C-terminus of the Fc region. In some embodiments, the engineered IL-21 polypeptide is linked to the C-terminus of the Fc region via a linker peptide (e.g., any of the linker peptides described herein).

The disclosure provides protein complexes comprising the engineered IL-21 variants described herein (e.g., any of the engineered IL-21 polypeptides described herein). In some embodiments, the disclosure is related to a protein complex comprising a first polypeptide and a second polypeptide. In some embodiments, the first polypeptide comprises or consists of, optionally from N-terminus to C-terminus: an optional first hinge region (e.g., a human IgG4 hinge region), a first Fc region (e.g., a human IgG4 Fc region), an optional linker peptide (e.g., any of the linker peptides described herein), and an engineered IL-21 polypeptide (e.g., any of the engineered IL-21 polypeptides described herein). In some embodiments, the second polypeptide comprises or consists of, optionally from N-terminus to C-terminus: an optional second hinge region (e.g., a human IgG4 hinge region), and a second Fc region (e.g., a human IgG4 Fc region). In some embodiments, the first Fc region and/or the second Fc region described herein do not include a hinge region. In some embodiments, the first Fc region and/or the second Fc region described herein consists of a CH2 domain (e.g., a human IgG4 CH2 domain) and a CH3 domain (e.g., a human IgG4 CH3 domain).

In some embodiments, the first and/or the second hinge region include all or a portion of the hinge region of an immunoglobulin, e.g., human IgG4 hinge region (SEQ ID NO: 31). In some embodiments, the first and/or the second hinge region include an amino acid sequence that is at least 80%, 85%, 90%, 91%, 92%, 93%, 94%, 95%, 96%, 97%, 98%, 99%, or 100% identical to SEQ ID NO: 31. In some embodiments, the first and the second hinge regions are identical. In some embodiments, the first and the second hinge regions are different.

In some embodiments, the first and/or the second Fc region are identical and can form a Fc homodimer. In some embodiments, the first and/or the second Fc region include all or a portion of the Fc region of an immunoglobulin, e.g., human IgG4 Fc region (SEQ ID NO: 40). In some embodiments, the first and/or the second Fc region are different. In some embodiments, the first and/or the second Fc region can form a Fc heterodimer by introducing one or more mutations. In some cases, the first and/or the second Fc region can include one or more knob-into-hole (KIH) mutations. For example, the first Fc region can include a cysteine at position 349, a serine at position 366, an alanine at position 368, and a valine at position 407 according to EU numbering; and the second Fc region can include a cysteine at position 354 and a tryptophan at position 366 according to EU numbering. In some embodiments, the first Fc region includes an amino acid sequence that is at least 80%, 85%, 90%, 91%, 92%, 93%, 94%, 95%, 96%, 97%, 98%, 99%, or 100% identical to SEQ ID NO: 33, and the second Fc region includes an amino acid sequence that is at least 80%, 85%, 90%, 91%, 92%, 93%, 94%, 95%, 96%, 97%, 98%, 99%, or 100% identical to SEQ ID NO: 32. In some embodiments, the first and/or the second Fc region can form a Fc heterodimer using other technologies. Details of the KIH mutations and other heterodimeric Fc technologies can be found, e.g., in Ha, et al. “Immunoglobulin Fc heterodimer platform technology: from design to applications in therapeutic antibodies and proteins.” Frontiers In Immunology 7 (2016): 394, which is incorporated herein by reference in its entirety. In some embodiments, the first and/or the second Fc regions described herein are derived from human IgG (e.g., IgG1, IgG2, IgG3, or IgG4). In some embodiments, the first and/or second Fc regions are IgG4 Fc regions (e.g., human IgG4 Fc regions). In some embodiments, the first and/or second Fc regions are IgG Fc regions (e.g., human IgG1 Fc regions) whose effector function is silenced. Details of methods to modulate Fc effector functions can be found, e.g., in Liu, R., et al. “Fc-engineering for modulated effector functions-improving antibodies for cancer treatment.” Antibodies. 2020; 9:64; and Saunders, K. O. “Conceptual approaches to modulating antibody effector functions and circulation half-life. Front Immunol 10:1296.” (2019); each of which is incorporated herein by reference in its entirety.

In some embodiments, the linker peptide described herein includes an amino acid sequence that is at least 80%, 85%, 90%, 95%, or 100% identical to SEQ ID NO: 34. In some embodiments, the linker peptide described herein includes an amino acid sequence that is at least 80%, 85%, 90%, 95%, or 100% identical to one or more (e.g., 1, 2, 3, 4, 5, 6, 7, or 8) repeats of GGGGS (SEQ ID NO: 41).

In some embodiments, the first polypeptide described herein includes an amino acid sequence that is at least 80%, 85%, 90%, 95%, or 100% identical to SEQ ID NO: 30, 35, 36, 37, 38, or 39; and the second polypeptide described herein includes an amino acid sequence that is at least 80%, 85%, 90%, 95%, or 100% identical to SEQ ID NO: 29.

### Characterization of the Engineered IL-21 Variants or Protein Constructs

In some embodiments, the engineered IL-21 variants (e.g., any of the engineered IL-21 polypeptides described herein) or protein constructs (e.g., fusion proteins or protein complexes) thereof described herein can bind to a complex formed by human IL-21 receptor (IL-21R) and common cytokine γ chain (yc). Because the newly introduced non-native disulfide bond can stabilize IL-21 (e.g., the core structure formed by the four-helical bundle) without causing substantial conformational changes of the protein structure, and the residues critical for IL-21R or γc binding (e.g., those corresponding to M12, R16, 119, D23, E105, E114, S118, Q121, K122, 1124, H125, and/or L128 in SEQ ID NO: 2) are unmutated, the engineered IL-21 variants or protein constructs thereof can induce downstream signaling pathways, e.g., the JAK-STAT, MAPK, and/or PI3K pathways, by binding to the IL-21R/yc complex expressed on immune cells (e.g., T cells, B cells, and/or NK cells).

In some embodiments, introduction of the non-native disulfide bond, or cysteine mutations (e.g., any of the cysteine mutations described herein) can lead to protein conformational change with a RMSD (root-mean-square deviation of atomic positions) value of less than 10 Å, less than 9 Å, less than 8 Å, less than 7 Å, less than 6 Å, less than 5 Å, less than 4 Å, less than 3 Å, less than 2 Å, or less than 1 Å. In some embodiments, the RMSD value is calculated by structurally align the wild-type protein and the protein variants. In some embodiments, only Cₐₗₚₕₐ atoms are used for determining the conformational change.

The biological effects of the engineered IL-21 variants or protein constructs thereof include immune enhancing effects and immune regulatory effect. Exemplary immune enhancing effects include, e.g., increasing immune response; increasing B cell proliferation; inducing plasma cell differentiation; increasing immunoglobulin production; increasing T follicular helper (T_{FH}) cell differentiation and/or proliferation; increasing proliferation, survival, and/or anti-tumor activity of cytotoxic T lymphocytes (CTL); increasing CD28 and L-selectin expression; increasing proliferation, anti-tumor activity, and/or ADCC activity of NK cells; increasing differentiation, proliferation, and/or IL-23R expression by Th17 cells; inhibiting survival and/or generation of Treg cells. Exemplary immune regulatory effects include, e.g., suppressing differentiation, proliferation, and/or IL-10 production by type 1 regulatory T (Tr1) cells; suppressing apoptosis, and/or inhibiting APC function of dendritic cells (DC cells); suppressing differentiation, proliferation, and/or IL-10 production by B-10 or B-Regulatory (Breg) cells. More details can be found, e.g., in Croce, M. et al. “IL-21: a pleiotropic cytokine with potential applications in oncology.” Journal of Immunology Research 2015 (2015), which is incorporated herein by reference in its entirety.

In some embodiments, the engineered IL-21 variants (e.g., any of the engineered IL-21 polypeptides described herein) or protein constructs thereof described herein (e.g., any of the HSA-fused IL-21 variants described herein or heterodimeric Fc-fused IL-21 variants described herein) have a comparable potency to induce T cell response. In some embodiments, the engineered IL-21 variants or protein constructs thereof can induce STAT3 phosphorylation of immune cells (e.g., activated CD3+ or CD8+ T cells) with a potency that is at least about 60%, about 70%, about 80%, about 90%, about 100%, about 110%, about 120%, about 130%, or about 140% as compared to that of a wild-type IL-21 or protein construct thereof. In some embodiments, the EC50 value of the proliferation curves can be determined, e.g., less than 0.1 nM, less than 0.09 nM, less than 0.08 nM, less than 0.07 nM, or less than 0.06 nM.

In some embodiments, the engineered IL-21 variants or protein constructs thereof described herein (e.g., any of the HSA-fused IL-21 variants described herein or heterodimeric Fc-fused IL-21 variants described herein) can induce proliferation of immune cells (e.g., activated CD3+ or CD8+ T cells) with a potency that is at least about 60%, about 70%, about 80%, about 90%, about 100%, about 110%, about 120%, about 130%, about 140%, about 150%, about 160%, about 170%, about 180%, about 190%, or about 200% as compared to that of a wild-type IL-21 or protein construct thereof. In some embodiments, the EC50 value of the proliferation curves can be determined, e.g., less than 1 nM, less than 0.5 nM, less than 0.4 nM, or less than 0.3 nM. In some embodiments, the EC50 value of the proliferation curve of activated CD8+ T cells treated with the engineered IL-21 variants or protein constructs thereof described herein is less than 50%, less than 40%, less than 30%, or less than 20% of that of activated CD8+ T cells treated with a wild-type IL-21 or protein construct thereof.

In some embodiments, the engineered IL-21 variants described herein includes an amino acid sequence that is at least 80%, 85%, 90%, 95%, or 100% identical to SEQ ID NO: 3, 5, 9, 10, or 13. In some embodiments, the protein constructs (e.g., HSA-fused IL-21 variants) described herein includes an amino acid sequence that is at least 80%, 85%, 90%, 95%, or 100% identical to SEQ ID NO: 17, 18, 19, 20, or 21. In some embodiments, the protein constructs (e.g., heterodimeric Fc-fused IL-21 variants) includes a first polypeptide having an amino acid sequence that is at least 80%, 85%, 90%, 95%, or 100% identical to SEQ ID NO: 35, 36, 37, 38, or 39; and second polypeptide having an amino acid sequence that is at least 80%, 85%, 90%, 95%, or 100% identical to SEQ ID NO: 29.

In some embodiments, the engineered IL-21 variants or protein constructs thereof described herein (e.g., any of the HSA-fused IL-21 variants described herein or heterodimeric Fc-fused IL-21 variants described herein) can induce proliferation of NK cells (e.g., NK-92 cells) with a potency that is about 60%, about 70%, about 80%, about 90%, about 100%, about 110%, about 120%, about 130%, about 140%, about 150%, about 160%, about 170%, about 180%, about 190%, or about 200% as compared to that of a wild-type IL-21 or protein construct thereof. In some embodiments, the proliferation of NK cells is determined after starvation for at least 12 hours, 18 hours, 24 hours, 30 hours, or 36 hours. In some embodiments, the EC50 value of the proliferation curves can be determined, e.g., less than 0.2 nM, less than 0.15 nM, less than 0.14 nM, less than 0.13 nM, less than 0.12 nM, less than 0.11 nM, less than 0.1 nM, less than 0.09 nM, less than 0.08 nM, less than 0.07 nM, less than 0.06 nM, or less than 0.05 nM. In some embodiments, the EC50 value of the proliferation curves can be determined, e.g., less than 8 nM, less than 7 nM, less than 6 nM, or less than 5 nM. In some embodiments, EC50 value of the proliferation curve of NK cells treated with the engineered IL-21 variants or protein constructs thereof described herein is less than 90%, less than 80%, less than 70%, or less than 60% of that of NK cells treated with a wild-type IL-21 or protein construct thereof.

In some embodiments, the engineered IL-21 variants or protein constructs thereof described herein (e.g., any of the HSA-fused IL-21 variants described herein or heterodimeric Fc-fused IL-21 variants described herein) can relieve Treg-mediated suppression of T cell (e.g., activated CD8+ T cells). In some embodiments, upon treatment with the engineered IL-21 variants or protein constructs thereof described herein and in the presence of Treg cells, the proliferation of T cells (e.g., activated CD8+ T cells) can be enhanced by at least 5%, at least 10%, at least 15%, at least 20%, at least 25%, at least 30%, at least 35%, at least 40%, at least 45%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 250%, or at least 300% as compared to a wild-type IL-21 or protein construct thereof.

In some embodiments, the engineered IL-21 variants or protein constructs thereof described herein (e.g., any of the HSA-fused IL-21 variants described herein or heterodimeric Fc-fused IL-21 variants described herein) can induce cytotoxicity of NK cells (e.g., primary NK cells) with a potency that is about 60%, about 70%, about 80%, about 90%, about 100%, about 110%, about 120%, about 130%, or about 140% as compared to that of a wild-type IL-21 or protein construct thereof. In some embodiments, the specific lysis percentage of the NK cells against target tumor cells (e.g., K-562 tumor cells) can be enhanced by at least at least 5%, at least 10%, at least 15%, at least 20%, at least 25%, at least 30%, at least 35%, at least 40%, at least 45%, at least 50%, at least 60%, at least 70%, at least 80%, at least 90%, or at least 100% as compared to a wild-type IL-21 or protein construct thereof.

In some embodiments, the engineered IL-21 variants or protein constructs thereof as described herein can increase immune response, activity or number of immune cells (e.g., T cells or NK cells) by at least 10%, 20%, 30%, 40%, 50%, 60%, 70%, 80%, 90%, 100%, 2 folds, 3 folds, 5 folds, 10 folds, or 20 folds.

In some implementations, the engineered IL-21 variants or protein constructs thereof can bind to IL-21R, γc, or the complex thereof (e.g., human IL-21R/γc complex) with a dissociation rate (k_{off}) of less than 0.1 s⁻¹, less than 0.01 s⁻¹, less than 0.001 s⁻¹, less than 0.0001 s⁻¹, or less than 0.00001 s⁻¹. In some embodiments, the dissociation rate (k_{off}) is greater than 0.01 s⁻¹, greater than 0.001 s⁻¹, greater than 0.0001 s⁻¹, greater than 0.00001 s⁻¹, or greater than 0.000001 s⁻¹.

In some embodiments, kinetic association rates (kₒₙ) is greater than 1×10²/Ms, greater than 1×10³/Ms, greater than 1×10⁴/Ms, greater than 1×10⁵/Ms, or greater than 1×106/Ms. In some embodiments, kinetic association rates (kₒₙ) is less than 1×10⁵/Ms, less than 1×10⁶/Ms, or less than 1×10⁷/Ms.

Affinities can be deduced from the quotient of the kinetic rate constants (KD=k_{off}/kₒₙ). In some embodiments, KD is less than 1×10⁻⁶ M, less than 1× 10-7M, less than 1× 10-8 M, less than 1×10⁻⁹ M, or less than 1×10⁻¹⁰ M. In some embodiments, the KD is less than 300 nM, 200 nM, 100 nM, 50 nM, 30 nM, 20 nM, 15 nM, 10 nM, 9 nM, 8 nM, 7 nM, 6 nM, 5 nM, 4 nM, 3 nM, 2 nM, 1 nM, 900 pM, 800 pM, 700 pM, 600 pM, 500 pM, 400 pM, 300 pM, 200 pM, 100 pM, 90 pM, 80 pM, 70 pM, 60 pM, 50 pM, 40 pM, 30 pM, 20 pM, or 10 pM. In some embodiments, KD is greater than 1×10⁻⁷M, greater than 1×10⁻⁸ M, greater than 1×10⁻⁹ M, greater than 1×10⁻¹⁰ M, greater than 1× 10⁻¹¹ M, or greater than 1× 10⁻¹² M.

General techniques for measuring the affinity include, e.g., ELISA, radioimmunoassay (RIA), and surface plasmon resonance (SPR). In some embodiments, the affinity is determined by cell-based assays.

In some embodiments, the engineered IL-21 protein constructs thereof described herein (e.g., any of the HSA-fused IL-21 variants described herein) can be expressed and purified by methods commonly used in the art, e.g., affinity chromatography. In some cases, the protein constructs can be purified by size-exclusive chromatography (SEC) coupled with HPLC. In some embodiments, the percentage of the main peak in the SEC-HPLC analysis result is at least 80%, at least 90%, at least 95%, at least 96%, at least 96%, at least 97%, at least 98%, or at least 99%. In some embodiments, the percentage of high molecular weight peak (HMW %) and/or low molecular weight peak (LMW %) is less than 5%, less than 4%, less than 3%, less than 2%, or less than 1%.

In some embodiments, thermal stabilities of the engineered IL-21 variants or protein constructs thereof described herein (e.g., any of the HSA-fused IL-21 variants described herein) are determined. The engineered IL-21 variants and protein constructs thereof described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, the aggregation (T_{agg}) and onset (Tₒₙₛₑₜ) temperatures of the engineered IL-21 variants and protein constructs thereof described herein can be measured by application on a heat ramp (e.g., from 25-85° C.) based on DLS/SLS. DLS/SLS are well-known techniques for determining sample interactions, particle size, and aggregation of molecules dispersed or dissolved in solution. The temperatures that experience the onset of unfolding (Tₒₙₛₑₜ) and aggregation (T_{agg}) are considered key predictors of stability. In some embodiments, the T_{agg} of the engineered IL-21 variants or protein constructs thereof described herein is at least 56, 57, 58, 59, 60, 61, 62, 63, 64, or 65° C. In some embodiments, the Tₒₙₛₑₜ of the engineered IL-21 variants or protein constructs thereof described herein is at least 59, 60, 61, 62, 63, 64, 65, or 66° C.

In some embodiments, the cysteine mutations (e.g., any of the cysteine mutations or a combination thereof described herein) can increase the aggregation (T_{agg}) and/or onset (Tₒₙₛₑₜ) temperatures by at least 0.5° C., at least 1° C., at least 1.5° C., at least 2° C., at least 2.5° C., at least 3° C., at least 3.5° C., at least 4° C., at least 4.5° C., or at least 5° C.

The radius of the engineered IL-21 variants or protein constructs thereof described herein (e.g., any of the HSA-fused IL-21 variants described herein) can also be determined by DSL/SLS. In some embodiments, under isothermal conditions (e.g., at about 25° C.), the radius is measured to be about 1-10 nm, about 3-8 nm, about 4-7 nm, about 4-6 nm, about 4-5 nm, about 4.5-7 nm, about 4.5-6 nm, about 4.5-5 nm, about 5-7 nm, about 5-6 nm, or about 6-7 nm. In some embodiments, under thermo ramp conditions (e.g., at a heat ramp from 25-85° C.), the radius is measured to be about 1-20 nm, about 3-15 nm, about 5-13 nm, about 5-10 nm, about 5-8 nm, about 5-7 nm, about 5-6 nm, about 6-13 nm, about 6-10 nm, about 6-7 nm, about 7-13 nm, about 10-13 nm, or about 12-13 nm.

The polydispersity (% PD) of the engineered IL-21 variants or protein constructs thereof described herein (e.g., any of the HSA-fused IL-21 variants described herein) can also be determined by DSL/SLS. In some embodiments, the % PD is less than about 30%, about 28%, about 25%, about 20%, about 15%, about 13%, about 10%, about 8%, or about 5%, under isothermal conditions (e.g., at about 25° C.).

The molecular weight (MW-S) of the engineered IL-21 variants or protein constructs thereof described herein (e.g., any of the HSA-fused IL-21 variants described herein) can also be determined by DLS/SLS. In some embodiments, under isothermal conditions (e.g., at about 25° C.) or thermo ramp conditions (e.g., at a heat ramp from 25-85° C.), the MW-S is about 50-200 kDa, about 50-130 kDA, about 50-100 kDa, about 50-90 kDa, about 50-80 kDa, about 50-70 kDa, about 60-100 kDa, about 60-90 kDa, about 60-80 kDa, about 70-100 kDa, about 70-90 kDa, about 70-80 kDa, about 80-100 kDa, about 90-100 kDa, about 100-200 kDa, about 100-150 kDa, or about 150-200 kDa.

In some embodiments, the engineered IL-21 variants or protein constructs thereof described herein (e.g., any of the HSA-fused IL-21 variants described herein or heterodimeric Fc-fused IL-21 variants described herein) can inhibit tumor growth, e.g., when administered in a tumor-bearing animal. In some cases, the engineered IL-21 variants or protein constructs thereof has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the engineered IL-21 variants or protein constructs thereof described herein has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts. As used herein, the tumor growth inhibition percentage (TG1%) is calculated using the following formula:

TGI(%)=[1−Mean(T_{final}-Tᵢₙᵢₜᵢₐₗ)/Mean(C_{final}-Cᵢₙᵢₜᵢₐₗ)]×100

T_{final} is the average tumor volume in the treatment group on the final day. Tᵢₙᵢₜᵢₐₗ is the average tumor volume in the treatment group on Day 0. C_{final} is the average tumor volume in the control group on the final day. Cᵢₙᵢₜᵢₐₗ is the average tumor volume in the control group on Day 0.

In some embodiments, the TGI % of the engineered IL-21 variants or protein constructs thereof described herein is at least 50%, at least 60%, at least 70%, at least 80%, at least 90%, at least 100%, at least 110%, at least 120%, at least 130%, at least 140%, at least 150%, at least 2-fold, at least 3-fold, at least 4-fold, at least 5-fold, or at least 10-fold greater that of a wild-type IL-21 or protein construct thereof.

In some embodiments, the percentage of tumor-bearing mice treated with the engineered IL-21 variants or protein constructs thereof described herein (e.g., any of the HSA-fused IL-21 variants described herein or heterodimeric Fc-fused IL-21 variants described herein) having a tumor volume that is less than half the value of the mean tumor volume of un-treated tumor-bearing mice is at least 20%, at least 30%, at least 40%, at least 50%, at least 60%, at least 70%, at least 80%, or at least 90% after 14, 15, 16, 17, 18, 19, 20, 21, 22, 23, 24, 25, 26, 27, 28, 29, 30, 31, 32, 33, 34, 35, 36, 37, 38, 39, or 40 days of tumor inoculation, relative to the total number of inoculated mice on Day 0. In some embodiments, the above described percentage is at least 50%, at least 60%, at least 70%, at least 80%, or at least 90% relative to the total number of lived mice on the same days.

In some embodiments, the survival rate of tumor-bearing mice after treatment of the engineered IL-21 variants or protein constructs thereof described herein (e.g., any of the HSA-fused IL-21 variants described herein or heterodimeric Fc-fused IL-21 variants described herein) is at least 30%, at least 40%, at least 50%, at least 60%, at least 70%, at least 80%, or at least 90% after 14, 15, 16, 17, 18, 19, 20, 21, 22, 23, 24, 25, 26, 27, 28, 29, 30, 31, 32, 33, 34, 35, 36, 37, 38, 39, or 40 days of tumor inoculation.

In some embodiments, the engineered IL-21 variants or protein constructs thereof described herein (e.g., any of the HSA-fused IL-21 variants described herein or heterodimeric Fc-fused IL-21 variants described herein) can induce robust tumor-specific memory responses. In some embodiments, the engineered IL-21 variants or protein constructs thereof described herein can inhibit tumor growth after tumor rechallenge (e.g., re-inoculation of the same or different tumor cells) after at least 40, 41, 42, 43, 44, 45, 46, 47, 48, 49, 50, 51, 52, 53, 54, 55, 56, 57, 58, 59, 60, 61, 62, 63, 64 or 65 days.

In some embodiments, the body weight of tumor-bearing mice after treatment of the engineered IL-21 variants or protein constructs thereof described herein (e.g., any of the HSA-fused IL-21 variants described herein or heterodimeric Fc-fused IL-21 variants described herein) is at least 70%, at least 80%, or at least 90% as compared to that of tumor-bearing mice after treatment of a wild-type IL-21 or protein construct thereof.

In some embodiments, the half-life of the engineered IL-21 variants or protein constructs thereof described herein can be increased by at least 10%, at least 20%, at least 30%, at least 40%, at least 50%, at least 60%, at least 70%, at least 80%, at least 90%, at least 1-fold, at least 2-fold, at least 3-fold, at least 4-fold, at least 5-fold, at least 10-fold, at least 20-fold, or at least 100-fold, as compared to that of a wild-type IL-21 or protein construct thereof. In some embodiments, the half-life is determined by measuring the in vivo concentration of the molecule over time, after administration into a subject.

### Methods of Making Engineered IL-21 Variants and Protein Constructs

The engineered IL-21 variants or protein constructs thereof described herein can be prepared by introducing appropriate nucleotide changes into the DNA encoding a IL-21 peptide or a part thereof or by peptide synthesis. Such variants include, for example, deletions, insertions, or substitutions of residues within the amino acids sequences. In some embodiments, selective cysteine mutations can be introduced to one or more pairs of residues whose Cₐₗₚₕₐ atoms are within 3-7 Å angstroms of a 3D structure of human IL-21. In some embodiments, the 3D structure of human IL-21 has a PDB (protein data bank) ID of 3TGX.

Screening can be performed. In a population of such variants, some engineered IL-21 variants can be expressed and purified using methods known in the art. In addition, the potency of some engineered IL-21 variants on T cell response can be determined by STAT-3 phosphorylation and/or T cell proliferation. Based on the experimental results above, some engineered IL-21 variants with a good expression/purification profile and comparable T cell response potency can be selected to generate HSA-fused IL-21 variants (e.g., any of the HSA-fused IL-21 variants described herein).

Screening of the HSA-fused IL-21 variants can be performed. For example, their expression and purification profiles can be compared by various analytical methods (e.g., SDS-PAGE, HPLC-SEC, and/or DSL/SLS). The results can indicate whether the non-native disulfide bond formed by the cysteine mutations in the engineered IL-21 variants, and/or the HSA fusion, can stabilize the HSA fusion protein. For example, some HSA-fused IL-21 variants may have an increased aggregation (T_{agg}) and onset (Tₒₙₛₑₜ) temperatures as compared to HSA-fused wild-type IL-21.

In addition, the potency of the protein constructs described herein (e.g., any of the HSA-fused IL-21 variants or heterodimeric Fc-fused IL-21 variants described herein) can be determined, e.g., by STAT-3 phosphorylation, T cell proliferation, and/or NK cell proliferation. In particular, some HSA-fused IL-21 variants may have a similar or relatively weaker potency on induction of STAT-3 phosphorylation and T cell proliferation than HSA-fused wild-type IL-21. Some HSA-fused IL-21 variants may have a similar or relatively weaker potency on induction of NK cell proliferation than HSA-fused wild-type IL-21. Some heterodimeric Fc-fused IL-21 variants may have a similar potency on induction of STAT-3 phosphorylation than heterodimeric Fc-fused wild-type IL-21. Some heterodimeric Fc-fused IL-21 variants may have a better potency on induction of T cell proliferation and/or NK cell proliferation than heterodimeric Fc-fused wild-type IL-21.

Further, the potency of the protein constructs described herein (e.g., any of the HSA-fused IL-21 variants or heterodimeric Fc-fused IL-21 variants described herein) on relieving Treg-mediated T cell suppression can be determined. In particular, some HSA-fused IL-21 variants may exhibit a significantly enhancement on relieving Treg-mediated T cell suppression than HSA-fused wild-type IL-21. The potency of the HSA-fused IL-21 variants on induction of NK cell cytotoxicity can also be determined. In particular, some HSA-fused IL-21 variants may have a similar or relatively weaker potency on induction of NK cell cytotoxicity than HSA-fused wild-type IL-21. Some heterodimeric Fc-fused IL-21 variants may have a slightly better potency on induction of NK cell cytotoxicity than heterodimeric Fc-fused wild-type IL-21.

Based on the experimental results on HSA-fused IL-21 variants above, some HSA-fused IL-21 variants with a good expression/purification profile, comparable immune cell response potency, enhanced potency to relive Treg-mediated T cell suppression, and/or comparable or reduced potency to induce NK cell cytotoxicity can be selected to evaluate their anti-tumor efficacy and in vivo toxicity, e.g., in a tumor-bearing animal model.

Based on the experimental results on heterodimeric Fc-fused IL-21 variants above, some heterodimeric Fc-fused IL-21 variants with comparable or better immune cell response potency, and/or comparable or better potency to induce NK cell cytotoxicity can be selected to evaluate their anti-tumor efficacy and in vivo toxicity, e.g., in a tumor-bearing animal model.

In some embodiments, the engineered IL-21 variants or protein constructs thereof (e.g., any of the HSA-fused IL-21 variants or heterodimeric Fc-fused IL-21 variants described herein) may have comparable or increased affinity for the IL-21R/γc complex. Any combination of deletions, insertions, and/or combinations can be made to arrive at a variant that has increased binding affinity for the binding partner (e.g., IL-21R). The amino acid changes introduced into the variant can also alter or introduce new post-translational modifications into the polypeptide, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Engineered IL-21 variants can be derived from any species of animal, including mammals. Non-limiting examples of IL-21 variants include IL-21 variants derived from humans, primates, e.g., monkeys and apes, cows, pigs, horses, sheep, camelids (e.g., camels and llamas), chicken, goats, and rodents (e.g., rats, mice, hamsters and rabbits).

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. Techniques for incorporating DNA into such expression systems are well known to those of ordinary skill in the art. The DNA may also be “naked.” The uptake of naked DNA may be increased by coating the DNA onto biodegradable beads that are efficiently transported into the cells.

For expression, the DNA insert comprising a polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. In some embodiments, the promoter is a cytomegalovirus (CMV) promoter. In some embodiments, the promoter is a human promoter, e.g., uHS or HS promoter. The human promoters can improve expression of proteins derived from human. Details of such human promoters can be found, e.g., in Antoniou, M., et al. “Transgenes encompassing dual-promoter CpG islands from the human TBP and HNRPA2B1 loci are resistant to heterochromatin-mediated silencing.” Genomics 82.3 (2003): 269-279; and Zhang, F., et al. “A ubiquitous chromatin opening element (UCOE) confers resistance to DNA methylation-mediated silencing of lentiviral vectors.” Molecular Therapy 18.9 (2010): 1640-1649; each of which is incorporated herein by reference in its entirety. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli, Streptomyces, and Salmonella typhimurium cells; fungal cells, such as yeast cells; insect cells such as Drosophila S2 and Spodoptera Sf9 cells; animal cells such as CHO, COS, Bowes melanoma, and HEK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacl and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH can be used.

Introduction of the construct into the host cell can be affected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

In some embodiments, the host cell is a human cell suitable for protein expression, e.g., HEK293 cells or CHO cells (e.g., CHO-S cells). In some embodiments, the host cells are Expi293 cells. The Expi293 Expression System is designed to deliver up to 6× more protein in just one week, compared with other transient 293 expression systems that can take two weeks or more. This is in part due to the fact that Expi293F cells are adapted to achieve higher pg/cell/day productivity than standard HEK 293 cells, and the Expifectamine 293 Transfection Reagent and enhancers enable high-efficiency transfection and expression levels of high-density HEK 293 cultures. Additionally, the Expi293 Expression System requires less plasticware, which means less waste and more incubator space.

Transcription of DNA encoding a polypeptide of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., engineered IL-21 variants) can be expressed in a modified form, such as a fusion protein (e.g., a HSA-fusion or GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The engineered IL-21 variants and protein constructs thereof of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of engineered IL-21 variants and protein constructs disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer.

In some embodiments, the cancer described herein is esophageal adenocarcinoma, lung cancer, hepatocellular carcinoma, cholangiocarcinoma, gallbladder cancer, gastric cancer, pancreatic cancer, colorectal cancer, endometrial carcinoma, ovarian cancer, bladder cancer, prostate cancer, or T cell lymphoma. In some embodiments, the cancer is renal cancer.

In some embodiments, the compositions and methods disclosed herein can be used for treatment of patients at risk for a cancer. Patients with cancer can be identified with various methods known in the art.

In one aspect, the disclosure provides methods for treating, preventing, or reducing the risk of developing disorders associated with an abnormal or unwanted immune response, e.g., an autoimmune disorder, e.g., by administering a therapeutically effective amount of engineered IL-21 variants and protein constructs disclosed herein to a subject in need thereof. These autoimmune disorders include, but are not limited to, rheumatoid arthritis, Crohn's disease, systemic lupus erythematosus, ankylosing spondylitis, inflammatory bowel diseases (IBD), ulcerative colitis, or scleroderma. In some embodiments, the autoimmune disorders include allergy, asthma, and/or atopic dermatitis. Thus, engineered IL-21 variants and protein constructs disclosed herein can be used to inhibit immune response. In some embodiments, the immune disorders described herein is allergy, asthma, myocarditis, nephritis, hepatitis, systemic lupus erythematosus, rheumatoid arthritis, scleroderma, hyperthyroidism, idiopathic thrombocytopenia purpura, autoimmune hemolytic anemia, ulcerative colitis, autoimmune liver disease, diabetes, pain or neurological disorders.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the engineered IL-21 variants and protein constructs, vector comprising the polynucleotide encoding the engineered IL-21 variants and protein constructs, and/or compositions thereof is to be administered, a severity of symptoms and a route of administration, and thus administration can be determined on an individual basis.

An effective amount can be administered in one or more administrations. By way of example, an effective amount of the engineered IL-21 variants and/or protein constructs is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount may vary, depending on, inter alia, patient history as well as other factors such as the type (and/or dosage) of the engineered IL-21 variants and protein constructs used.

Effective amounts and schedules for administering the engineered IL-21 variants or protein constructs thereof, the polynucleotides encoding the engineered IL-21 variants or protein constructs, and/or compositions disclosed herein may be determined empirically, and making such determinations is within the skill in the art. Those skilled in the art will understand that the dosage that must be administered will vary depending on, for example, the mammal that will receive the engineered IL-21 variants or protein constructs thereof, the polynucleotides, and/or compositions disclosed herein, the route of administration, the particular type of polynucleotides, and/or compositions disclosed herein used and other drugs being administered to the mammal.

A typical daily dosage of an effective amount of the engineered IL-21 variants or protein constructs thereof is 0.1 mg/kg to 200 mg/kg (mg per kg of patient weight). In some embodiments, the dosage can be less than 150 mg/kg, 140 mg/kg, 130 mg/kg, 120 mg/kg, 110 mg/kg, 100 mg/kg, 90 mg/kg, 80 mg/kg, 70 mg/kg, 60 mg/kg, 50 mg/kg, 40 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 150 mg/kg, 140 mg/kg, 130 mg/kg, 120 mg/kg, 110 mg/kg, 100 mg/kg, 90 mg/kg, 80 mg/kg, 70 mg/kg, 60 mg/kg, 50 mg/kg, 40 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage is about 150 mg/kg, 140 mg/kg, 130 mg/kg, 120 mg/kg, 110 mg/kg, 100 mg/kg, 90 mg/kg, 80 mg/kg, 70 mg/kg, 60 mg/kg, 50 mg/kg, 40 mg/kg, 30 mg/kg, 20 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg. In some embodiments, the dosage is about 1 to 150 mg/kg, about 1 to 100 mg/kg, about 1 to 80 mg/kg, about 1 to 50 mg/kg, about 1 to 30 mg/kg, about 1 to 20 mg/kg, about 1 to 10 mg/kg, about 1 to 5 mg/kg, about 5 to 150 mg/kg, about 5 to 100 mg/kg, about 5 to 80 mg/kg, about 5 to 50 mg/kg, about 5 to 30 mg/kg, about 5 to 20 mg/kg, about 5 to 10 mg/kg, about 10 to 150 mg/kg, about 10 to 100 mg/kg, about 10 to 80 mg/kg, about 10 to 50 mg/kg, about 10 to 30 mg/kg, about 10 to 20 mg/kg, about 20 to 150 mg/kg, about 20 to 100 mg/kg, about 20 to 80 mg/kg, about 20 to 50 mg/kg, about 20 to 30 mg/kg, about 30 to 150 mg/kg, about 30 to 100 mg/kg, about 30 to 80 mg/kg, about 30 to 50 mg/kg, about 50 to 150 mg/kg, about 50 to 100 mg/kg, about 50 to 80 mg/kg, about 80 to 150 mg/kg, about 80 to 100 mg/kg, or about 100 to 150 mg/kg.

In any of the methods described herein, the engineered IL-21 variants or protein constructs thereof can be administered to the subject at least once a week (e.g., once a week, twice a week, three times a week, four times a week, once a day, twice a day, or three times a day).

In some embodiments, the one or more additional therapeutic agents can be administered to the subject prior to, or after administering the engineered IL-21 variants or protein constructs thereof. In some embodiments, the one or more additional therapeutic agents are administered to the subject such that there is an overlap in the bioactive period of the one or more additional therapeutic agents and the engineered IL-21 variants or protein constructs thereof in the subject.

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-SIRPa antibody, an anti-CD47 antibody, an anti-LAG3 antibody, an anti-TIGIT antibody, an anti-BTLA antibody, an anti-CTLA-4 antibody, or an anti-GITR antibody. In some embodiments, the additional therapeutic agent is an anti-CD20 antibody (e.g., rituximab) or an anti-EGF receptor antibody (e.g., cetuximab).

### Pharmaceutical Compositions and Routes of Administration

Also provided herein are pharmaceutical compositions that contain the engineered IL-21 variants or protein constructs thereof described herein. The pharmaceutical compositions can be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers. Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the agents can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Compositions containing the engineered IL-21 variants or protein constructs thereof described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, the engineered IL-21 variants or protein constructs thereof can be formulated in aqueous solutions, preferably in physiologically-compatible buffers to reduce discomfort at the site of injection. The solution can contain formulatory agents such as suspending, stabilizing and/or dispersing agents. Alternatively the engineered IL-21 variants or protein constructs thereof can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50: ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Exemplary doses include milligram or microgram amounts of any of the engineered IL-21 variants or protein constructs thereof described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; about 1 μg/kg to about 50 μg/kg; about 1 mg/kg to about 10 mg/kg; or about 1 mg/kg to about 5 mg/kg). While these doses cover a broad range, one of ordinary skill in the art will understand that therapeutic agents can vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the engineered IL-21 variants or protein constructs thereof in vivo.

The pharmaceutical compositions can be included in a container, pack, or dispenser together with instructions for administration. The disclosure also provides methods of manufacturing the engineered IL-21 variants or protein constructs thereof for various uses as described herein.

### Methods of Engineering Cytokines

Provided herein are methods of improving the properties (e.g., stability, activity or affinity) of a protein (e.g., a cytokine), comprising one or more of the following steps: (a) providing a 3D structure of the protein (e.g., the cytokine), (b) measuring distance of the Cₐₗₚₕₐ atoms of one or more of amino acid residues in the 3D structure; and (c) selecting two amino acid residues from the one or more amino acid residues, wherein the Cₐₗₚₕₐ atoms of the two selected amino acid residues are within 3-7 Å. In some embodiments, the Cₐₗₚₕₐ atoms of the two selected amino acid residues are within 4.5-6.5 Å, e.g., about 6.5 Å, 6.4 Å, 6.3 Å, 6.2 Å, 6.1 Å, 6 Å, 5.9 Å, 5.8 Å, 5.7 Å, 5.6 Å, 5.5 Å, 5.4 Å, 5.3 Å, 5.2 Å, 5.1 Å, 5 Å, 4.9 Å, 4.8 Å, 4.7 Å, 4.6 Å, or 4.5 Å. In some embodiments, the directionality from Cₐₗₚₕₐ to C_{beta} atoms of the two selected amino acid residues are in favor of forming a non-native disulfide bond. For example, the C_{beta} atoms point to a direction (from Cₐₗₚₕₐ to C_{beta}) that is possible to form a disulfide bond. In some cases, the angel between the direction of the C_{beta} atom of the first selected amino acid residue (from Cₐₗₚₕₐ to C_{beta} of the first selected amino acid residue) and the direction of the C_{beta} atom of the second selected amino acid residue (from Cₐₗₚₕₐ to C_{beta} of the second selected amino acid residue) is less than 120 degrees. In some cases, the C_{beta} atoms dot not point away from each other. In some embodiments, the 3D structure of the protein (e.g., the cytokine) is derived from a PDB structure of the protein (e.g., the cytokine), a fragment thereof, or a complex of the protein (e.g., the cytokine) bound with its binding partner(s). In some embodiments, the spatial coordinates of all atoms of the 3D structure can be loaded to a software (e.g., a software for modeling and simulation), and the distance of any two atoms in the 3D structure can be determined.

In some embodiments, the angel between the direction of the C_{beta} atom of the first selected amino acid residue and the direction of the C_{beta} atom of the second selected amino acid residue is less than 120 degrees, e.g., less than 120, 119, 118, 117, 116, 115, 114, 113, 112, 111, 110, 109, 108, 107, 106, 105, 104, 103, 102, 101, 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 degree.

In some embodiments, the distance of the Cₐₗₚₕₐ atoms of the two selected amino acid residues described herein is about 3 to about 7 Å, about 3 to about 6.5 Å, about 3 to about 6 Å, about 3 to about 5.5 Å, about 3 to about 5 Å, about 3 to about 4.5 Å, about 3 to about 4 Å, about 3 to about 3.5 Å, about 3.5 to about 7 Å, about 3.5 to about 6.5 Å, about 3.5 to about 6 Å, about 3.5 to about 5.5 Å, about 3.5 to about 5 Å, about 3.5 to about 4.5 Å, about 3.5 to about 4 Å, about 4 to about 7 Å, about 4 to about 6.5 Å, about 4 to about 6 Å, about 4 to about 5.5 Å, about 4 to about 5 Å, about 4 to about 4.5 Å, about 4.5 to about 7 Å, about 4.5 to about 6.5 Å, about 4.5 to about 6 Å, about 4.5 to about 5.5 Å, about 4.5 to about 5 Å, about 5 to about 7 Å, about 5 to about 6.5 Å, about 5 to about 6 Å, about 5 to about 5.5 Å, about 5.5 to about 7 Å, about 5.5 to about 6.5 Å, about 5.5 to about 6 Å, about 6 to about 7 Å, about 6 to about 6.5 Å, or about 6.5 to about 7 Å.

In some embodiments, the distance of the Cₐₗₚₕₐ atoms of the two selected amino acid residues described herein is about 4.5 to about 6.5 Å, about 4.5 to about 6.4 Å, about 4.5 to about 6.3 Å, about 4.5 to about 6.2 Å, about 4.5 to about 6.1 Å, about 4.5 to about 6.0 Å, about 4.5 to about 5.9 Å, about 4.5 to about 5.8 Å, about 4.5 to about 5.7 Å, about 4.5 to about 5.6 Å, about 4.5 to about 5.5 Å, about 4.5 to about 5.4 Å, about 4.5 to about 5.3 Å, about 4.5 to about 5.2 Å, about 4.5 to about 5.1 Å, about 4.5 to about 5.0 Å, about 4.5 to about 4.9 Å, about 4.5 to about 4.8 Å, about 4.5 to about 4.7 Å, about 4.5 to about 4.6 Å, about 4.6 to about 6.5 Å, about 4.6 to about 6.4 Å, about 4.6 to about 6.3 Å, about 4.6 to about 6.2 Å, about 4.6 to about 6.1 Å, about 4.6 to about 6.0 Å, about 4.6 to about 5.9 Å, about 4.6 to about 5.8 Å, about 4.6 to about 5.7 Å, about 4.6 to about 5.6 Å, about 4.6 to about 5.5 Å, about 4.6 to about 5.4 Å, about 4.6 to about 5.3 Å, about 4.6 to about 5.2 Å, about 4.6 to about 5.1 Å, about 4.6 to about 5.0 Å, about 4.6 to about 4.9 Å, about 4.6 to about 4.8 Å, about 4.6 to about 4.7 Å, about 4.7 to about 6.5 Å, about 4.7 to about 6.4 Å, about 4.7 to about 6.3 Å, about 4.7 to about 6.2 Å, about 4.7 to about 6.1 Å, about 4.7 to about 6.0 Å, about 4.7 to about 5.9 Å, about 4.7 to about 5.8 Å, about 4.7 to about 5.7 Å, about 4.7 to about 5.6 Å, about 4.7 to about 5.5 Å, about 4.7 to about 5.4 Å, about 4.7 to about 5.3 Å, about 4.7 to about 5.2 Å, about 4.7 to about 5.1 Å, about 4.7 to about 5.0 Å, about 4.7 to about 4.9 Å, about 4.7 to about 4.8 Å, about 4.8 to about 6.5 Å, about 4.8 to about 6.4 Å, about 4.8 to about 6.3 Å, about 4.8 to about 6.2 Å, about 4.8 to about 6.1 Å, about 4.8 to about 6.0 Å, about 4.8 to about 5.9 Å, about 4.8 to about 5.8 Å, about 4.8 to about 5.7 Å, about 4.8 to about 5.6 Å, about 4.8 to about 5.5 Å, about 4.8 to about 5.4 Å, about 4.8 to about 5.3 Å, about 4.8 to about 5.2 Å, about 4.8 to about 5.1 Å, about 4.8 to about 5.0 Å, about 4.8 to about 4.9 Å, about 4.9 to about 6.5 Å, about 4.9 to about 6.4 Å, about 4.9 to about 6.3 Å, about 4.9 to about 6.2 Å, about 4.9 to about 6.1 Å, about 4.9 to about 6.0 Å, about 4.9 to about 5.9 Å, about 4.9 to about 5.8 Å, about 4.9 to about 5.7 Å, about 4.9 to about 5.6 Å, about 4.9 to about 5.5 Å, about 4.9 to about 5.4 Å, about 4.9 to about 5.3 Å, about 4.9 to about 5.2 Å, about 4.9 to about 5.1 Å, about 4.9 to about 5.0 Å, about 5.0 to about 6.5 Å, about 5.0 to about 6.4 Å, about 5.0 to about 6.3 Å, about 5.0 to about 6.2 Å, about 5.0 to about 6.1 Å, about 5.0 to about 6.0 Å, about 5.0 to about 5.9 Å, about 5.0 to about 5.8 Å, about 5.0 to about 5.7 Å, about 5.0 to about 5.6 Å, about 5.0 to about 5.5 Å, about 5.0 to about 5.4 Å, about 5.0 to about 5.3 Å, about 5.0 to about 5.2 Å, about 5.0 to about 5.1 Å, about 5.1 to about 6.5 Å, about 5.1 to about 6.4 Å, about 5.1 to about 6.3 Å, about 5.1 to about 6.2 Å, about 5.1 to about 6.1 Å, about 5.1 to about 6.0 Å, about 5.1 to about 5.9 Å, about 5.1 to about 5.8 Å, about 5.1 to about 5.7 Å, about 5.1 to about 5.6 Å, about 5.1 to about 5.5 Å, about 5.1 to about 5.4 Å, about 5.1 to about 5.3 Å, about 5.1 to about 5.2 Å, about 5.2 to about 6.5 Å, about 5.2 to about 6.4 Å, about 5.2 to about 6.3 Å, about 5.2 to about 6.2 Å, about 5.2 to about 6.1 Å, about 5.2 to about 6.0 Å, about 5.2 to about 5.9 Å, about 5.2 to about 5.8 Å, about 5.2 to about 5.7 Å, about 5.2 to about 5.6 Å, about 5.2 to about 5.5 Å, about 5.2 to about 5.4 Å, about 5.2 to about 5.3 Å, about 5.3 to about 6.5 Å, about 5.3 to about 6.4 Å, about 5.3 to about 6.3 Å, about 5.3 to about 6.2 Å, about 5.3 to about 6.1 Å, about 5.3 to about 6.0 Å, about 5.3 to about 5.9 Å, about 5.3 to about 5.8 Å, about 5.3 to about 5.7 Å, about 5.3 to about 5.6 Å, about 5.3 to about 5.5 Å, about 5.3 to about 5.4 Å, about 5.4 to about 6.5 Å, about 5.4 to about 6.4 Å, about 5.4 to about 6.3 Å, about 5.4 to about 6.2 Å, about 5.4 to about 6.1 Å, about 5.4 to about 6.0 Å, about 5.4 to about 5.9 Å, about 5.4 to about 5.8 Å, about 5.4 to about 5.7 Å, about 5.4 to about 5.6 Å, about 5.4 to about 5.5 Å, about 5.5 to about 6.5 Å, about 5.5 to about 6.4 Å, about 5.5 to about 6.3 Å, about 5.5 to about 6.2 Å, about 5.5 to about 6.1 Å, about 5.5 to about 6.0 Å, about 5.5 to about 5.9 Å, about 5.5 to about 5.8 Å, about 5.5 to about 5.7 Å, about 5.5 to about 5.6 Å, about 5.6 to about 6.5 Å, about 5.6 to about 6.4 Å, about 5.6 to about 6.3 Å, about 5.6 to about 6.2 Å, about 5.6 to about 6.1 Å, about 5.6 to about 6.0 Å, about 5.6 to about 5.9 Å, about 5.6 to about 5.8 Å, about 5.6 to about 5.7 Å, about 5.7 to about 6.5 Å, about 5.7 to about 6.4 Å, about 5.7 to about 6.3 Å, about 5.7 to about 6.2 Å, about 5.7 to about 6.1 Å, about 5.7 to about 6.0 Å, about 5.7 to about 5.9 Å, about 5.7 to about 5.8 Å, about 5.8 to about 6.5 Å, about 5.8 to about 6.4 Å, about 5.8 to about 6.3 Å, about 5.8 to about 6.2 Å, about 5.8 to about 6.1 Å, about 5.8 to about 6.0 Å, about 5.8 to about 5.9 Å, about 5.9 to about 6.5 Å, about 5.9 to about 6.4 Å, about 5.9 to about 6.3 Å, about 5.9 to about 6.2 Å, about 5.9 to about 6.1 Å, about 5.9 to about 6.0 Å, about 6.0 to about 6.5 Å, about 6.0 to about 6.4 Å, about 6.0 to about 6.3 Å, about 6.0 to about 6.2 Å, about 6.0 to about 6.1 Å, about 6.1 to about 6.5 Å, about 6.1 to about 6.4 Å, about 6.1 to about 6.3 Å, about 6.1 to about 6.2 Å, about 6.2 to about 6.5 Å, about 6.2 to about 6.4 Å, about 6.2 to about 6.3 Å, about 6.3 to about 6.5 Å, about 6.3 to about 6.4 Å, or about 6.4 Å to about 6.5 Å. In some embodiments, the ranges described herein are inclusive, i.e., the values at the boundary of the range are included in the range.

In some embodiments, the distance of the C_{beta} atoms of the two selected amino acid residues described herein is about 3 to about 8 Å, about 3 to about 7.5 Å, about 3 to about 7 Å, about 3 to about 6.5 Å, about 3 to about 6 Å, about 3 to about 5.5 Å, about 3 to about 5 Å, about 3 to about 4.5 Å, about 3 to about 4 Å, about 3 to about 3.5 Å, about 3.5 to about 8 Å, about 3.5 to about 7.5 Å, about 3.5 to about 7 Å, about 3.5 to about 6.5 Å, about 3.5 to about 6 Å, about 3.5 to about 5.5 Å, about 3.5 to about 5 Å, about 3.5 to about 4.5 Å, about 3.5 to about 4 Å, about 4 to about 8 Å, about 4 to about 7.5 Å, about 4 to about 7 Å, about 4 to about 6.5 Å, about 4 to about 6 Å, about 4 to about 5.5 Å, about 4 to about 5 Å, about 4 to about 4.5 Å, about 4.5 to about 8 Å, about 4.5 to about 7.5 Å, about 4.5 to about 7 Å, about 4.5 to about 6.5 Å, about 4.5 to about 6 Å, about 4.5 to about 5.5 Å, about 4.5 to about 5 Å, about 5 to about 8 Å, about 5 to about 7.5 Å, about 5 to about 7 Å, about 5 to about 6.5 Å, about 5 to about 6 Å, about 5 to about 5.5 Å, about 5.5 to about 8 Å, about 5.5 to about 7.5 Å, about 5.5 to about 7 Å, about 5.5 to about 6.5 Å, about 5.5 to about 6 Å, about 6 to about 8 Å, about 6 to about 7.5 Å, about 6 to about 7 Å, about 6 to about 6.5 Å, about 6.5 to about 8 Å, about 6.5 to about 7.5 Å, about 6.5 to about 7 Å, about 7 to about 8 Å, about 7 to about 7.5 Å, or about 7.5 to about 8 Å.

In some embodiments, the distance of the C_{beta} atoms of the two selected amino acid residues described herein is about 4.5 to about 6.5 Å, about 4.5 to about 6.4 Å, about 4.5 to about 6.3 Å, about 4.5 to about 6.2 Å, about 4.5 to about 6.1 Å, about 4.5 to about 6.0 Å, about 4.5 to about 5.9 Å, about 4.5 to about 5.8 Å, about 4.5 to about 5.7 Å, about 4.5 to about 5.6 Å, about 4.5 to about 5.5 Å, about 4.5 to about 5.4 Å, about 4.5 to about 5.3 Å, about 4.5 to about 5.2 Å, about 4.5 to about 5.1 Å, about 4.5 to about 5.0 Å, about 4.5 to about 4.9 Å, about 4.5 to about 4.8 Å, about 4.5 to about 4.7 Å, about 4.5 to about 4.6 Å, about 4.6 to about 6.5 Å, about 4.6 to about 6.4 Å, about 4.6 to about 6.3 Å, about 4.6 to about 6.2 Å, about 4.6 to about 6.1 Å, about 4.6 to about 6.0 Å, about 4.6 to about 5.9 Å, about 4.6 to about 5.8 Å, about 4.6 to about 5.7 Å, about 4.6 to about 5.6 Å, about 4.6 to about 5.5 Å, about 4.6 to about 5.4 Å, about 4.6 to about 5.3 Å, about 4.6 to about 5.2 Å, about 4.6 to about 5.1 Å, about 4.6 to about 5.0 Å, about 4.6 to about 4.9 Å, about 4.6 to about 4.8 Å, about 4.6 to about 4.7 Å, about 4.7 to about 6.5 Å, about 4.7 to about 6.4 Å, about 4.7 to about 6.3 Å, about 4.7 to about 6.2 Å, about 4.7 to about 6.1 Å, about 4.7 to about 6.0 Å, about 4.7 to about 5.9 Å, about 4.7 to about 5.8 Å, about 4.7 to about 5.7 Å, about 4.7 to about 5.6 Å, about 4.7 to about 5.5 Å, about 4.7 to about 5.4 Å, about 4.7 to about 5.3 Å, about 4.7 to about 5.2 Å, about 4.7 to about 5.1 Å, about 4.7 to about 5.0 Å, about 4.7 to about 4.9 Å, about 4.7 to about 4.8 Å, about 4.8 to about 6.5 Å, about 4.8 to about 6.4 Å, about 4.8 to about 6.3 Å, about 4.8 to about 6.2 Å, about 4.8 to about 6.1 Å, about 4.8 to about 6.0 Å, about 4.8 to about 5.9 Å, about 4.8 to about 5.8 Å, about 4.8 to about 5.7 Å, about 4.8 to about 5.6 Å, about 4.8 to about 5.5 Å, about 4.8 to about 5.4 Å, about 4.8 to about 5.3 Å, about 4.8 to about 5.2 Å, about 4.8 to about 5.1 Å, about 4.8 to about 5.0 Å, about 4.8 to about 4.9 Å, about 4.9 to about 6.5 Å, about 4.9 to about 6.4 Å, about 4.9 to about 6.3 Å, about 4.9 to about 6.2 Å, about 4.9 to about 6.1 Å, about 4.9 to about 6.0 Å, about 4.9 to about 5.9 Å, about 4.9 to about 5.8 Å, about 4.9 to about 5.7 Å, about 4.9 to about 5.6 Å, about 4.9 to about 5.5 Å, about 4.9 to about 5.4 Å, about 4.9 to about 5.3 Å, about 4.9 to about 5.2 Å, about 4.9 to about 5.1 Å, about 4.9 to about 5.0 Å, about 5.0 to about 6.5 Å, about 5.0 to about 6.4 Å, about 5.0 to about 6.3 Å, about 5.0 to about 6.2 Å, about 5.0 to about 6.1 Å, about 5.0 to about 6.0 Å, about 5.0 to about 5.9 Å, about 5.0 to about 5.8 Å, about 5.0 to about 5.7 Å, about 5.0 to about 5.6 Å, about 5.0 to about 5.5 Å, about 5.0 to about 5.4 Å, about 5.0 to about 5.3 Å, about 5.0 to about 5.2 Å, about 5.0 to about 5.1 Å, about 5.1 to about 6.5 Å, about 5.1 to about 6.4 Å, about 5.1 to about 6.3 Å, about 5.1 to about 6.2 Å, about 5.1 to about 6.1 Å, about 5.1 to about 6.0 Å, about 5.1 to about 5.9 Å, about 5.1 to about 5.8 Å, about 5.1 to about 5.7 Å, about 5.1 to about 5.6 Å, about 5.1 to about 5.5 Å, about 5.1 to about 5.4 Å, about 5.1 to about 5.3 Å, about 5.1 to about 5.2 Å, about 5.2 to about 6.5 Å, about 5.2 to about 6.4 Å, about 5.2 to about 6.3 Å, about 5.2 to about 6.2 Å, about 5.2 to about 6.1 Å, about 5.2 to about 6.0 Å, about 5.2 to about 5.9 Å, about 5.2 to about 5.8 Å, about 5.2 to about 5.7 Å, about 5.2 to about 5.6 Å, about 5.2 to about 5.5 Å, about 5.2 to about 5.4 Å, about 5.2 to about 5.3 Å, about 5.3 to about 6.5 Å, about 5.3 to about 6.4 Å, about 5.3 to about 6.3 Å, about 5.3 to about 6.2 Å, about 5.3 to about 6.1 Å, about 5.3 to about 6.0 Å, about 5.3 to about 5.9 Å, about 5.3 to about 5.8 Å, about 5.3 to about 5.7 Å, about 5.3 to about 5.6 Å, about 5.3 to about 5.5 Å, about 5.3 to about 5.4 Å, about 5.4 to about 6.5 Å, about 5.4 to about 6.4 Å, about 5.4 to about 6.3 Å, about 5.4 to about 6.2 Å, about 5.4 to about 6.1 Å, about 5.4 to about 6.0 Å, about 5.4 to about 5.9 Å, about 5.4 to about 5.8 Å, about 5.4 to about 5.7 Å, about 5.4 to about 5.6 Å, about 5.4 to about 5.5 Å, about 5.5 to about 6.5 Å, about 5.5 to about 6.4 Å, about 5.5 to about 6.3 Å, about 5.5 to about 6.2 Å, about 5.5 to about 6.1 Å, about 5.5 to about 6.0 Å, about 5.5 to about 5.9 Å, about 5.5 to about 5.8 Å, about 5.5 to about 5.7 Å, about 5.5 to about 5.6 Å, about 5.6 to about 6.5 Å, about 5.6 to about 6.4 Å, about 5.6 to about 6.3 Å, about 5.6 to about 6.2 Å, about 5.6 to about 6.1 Å, about 5.6 to about 6.0 Å, about 5.6 to about 5.9 Å, about 5.6 to about 5.8 Å, about 5.6 to about 5.7 Å, about 5.7 to about 6.5 Å, about 5.7 to about 6.4 Å, about 5.7 to about 6.3 Å, about 5.7 to about 6.2 Å, about 5.7 to about 6.1 Å, about 5.7 to about 6.0 Å, about 5.7 to about 5.9 Å, about 5.7 to about 5.8 Å, about 5.8 to about 6.5 Å, about 5.8 to about 6.4 Å, about 5.8 to about 6.3 Å, about 5.8 to about 6.2 Å, about 5.8 to about 6.1 Å, about 5.8 to about 6.0 Å, about 5.8 to about 5.9 Å, about 5.9 to about 6.5 Å, about 5.9 to about 6.4 Å, about 5.9 to about 6.3 Å, about 5.9 to about 6.2 Å, about 5.9 to about 6.1 Å, about 5.9 to about 6.0 Å, about 6.0 to about 6.5 Å, about 6.0 to about 6.4 Å, about 6.0 to about 6.3 Å, about 6.0 to about 6.2 Å, about 6.0 to about 6.1 Å, about 6.1 to about 6.5 Å, about 6.1 to about 6.4 Å, about 6.1 to about 6.3 Å, about 6.1 to about 6.2 Å, about 6.2 to about 6.5 Å, about 6.2 to about 6.4 Å, about 6.2 to about 6.3 Å, about 6.3 to about 6.5 Å, about 6.3 to about 6.4 Å, or about 6.4 Å to about 6.5 Å. In some embodiments, the ranges described herein are inclusive, i.e., the values at the boundary of the range are included in the range.

In some embodiments, the distance of the Cₐₗₚₕₐ atoms of the two selected amino acid residues are close enough to form a non-native disulfide bond. In some embodiments, the two selected amino acid residues are not involved in the protein (e.g., the cytokine) interaction with its binding partners (e.g., a receptor binding to the protein (e.g., the cytokine)). In some embodiments, the methods further comprise expressing a protein variant (e.g., a cytokine variant), wherein the variant comprises a non-native disulfide bond formed by mutating the two selected amino acid residues to cysteines. In some embodiments, mutating the two selected amino acid residues to cysteines does not substantially change the 3D structure of the protein (e.g., the cytokine). For example, the cysteine mutations do not substantially interfere the overall structure of the protein (e.g., cytokine). In some cases, the mutated residues do not exhibit any spatial clashes with the un-mutated residues. In some cases, a skilled person in the art may perform a simulation of the mutated protein (e.g., the mutated cytokine) structure in silico, and determine the associated conformational change caused by the cysteine mutations. In some embodiments, the associated conformational change is minimal, e.g., with a RMSD value that is considered insignificant by a skilled person in the art.

Also provided herein are methods of screening a cytokine variant with an improved anti-tumor efficacy, comprising (a) providing a 3D structure of the cytokine, (b) measuring distance of the Cₐₗₚₕₐ atoms of one or more of amino acid residues in the 3D structure; and (c) selecting two amino acid residues from the one or more amino acid residues, wherein the Cₐₗₚₕₐ atoms of the two selected amino acid residues are within 7.0 Å, 6.9 Å, 6.8 Å, 6.7 Å, 6.6 Å, 6.5 Å, 6.4 Å, 6.3 Å, 6.2 Å, 6.1 Å, 6 Å, 5.9 Å, 5.8 Å, 5.7 Å, 5.6 Å, 5.5 Å, 5.4 Å, 5.3 Å, 5.2 Å, 5.1 Å, 5 Å, 4.9 Å, 4.8 Å, 4.7 Å, 4.6 Å, or 4.5 Å, 4.4 Å, 4.3 Å, 4.2 Å, 4.1 Å, 4.0 Å, 3.9 Å, 3.8 Å, 3.7 Å, 3.6 Å, 3.5 Å, 3.4 Å, 3.3 Å, 3.2 Å, 3.1 Å, 3.0 Å, or in any of the ranges described herein.

In some embodiments, the protein or cytokine has no more than 90, 100, 110, 120, 130, 140, 150, 160, 170, 180, 190, or 200 amino acid residues.

In some embodiments, the methods further comprise (d) expressing a cytokine variant, wherein the variant comprises a non-native disulfide bond formed by mutating the two selected amino acid residues to cysteines; (e) administering the cytokine variant to a tumor-bearing animal; and (f) determining tumor growth (e.g., by measuring tumor volume) in the tumor-bearing animal. In some embodiments, administering the cytokine variant does not cause substantial toxicity to the animal. For example, the body weight of the animal administered with the cytokine variant does not drop significantly as compared to that of a reference animal (e.g., an animal administered with vehicle).

In some embodiments, the cytokine described herein is IL-21 (e.g., human IL-21). In some embodiments, the cytokine is IL-1, IL-2, IL-3, IL-4, IL-5, IL-6, IL-7, IL-8, IL-9, IL-10, IL-11, IL-13, IL-14, IL-15, IL-16, IL-17, IL-18, IL-19, IL-20, IL-22, IL-23, IL-24, IL-25, IL-26, IL-27, IL-28, IL-29, IL-30, IL-31, IL-32, IL-33, IL-35, or IL-36. In some embodiments, the cytokine is IL-2, IL-7, IL-10, IL-15, IL-21, IFNα, GM-CSF, or FLT-3.

In some embodiments, functional assays are performed to compare the one or more expressed protein variants (e.g., cytokine variants).

### EXAMPLES

The invention is further described in the following examples, which do not limit the scope of the invention described in the claims.

### Example 1. Design of IL-21 Variants by Introducing an Artificial Disulfide Bond

Interleukin-21 (IL-21) is a pleiotropic cytokine that is composed of four α-helical bundles and produced primarily by natural killer T (NKT) cells, T follicular helper (TFH) cells, and TH17 cells. IL-21 signals through IL-21R (IL-21 receptor) and utilizes the JAK-STAT, MAPK and PI3K pathways. Wild-type human IL-21 protein (SEQ ID NO: 1) has 162 amino acids, of which residues 1-24 constitutes the signal peptide.

To screen IL-21 variants with enhanced stability and different functional potencies (e.g., T cell proliferation and STAT-3 signaling), variants of wild-type IL-21 (SEQ ID NO: 2; without signal peptide) were designed by selectively mutating two spatially proximate residues to cysteines, according to the distance of the Cₐₗₚₕₐ atoms and directionalities of the C_{beta} atoms of the two residues within the 3D structure of IL-21 (e.g., PDB ID: 20QP). It is contemplated that the two newly introduced cysteines can form an artificial disulfide bond, thereby stabilizing IL-21 and/or changing its functional potency.

Sequences of the wild-type IL-21 and its variants are shown in the table below.

TABLE 1

| Name | Definition | Sequence |
|---|---|---|
| IL21-WT | Wild type | HKSSSQGQDR HMIRMRQLID IVDQLKNYVN |
| (SEQ ID NO: 2) | (without | DLVPEFLPAP EDVETNCEWS AFSCFQKAQL |
| | signal | KSANTGNNER IINVSIKKLK RKPPSTNAGR RQKHRLTCPS |
| | peptide) | CDSYEKKPPK EFLERFKSLL QKMIHQHLSS RTHGSEDS |
| IL21-C01 | Q8C- | HKSSSQGCDR HMIRMRQLID IVDQLKNYVN |
| (SEQ ID NO: 3) | T86C | DLVPEFLPAP EDVETNCEWS AFSCFQKAQL |
| | | KSANTGNNER IINVSIKKLK RKPPSCNAGR RQKHRLTCPS |
| | | CDSYEKKPPK EFLERFKSLL QKMIHQHLSS RTHGSEDS |
| IL21-C02 | I19C- | HKSSSQGQDR HMIRMRQLCD IVDQLKNYVN |
| (SEQ ID NO: 4) | K117C | DLVPEFLPAP EDVETNCEWS AFSCFQKAQL |
| | | KSANTGNNER IINVSIKKLK RKPPSTNAGR RQKHRLTCPS |
| | | CDSYEKKPPK EFLERFCSLL QKMIHQHLSS RTHGSEDS |
| IL21-C03 | V29C- | HKSSSQGQDR HMIRMRQLID IVDQLKNYCN |
| (SEQ ID NO: 5) | K110C | DLVPEFLPAP EDVETNCEWS AFSCFQKAQL |
| | | KSANTGNNER IINVSIKKLK RKPPSTNAGR RQKHRLTCPS |
| | | CDSYEKKPPC EFLERFKSLL QKMIHQHLSS RTHGSEDS |
| IL21-C04 | D31C- | HKSSSQGQDR HMIRMRQLID IVDQLKNYVN |
| (SEQ ID NO: 6) | S62C | CLVPEFLPAP EDVETNCEWS AFSCFQKAQL |
| | | KCANTGNNER IINVSIKKLK RKPPSTNAGR |
| | | RQKHRLTCPS CDSYEKKPPK EFLERFKSLL |
| | | QKMIHQHLSS RTHGSEDS |
| IL21-C05 | D31C- | HKSSSQGQDR HMIRMRQLID IVDQLKNYVN |
| (SEQ ID NO: 7) | A63C | CLVPEFLPAP EDVETNCEWS AFSCFQKAQL |
| | | KSCNTGNNER IINVSIKKLK RKPPSTNAGR RQKHRLTCPS |
| | | CDSYEKKPPK EFLERFKSLL QKMIHQHLSS RTHGSEDS |
| IL21-C06 | V33C- | HKSSSQGQDR HMIRMRQLID IVDQLKNYVN |
| (SEQ ID NO: 8) | K61C | DLCPEFLPAP EDVETNCEWS AFSCFQKAQL |
| | | CSANTGNNER IINVSIKKLK RKPPSTNAGR RQKHRLTCPS |
| | | CDSYEKKPPK EFLERFKSLL QKMIHQHLSS RTHGSEDS |
| IL21-C07 | V33C- | HKSSSQGQDR HMIRMRQLID IVDQLKNYVN |
| (SEQ ID NO: 9) | A63C | DLCPEFLPAP EDVETNCEWS AFSCFQKAQL |
| | | KSCNTGNNER IINVSIKKLK RKPPSTNAGR RQKHRLTCPS |
| | | CDSYEKKPPK EFLERFKSLL QKMIHQHLSS RTHGSEDS |
| IL21-C08 | F36C- | HKSSSQGQDR HMIRMRQLID IVDQLKNYVN |
| (SEQ ID NO: 10) | K106C | DLVPECLPAP EDVETNCEWS AFSCFQKAQL |
| | | KSANTGNNER IINVSIKKLK RKPPSTNAGR RQKHRLTCPS |
| | | CDSYECKPPK EFLERFKSLL QKMIHQHLSS RTHGSEDS |
| IL21-C09 | F36C- | HKSSSQGQDR HMIRMRQLID IVDQLKNYVN |
| (SEQ ID NO: 11) | K107C | DLVPECLPAP EDVETNCEWS AFSCFQKAQL |
| | | KSANTGNNER IINVSIKKLK RKPPSTNAGR RQKHRLTCPS |
| | | CDSYEKCPPK EFLERFKSLL QKMIHQHLSS RTHGSEDS |
| IL21-C10 | A39C- | HKSSSQGQDR HMIRMRQLID IVDQLKNYVN |
| (SEQ ID NO: 12) | E105C | DLVPEFLPCP EDVETNCEWS AFSCFQKAQL |
| | | KSANTGNNER IINVSIKKLK RKPPSTNAGR RQKHRLTCPS |
| | | CDSYCKKPPK EFLERFKSLL QKMIHQHLSS RTHGSEDS |
| IL21-C11 | A39C- | HKSSSQGQDR HMIRMRQLID IVDQLKNYVN |
| (SEQ ID NO: 13) | K107C | DLVPEFLPCP EDVETNCEWS AFSCFQKAQL |
| | | KSANTGNNER IINVSIKKLK RKPPSTNAGR RQKHRLTCPS |
| | | CDSYEKCPPK EFLERFKSLL QKMIHQHLSS RTHGSEDS |
| IL21-C12 | A39C- | HKSSSQGQDR HMIRMRQLID IVDQLKNYVN |
| (SEQ ID NO: 14) | F112C | DLVPEFLPCP EDVETNCEWS AFSCFQKAQL |
| | | KSANTGNNER IINVSIKKLK RKPPSTNAGR RQKHRLTCPS |
| | | CDSYEKKPPK ECLERFKSLL QKMIHQHLSS RTHGSEDS |
| IL21-C13 | Q56C- | HKSSSQGQDR HMIRMRQLID IVDQLKNYVN |
| (SEQ ID NO: 15) | K80C | DLVPEFLPAP EDVETNCEWS AFSCFCKAQL |
| | | KSANTGNNER IINVSIKKLC RKPPSTNAGR RQKHRLTCPS |
| | | CDSYEKKPPK EFLERFKSLL QKMIHQHLSS RTHGSEDS |

The IL-21 variants fused with a His tag (SEQ ID NO: 22) were expressed in Expi293 and CHO-S cells. The expressed proteins were purified by affinity chromatography using a Ni-HisTrap™ column (Cytiva, Cat #: 17371206), and size exclusive chromatography (SEC) using a Superdex® 75 column (Cytiva, Cat #: 29148721). The purified proteins were also analyzed by sodium dodecyl sulfate-polyacrylamide gel electrophoresis (SDS-PAGE).

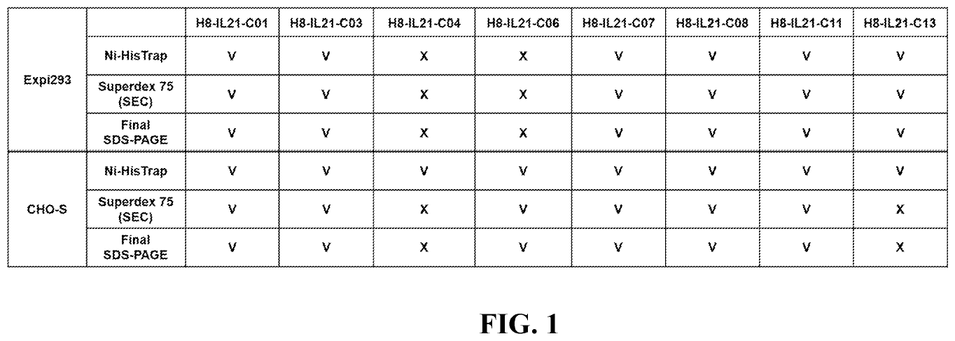

As shown in FIG. 1, the results showed that H8-IL21-C04, H8-IL21-C06 and H8-IL21-C13 can express in either Expi293 or CHO-S cells, while H8-IL21-C02, H8-IL21-C05, H8-IL21-C09, H8-IL21-C10 and H8-IL21-C12 showed no expression in Expi293 or CHO-S cells. By contrast, H8-IL21-C01, H8-IL21-C03, H8-IL21-C07, H8-IL21-C08, and H8-IL21-C011 can express in both Expi293 or CHO-S cells.

### Example 2. Determination of Potency on Induction of STAT-3 Phosphorylation and T Cell Proliferation

The potency of IL-21 variants on T cell responses were determined by STAT-3 phosphorylation and T cell proliferation. Specifically, for STAT-3 phosphorylation assays, CD3+ T cells were isolated from a hPBMC (human peripheral blood mononuclear cell) donor, according to the manufacturing protocols. The CD3+ T cells were activated by CD3/CD28 Dynabeads® at 1:4 cell-to-beads ratio for 3 days. Activated T cells were rested for 24 hours, and 5×10⁴/well of CD3+ T cells were then incubated with His-tagged IL-21 variants at indicated concentrations (2 nM, 5-fold serial dilutions, 6 spots) for 30 minutes at 37° C. After the incubation, the CD3+ T cells were harvested and permeabilized. The cells were further stained with 2 μl pSTAT3 (Y705)-PE antibody and then analyzed by flow cytometry. For T cell proliferation assays, CD3+ T cells were isolated from a hPBMC donor according to manufacturing protocols. The CD3+ T cells were activated by CD3/CD28 Dynabeads® at a 1:4 cell-to-beads ratio for 3 days. Activated T cells were rested for 24 hours, 5×10⁴/well of CD3+ T cells were then incubated with His-tagged IL-21 variants at indicated concentrations, in the presence of pre-coated 1 μg/ml of anti-CD3 antibody for 3 days. After the incubation, T cell proliferation was determined by the CellTiter-Glo® Luminescent Cell Viability Assay (Promega, Cat #: G7573).

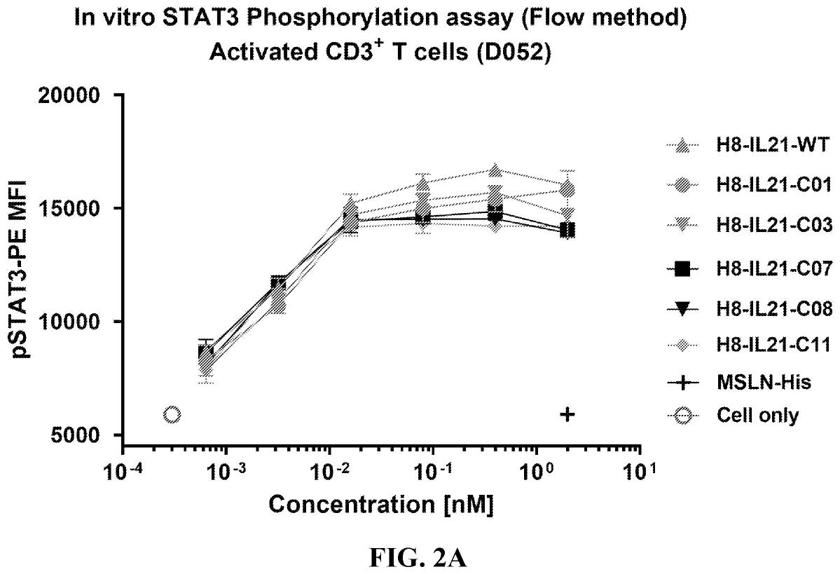

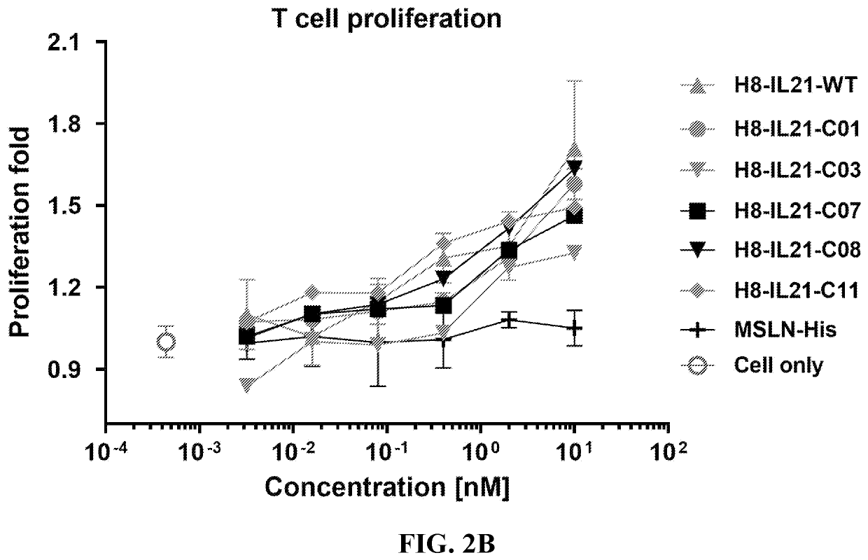

As shown in FIG. 2A, all IL-21 variants showed relatively lower potency on induction of STAT-3 phosphorylation with minor differences. As showed in FIG. 2B, H8-IL21-C11 showed the highest potency on induction of T cell proliferation. H8-IL21-C03 showed relatively weaker capability on induction of T cell proliferation. H8-IL21-C01, H8-IL21-C07 and H8-IL21-C08 showed a similar efficacy on induction of T cell proliferation as compared to the wild-type control (H8-IL21-WT). His-tagged mesothelin extracellular domain (MSLN-His) was used as a negative control.

Based on the protein expression and T cell response results of IL-21 variants, the IL-21 variants IL21-C01 (SEQ ID NO: 3), IL21-C03 (SEQ ID NO: 5), IL21-C07 (SEQ ID NO: 9), IL21-C08 (SEQ ID NO: 10), and IL21-C11 (SEQ ID NO: 13) were selected for subsequent experiments.

### Example 3. Expression and Purification of HSA-Fused IL-21 Variants

To enhance the stability of IL-21 variants, plasmids with a human promoter uHS or HS (UCOE® Single Expression Puromycin Vector CET 1019 HS-puro-Scel; Merck, Cat #: UCOE01) encoding wild-type IL-21 (SEQ ID NO: 2) and selected IL-21 variants (IL21-C01, IL21-C03, IL21-C07, and IL21-C08) fused with HSA (human serum albumin) were constructed. Expi293 cells were transfected to express the HSA-fused IL-21 HSA-IL21-WT (SEQ ID NO: 17), and its variants HSA-IL21-C01 (SEQ ID NO: 18), HSA-IL21-C03 (SEQ ID NO: 19), HSA-IL21-C07 (SEQ ID NO: 20), and HSA-IL21-C08 (SEQ ID NO: 21). The HSA-fused IL-21 variants were purified and characterized as follows.

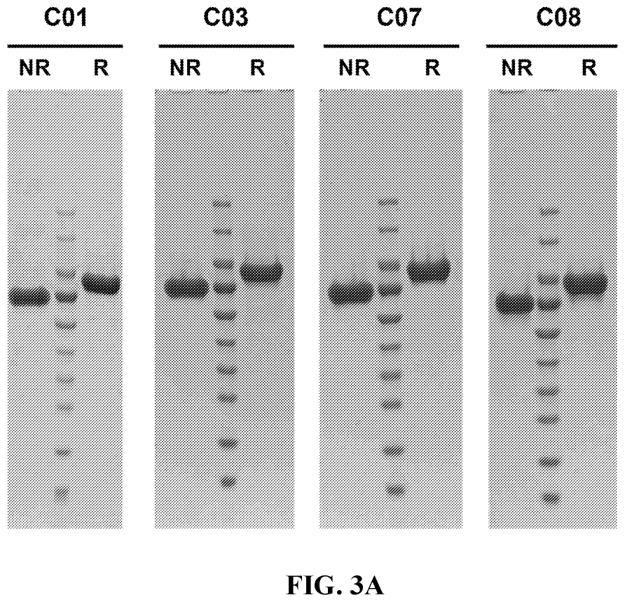

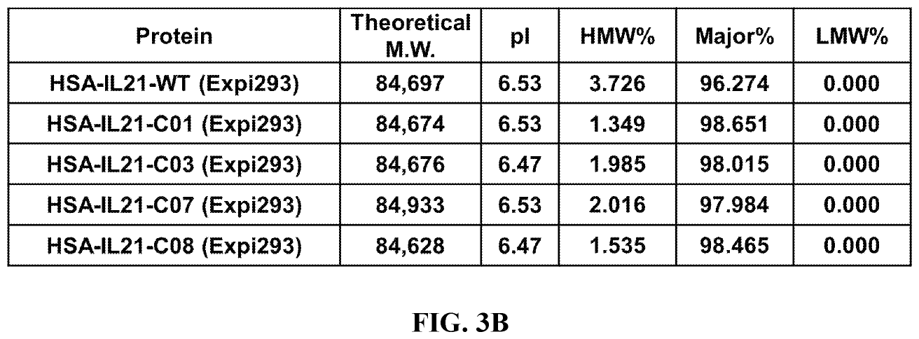

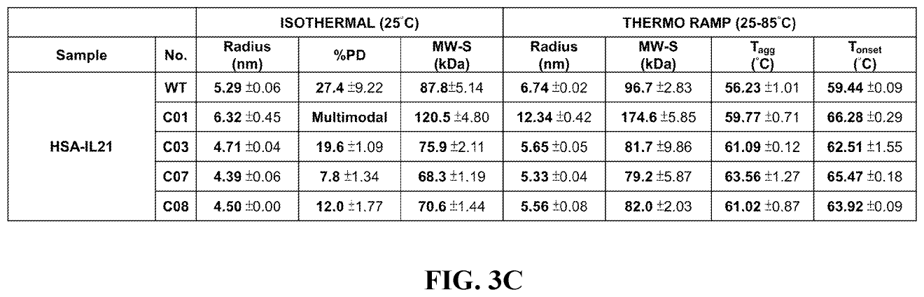

First, the purity of HSA-fused IL-21 variants were analyzed by SDS-PAGE and HPLC-SEC (high-performance liquid chromatography-size exclusion chromatography), respectively. As shown in FIG. 3A, all of the selected HSA-fused IL-21 variants showed no aggregation or small fragmentation by non-reducing (NR) or reducing (R) SDS-PAGE. As shown in FIG. 3B, the HPLC-SEC analysis results demonstrated that the purity (as indicated by “Major %”) of all the selected HSA-fused IL-21 variants were above 95%.

In addition, the aggregation (T_{agg}) and onset (Tₒₙₛₑₜ) temperatures of HSA-fused IL-21 variants were analyzed by dynamic and static light scattering (DLS/SLS). As showed in FIG. 3C, compared to HSA-IL21-WT, all the selected HSA-fused IL-21 variants showed significantly increased T_{agg} and Tₒₙₛₑₜ temperatures. The results indicated that the selected IL-21 variants had better stability than wild-type IL-21.

### Example 4. Determination of Potency of HSA-Fused IL-21 Variants on Induction of STAT-3 Phosphorylation and T Cell Proliferation

The potency of HSA-fused IL-21 variants on T cell responses were determined by STAT-3 phosphorylation and T cell proliferation. Specifically, for STAT-3 phosphorylation assays, CD8+ T cells were isolated from a hPBMC donor, according to the manufacturing protocols. The CD8+ T cells were activated by CD3/CD28 Dynabeads® at 1:5 cell-to-beads ratio for 3 days. Activated T cells were rested for 24 hours, and 5×10⁴/well of CD8+ T cells were then incubated with His-tagged HSA-fused IL-21 variants at indicated concentrations (1 nM, 20-fold serial dilutions, 4 spots) for 30 minutes at 37° C. After the incubation, the CD8+ T cells were harvested and permeabilized. The cells were further stained with 2 μl pSTAT3 (Y705)-PE antibody and then analyzed by flow cytometry. For T cell proliferation assays, CD8+ T cells were isolated from a hPBMC donor according to manufacturing protocols. The CD8+ T cells were activated by CD3/CD28 Dynabeads® at a 1:8 cell-to-beads ratio for 3 days. Activated T cells were rested for 24 hours, 5×10⁴/well of CD8+ T cells were then incubated with His-tagged HSA-fused IL-21 variants at indicated concentrations (10 nM, 5-fold serial dilutions, 6 spots), in the presence of pre-coated 1 μg/ml of anti-CD3 antibody for 6 days. After the incubation, T cell proliferation was determined by the CellTiter-Glo® Luminescent Cell Viability Assay (Promega, Cat #: G7573).

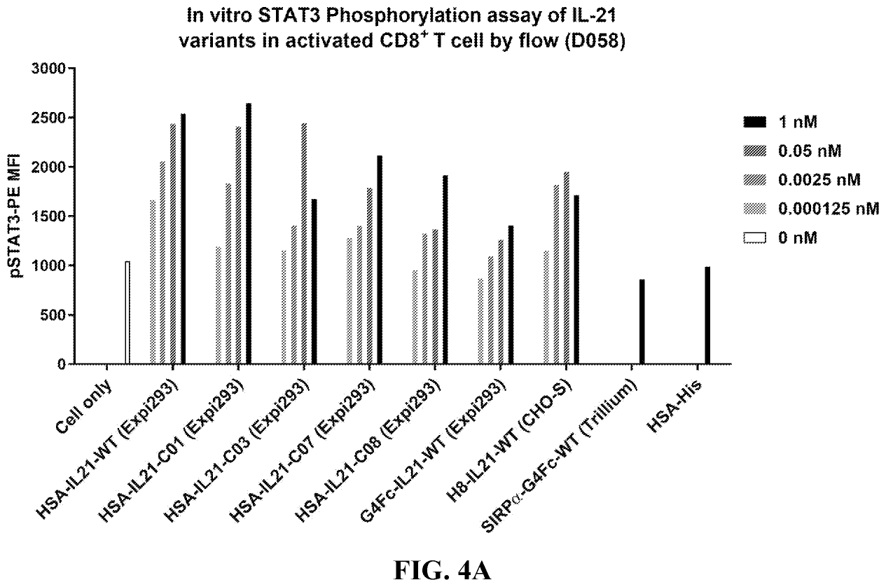

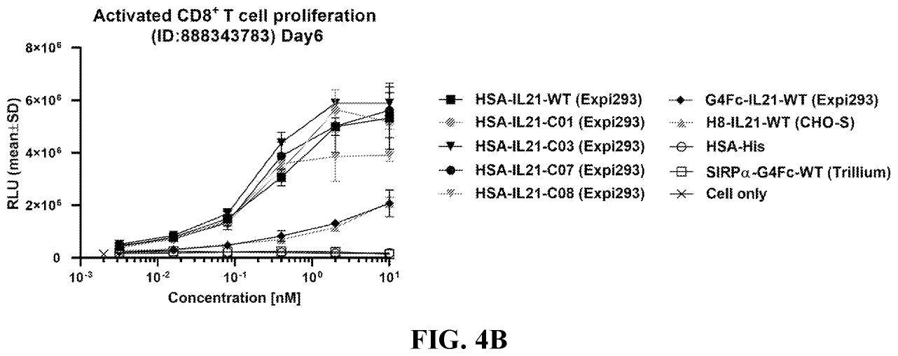

As shown in FIG. 4A, HSA-IL21-C03, HSA-IL21-C07, and HSA-IL21-C08 showed a relatively weaker potency on induction of STAT-3 phosphorylation than HSA-IL21-WT. HSA-IL21-C01 showed a similar potency on induction of STAT-3 phosphorylation as compared to HSA-IL21-WT. As showed in FIG. 4B, HSA-L21-C03 showed the highest potency on induction of T cell proliferation than HSA-IL21-WT and other HSA-fused IL-21 variants. However, HSA-IL21-C08 showed a relatively weaker potency on induction of T cell proliferation than HSA-IL21-WT, but the difference was minor. His-tagged HSA (HSA-His), MSLN-His, and SIRPα-G4Fc (Trillium) (or SIRPα-G4Fc-WT (Trillium); SEQ ID NO: 23) were used as negative controls.

The results also showed that IgG4 Fc fused wild-type IL-21 (G4Fc-IL21-WT; SEQ ID NO: 16) and H8-IL21-WT expressed by CHO-S cells (H8-IL21-WT_CHOS) exhibited a relatively weaker potency on induction of STAT-3 phosphorylation and T cell proliferation, as compared to HSA-fused wild-type IL-21 and its variants expressed in Expi293 cells.

### Example 5. Determination of Potency of HSA-Fused IL-21 Variants on Induction of NK-92 Cell Proliferation

The potency of HSA-fused IL-21 variants on induction of NK-92 cell proliferation was determined by CellTiter-Glo® Luminescent Cell Viability Assay (Promega, Cat #: G7573). Specifically, NK-92 cells were starved in a medium without IL-2 supplement for 24 hours. After the starvation, 5×10⁴/well of NK-92 cells were incubated with HSA-IL-21 variants at indicated concentrations (100 nM, 10-fold serial dilutions, 8 spots) in a complete medium (75% MEM-α with ribo- and deoxyribonucleosides (Gibco, Cat #: 12571-048)+12.5% horse serum+12.5% FBS+150 U/mL IL-2) for another 24 hours. The proliferation was determined by CellTiter-Glo® Luminescent Cell Viability Assay.

The EC50 value of each HSA-fused IL-21 variants are shown in the table below.

TABLE 2

| | HSA-IL21-WT | HSA-IL21-C01 | HSA-IL21-C03 | HSA-IL21-C07 | HSA-IL21-C08 |
|---|---|---|---|---|---|
| | (HS, Expi293) | (HS, Expi293) | (HS, Expi293) | (HS, Expi293) | (HS, Expi293) |
| EC50 (nM) | 0.0877 | 0.1454 | ~0.0784 | ~0.1084 | 0.1166 |

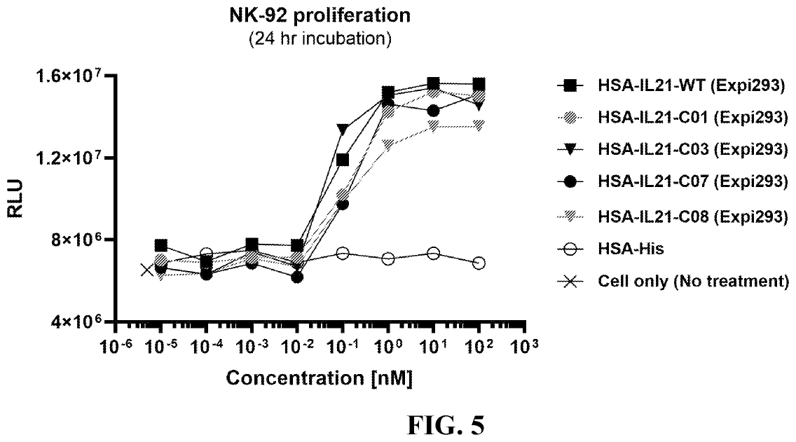

As shown in FIG. 5 and the above table, HSA-IL21-C03 showed a better potency on induction of NK-92 cell proliferation than HSA-IL21-WT. HSA-IL21-C08 showed the lowest potency among the HSA-fused IL-21 variants on induction of NK-92 cell proliferation. HSA-His Protein (Acro) was used as a negative control.

### Example 6. Determination of Potency of HSA-Fused IL-21 Variants on Relieving Treg-Mediated T Cell Suppression

The potency of HSA-fused IL-21 variants on Treg function was determined by Treg-mediated T cell suppression assay.

Specifically, CD4⁺CD25⁺CD127low Treg cells were isolated from a hPBMC donor according to manufacturing protocols. The Treg cells were activated by CD3/CD28 Dynabeads® at a 1:10 cell-to-beads ratio for 4 days. After activation, CD8⁺ T cells were isolated from another hPBMC donor according to manufacturing protocols, and then stained with 1 μM of CFSE (carboxyfluorescein succinimidyl ester) dye. Treg and CFSE-labeled CD8⁺ T cells were incubated at a 1:4 cell-to-cell ratio, and then treated with or without HSA-fused IL-21 and its variants at indicated concentrations for 3 Days. Afterwards, the cells were harvested and stained with PE-anti-CD8 antibodies. The T cell proliferation were determined by the percentage of CD8⁺CFSE^{low} cells.

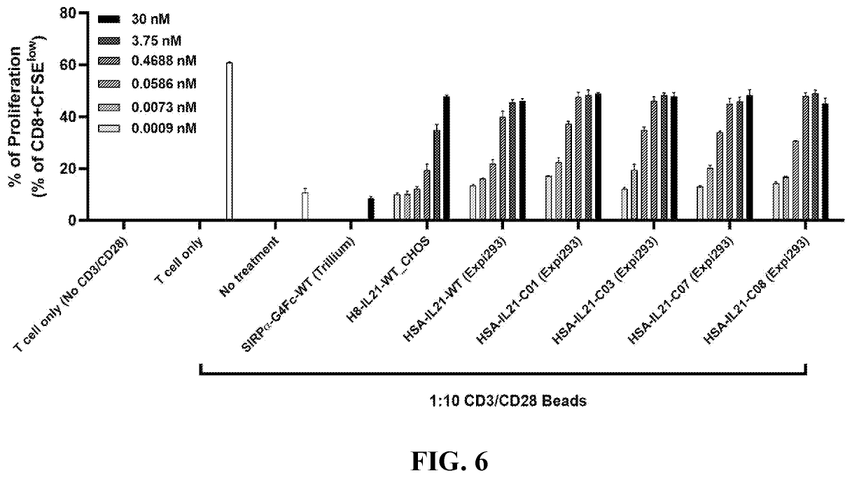

As shown in FIG. 6, compared to HSA-IL21-WT, all of the selected HSA-fused IL-21 variants (HSA-IL21-C01, HSA-IL21-C03, HSA-IL21-C07, and HSA-IL21-C08) showed a significant enhancement (e.g., at about 0.0586 nM) on relieving Treg-mediated T cell suppression. In particular, HSA-IL21-C08 showed less potency on relieving Treg-mediated T cell suppression than other HSA-fused IL-21 variants. HSA-IL21-C01, HSA-IL21-C03, and HSA-IL21-C07 exhibited a similar efficacy on Treg function. SIRPα-G4Fc-WT (Trillium) was used as a negative control.

The results also showed that HSA-IL21-WT exhibited a higher potency on relieving Treg-mediated T cell suppression than H8-IL21-WT (SEQ ID NO: 24).

### Example 7. Determination of Potency of HSA-Fused IL-21 Variants on Induction of Primary NK Cell Cytotoxicity

The potency of HSA-fused IL-21 variants on induction of primary NK cell cytotoxicity was determined by DELFIA® EuTDA Cytotoxicity Reagents (PerkinElmer). Specifically, primary NK cells were isolated from a hPBMC donor according to manufacturing protocols, and then incubated with or without HSA-fused IL-21 and its variants at indicated concentrations for 24 hours. Before incubated with primary NK cells, K-562 tumor cells were treated with 20 ng/ml of IFN-γ for 24 hours and then labeled with a fluorescence enhancing ligand BATDA (bis(acetoxymethyl) 2,2′: 6′,2″-terpyridine-6,6″-dicarboxylate) according to DELFIA® EuTDA Cytotoxicity Reagents (PerkinElmer) manufacturing protocols. Next, effector cells (primary NK cell) and target cells (BATDA-labeled K-562) were co-incubated at a cell-to-cell ratio of 4:1 in a 96-well U-type cell culture plate, in the presence of 1-100 nM HSA-fused IL-21 variants for 4 hours at 37° C. Cell supernatant was carefully collected from the assay plate so as not to disturb the cells. 20 μl of cleared supernatant sample was mixed with 200 μl of europium solution in the provided DELFIA® strip plate. Signal was detected using DELFIA® time-resolved fluorescence (TRF) settings on a compatible plate reader (excitation 340 nm and emission at 615 nm). The percentage of specific release was determined by following formula:

$$( Experimental Release - Spontaneous Release ) / ( Maximum Release - Spontaneous Release ) \times 100.$$

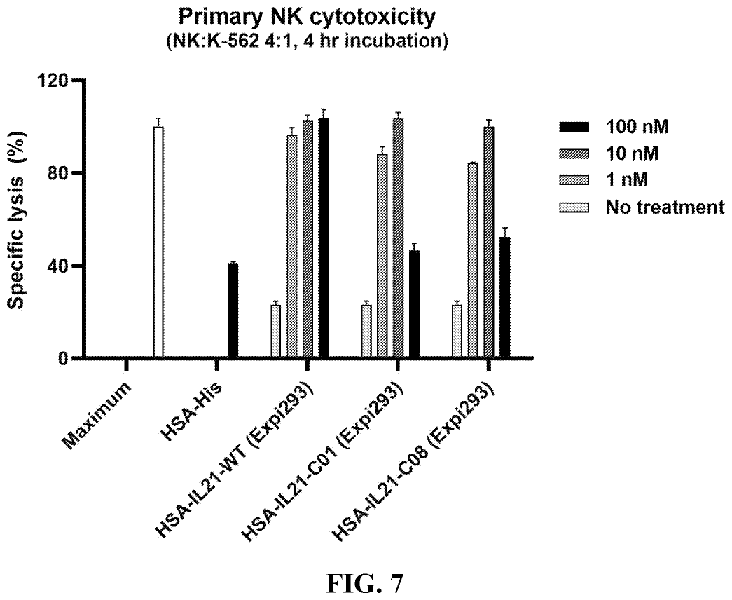

As shown in FIG. 7, HSA-IL21-C01 and HSA-IL21-C08 showed less potency on induction of primary NK cell cytotoxicity than HSA-IL21-WT. The results indicate that the selected IL-21 variants can induce specific lysis of K-562 tumor cells mediated by primary NK cells.

Based on the characterization and in vitro assay results above, HSA-IL21-C01 and HSA-IL21-C08 were lead candidates, and they were selected for subsequent experiments.

### Example 8. Evaluation of the Antitumor Efficacy and In Vivo Toxicity of HSA-Fused IL-21 Variants

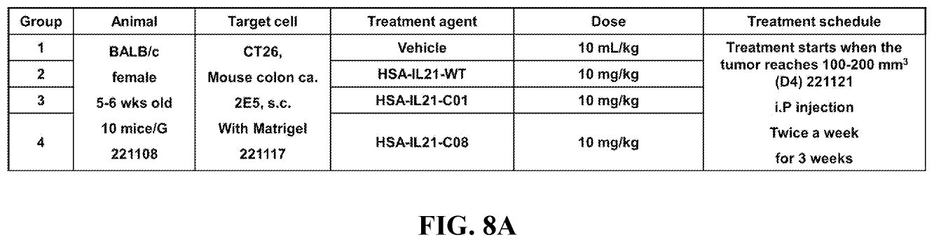

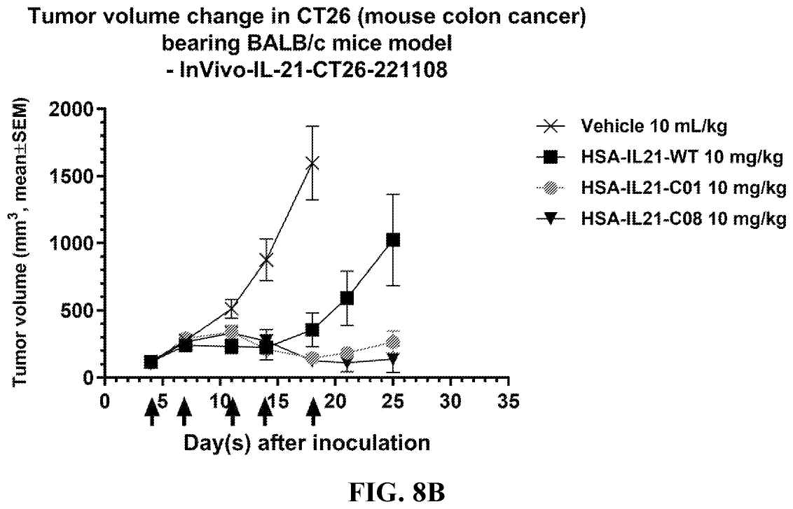

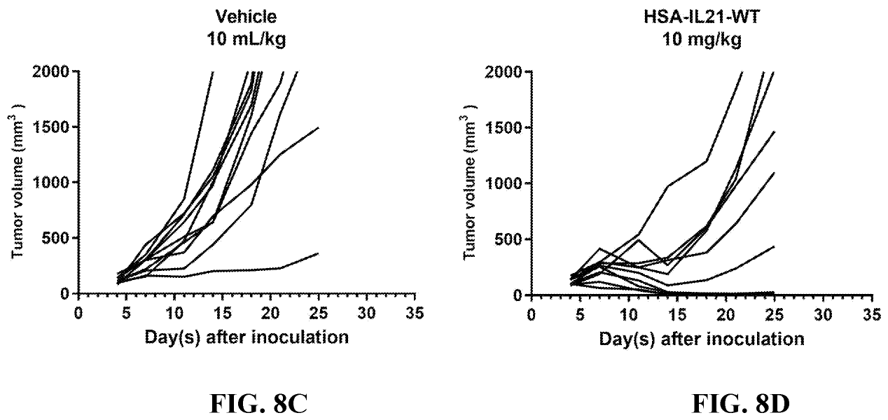

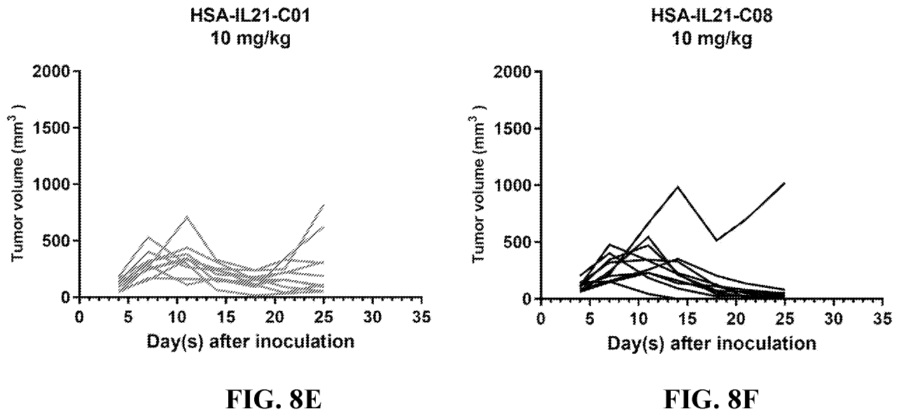

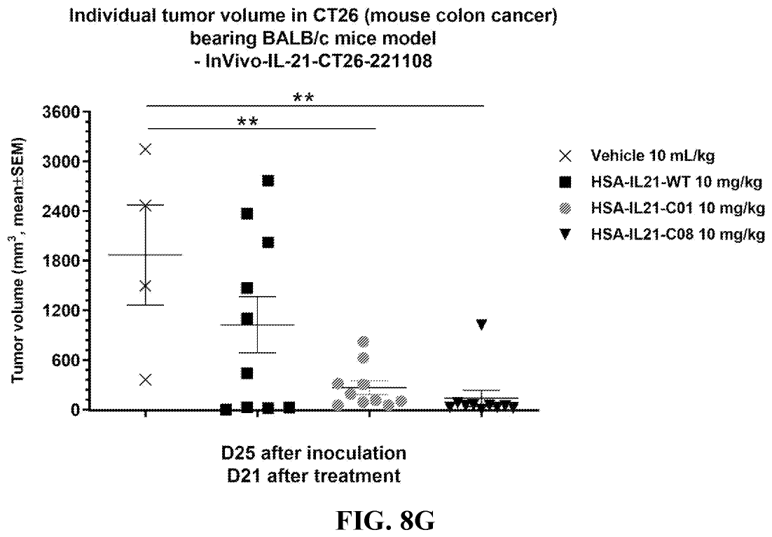

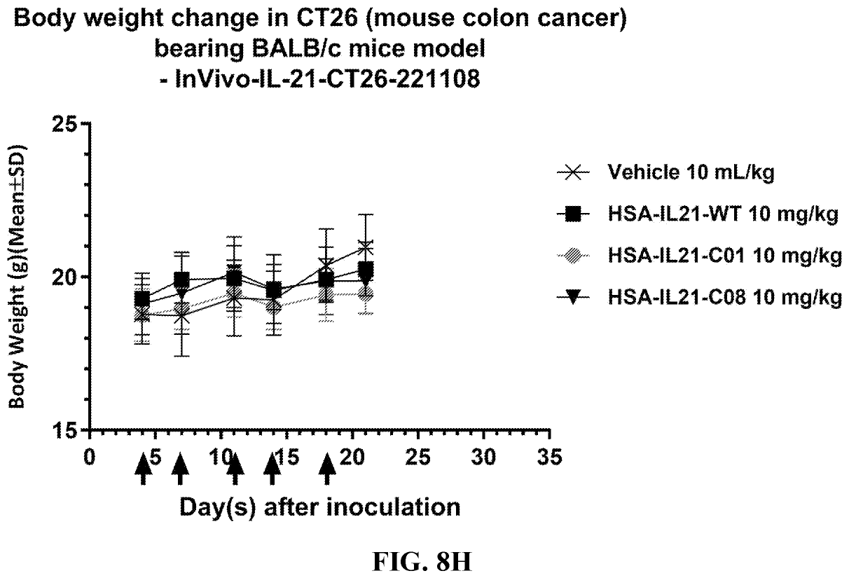

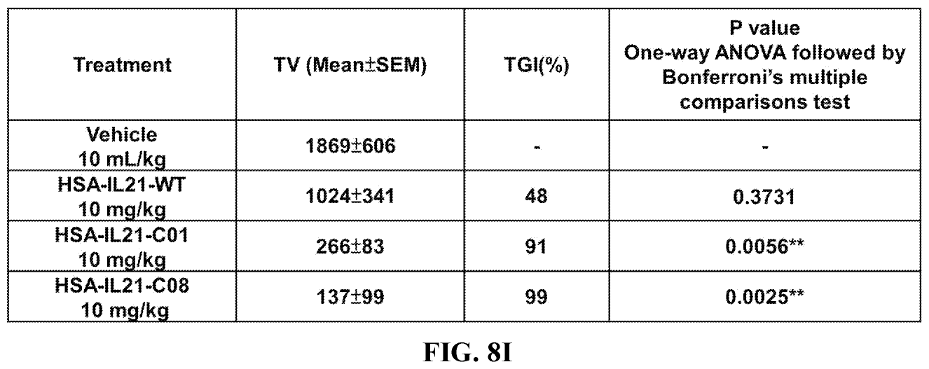

The anti-tumor efficacy of HSA-fused IL-21 and its variants were evaluated in a CT26 tumor-bearing BALB/c mouse model. The treatment plan and dosing schedule are shown in FIG. 8A. Specifically, 5-6 weeks old BALB/c mice were selected and subcutaneously (s.c.) inoculated with 2× 10⁵ mouse colon cancer cell CT26. When the tumor volume reached about 100-200 mm³ (after 4 days), the mice were randomly placed into 3 treatment groups and 1 control group (10 mice per group). The treatment group mice were intraperitoneally (i.p.) injected with 10 mg/kg HSA-IL21-WT (G2), HSA-IL21-C01 (G3), or HSA-IL21-C08 (G4). The control group mice (G1) were injected with 10 mL/kg vehicle (isotonic sodium chloride solution). The treatment started on the grouping day (Day 0), and the injections were performed twice a week (for a total of 3 weeks).

As shown in FIG. 8B, compared to HSA-IL21-WT, HSA-IL21-C01 and HSA-IL21-C08 showed significant tumor growth inhibition. Individual tumor growth curves and the tumor volume of each mouse on Day 21 are shown in FIGS. 8C-8F and FIG. 8G, respectively. The results indicate that HSA-IL21-C01 and HSA-IL21-C08 induced remarkable tumor shrinkage in the CT26 syngeneic mouse model. Body weight change results in FIG. 8H suggest that treatment of HSA-fused IL-21 and its variants did not induce significant toxicity on CT26 syngeneic mice. The overall summary table of percentage of tumor growth inhibition (% TGI) is shown in FIG. 8I. The results show that the TGI % of HSA-IL21-C01 and HSA-IL21-C08 is 91% and 99%, respectively, in the CT26 syngeneic mouse model. The corresponding P values were determined to be less than 0.01.

### Example 9. Determination of Potency of HSA-Fused IL-21 Variants on Relieving Treg-Mediated T Cell Suppression

CD4⁺CD25⁺CD127low Treg cells were isolated from a hPBMC donor according to manufacturing protocols. The Treg cells were activated by CD3/CD28 Dynabeads® (Thermo Fisher, Cat #: 11132D) at a 1:10 cell-to-beads ratio and treated with or without HSA-fused IL-21 and its variants at indicated concentrations for 4 days. After the 4-day treatment, the CD3/CD28 Dynabeads® and IL-21-containing medium were discarded. In addition, CD8⁺ T cells were isolated from another hPBMC donor according to manufacturing protocols, and then stained with 1 μM of CFSE (Thermo Fisher, Cat #: C34554) dye. The Treg and CFSE-labeled CD8⁺ T cells were incubated at a 1:8 cell-to-cell ratio for 6 days. Afterwards, the cells were harvested and stained with PE-anti-CD8 antibodies (BioLegend, Cat #: 980902). The T cell proliferation was determined by the percentage of CD8 CFSE^{low} cells.

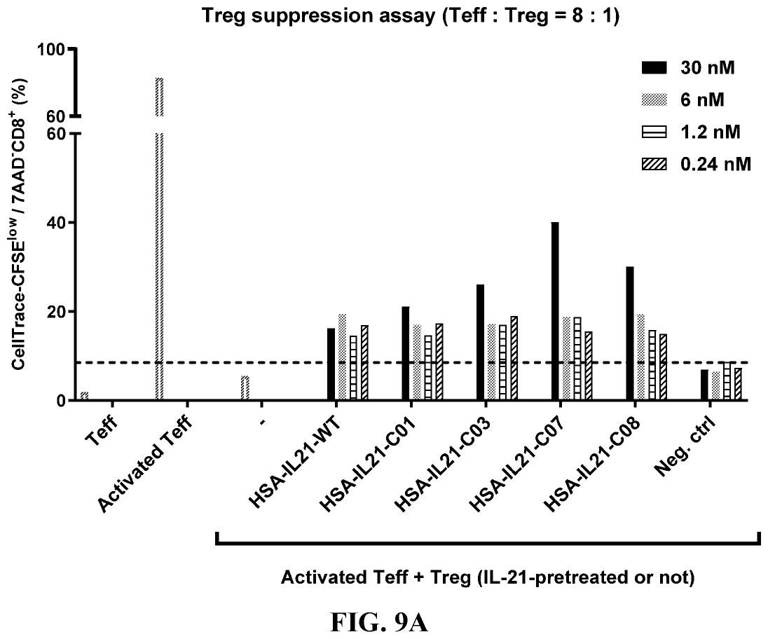

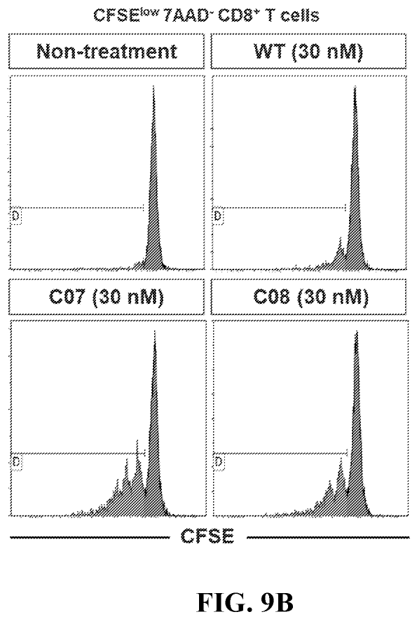

As shown in FIGS. 9A-9B, all IL-21 variants could relieve CD8⁺ T cell suppression-inducing by Treg cells. Compared to HSA-IL21-WT, all of the selected HSA-fused IL-21 variants (HSA-IL21-C01, HSA-IL21-C03, HSA-IL21-C07, and HSA-IL21-C08) showed a significant enhancement on relieving Treg-mediated T cell suppression.

### Example 10. Determination of Potency of HSA-Fused IL-21 Variants on Induction of Primary NK Cell Cytotoxicity

The potency of HSA-fused IL-21 variants to induce NK cytotoxic activity on K-562 cells was detected as described in Example 7.

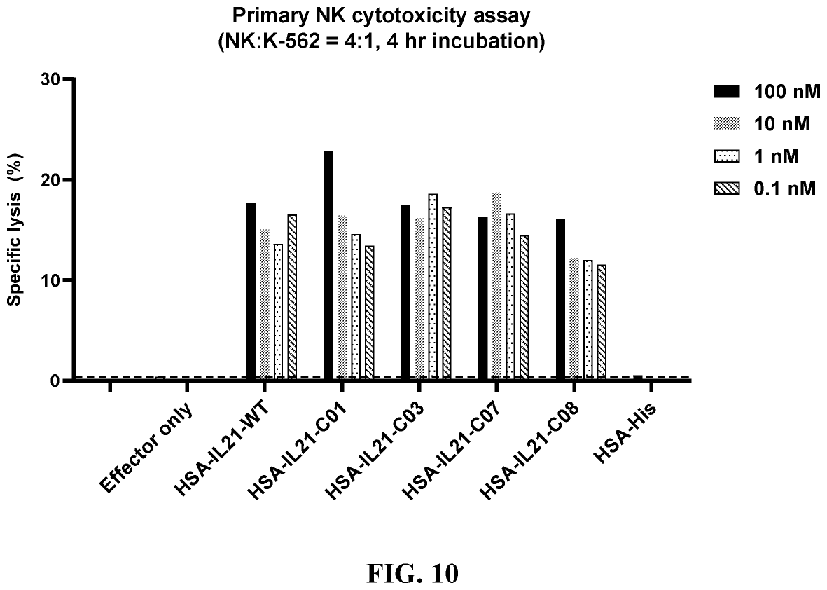

As shown in FIG. 10, IL21-WT and variants showed the potency on induction of NK cell cytotoxicity. All of the selected HSA-fused IL-21 variants (HSA-IL21-C01, HSA-IL21-C03, HSA-IL21-C07, and HSA-IL21-C08) showed a similar potency on induction of primary NK cell cytotoxicity than HSA-IL21-WT. The results indicate that the selected IL-21 variants could induce specific lysis of K-562 tumor cells mediated by primary NK cells.

### Example 11. Evaluation of the Antitumor Efficacy and Memory Effect of HSA-Fused IL-21 Variants

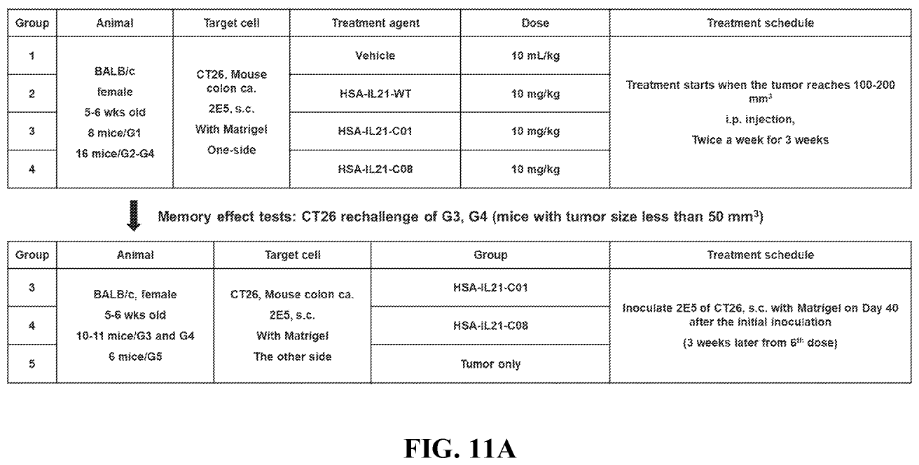

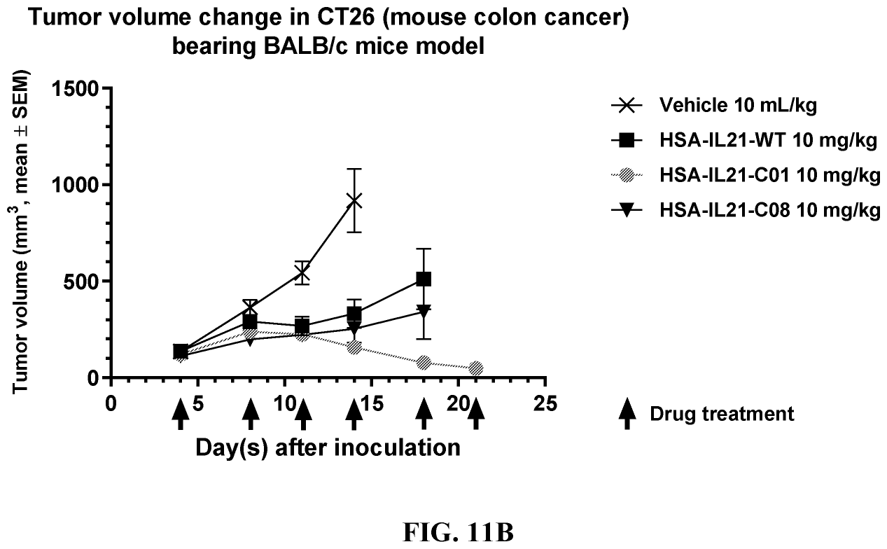

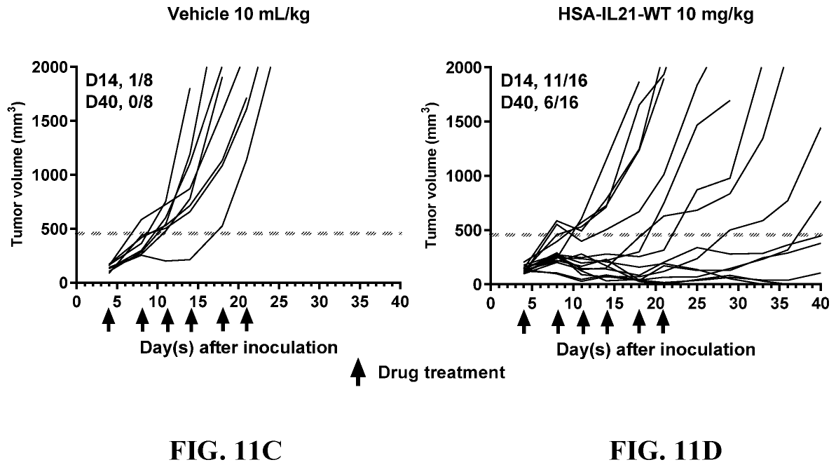

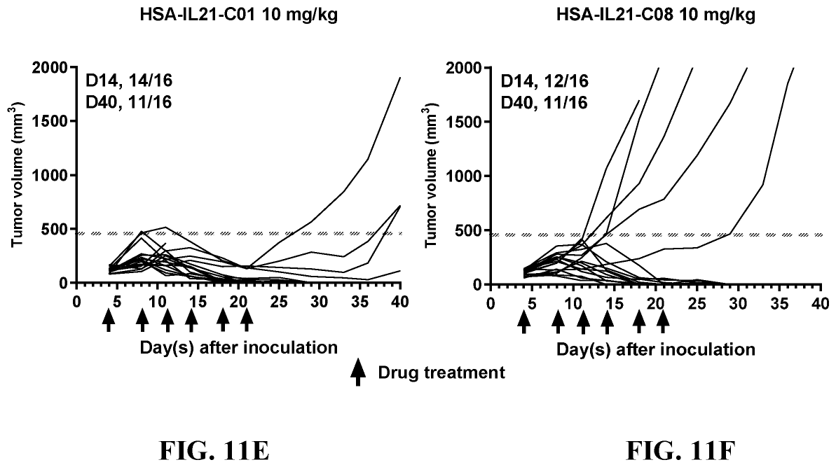

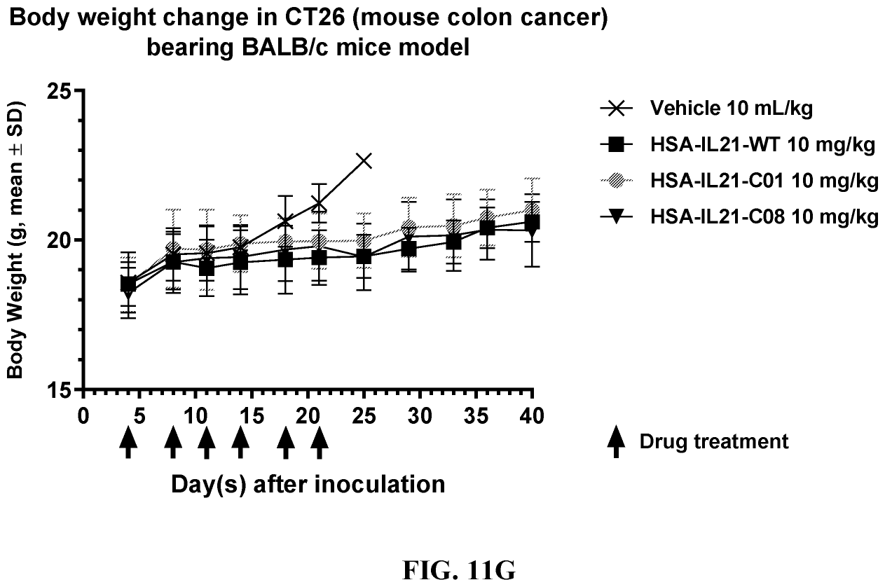

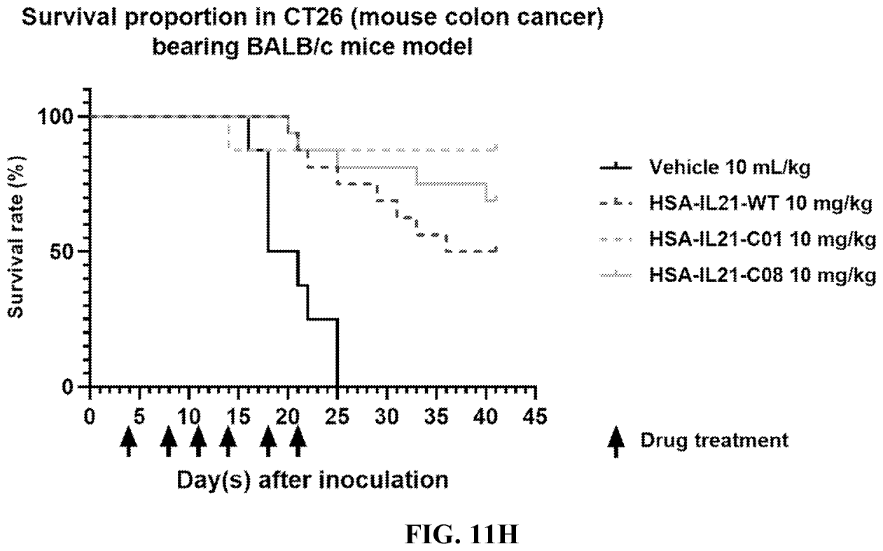

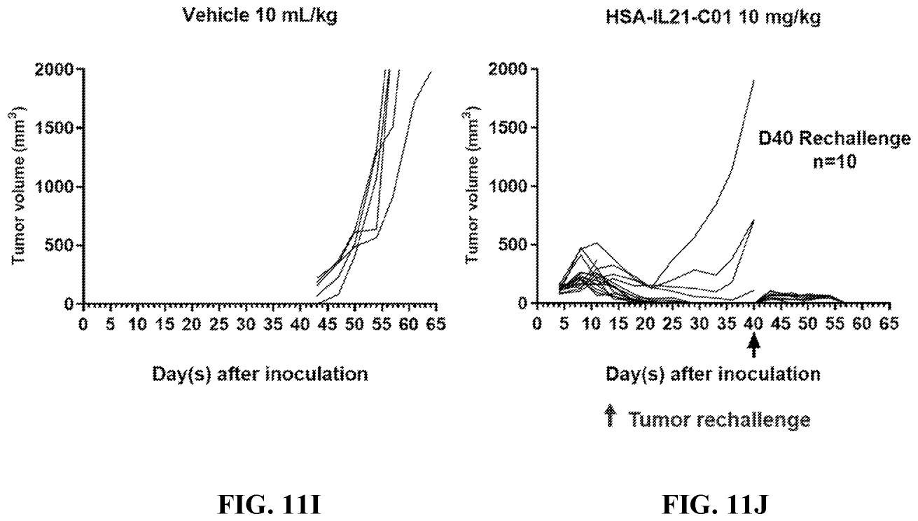

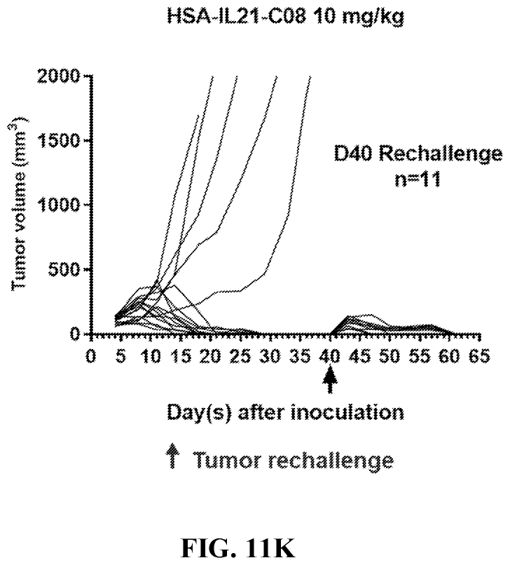

The anti-tumor efficacy and memory effect of HSA-fused IL-21 and its variants were evaluated in a CT26 tumor-bearing BALB/c mouse model. The treatment plan and dosing schedule are shown in FIG. 11A. To determine the anti-tumor efficacy, 5-6 weeks old BALB/c mice were selected and subcutaneously (s.c.) inoculated with 2× 10⁵ mouse colon cancer cell CT26 (on one side of all mice). When the tumor volume reached about 100-200 mm3 (4 days after tumor inoculation), the mice were randomly placed into 3 treatment groups (16 mice per group) and 1 control group having 8 mice. The treatment group mice were intraperitoneally (i.p.) injected with 10 mg/kg HSA-IL21-WT (G2), HSA-IL21-C01 (G3), or HSA-IL21-C08 (G4). The control group mice (G1) were injected with 10 mL/kg vehicle (isotonic sodium chloride solution). The treatment started on the grouping day (Day 4), and the injections were performed twice a week (for a total of 3 weeks).

After 6 administrations, the mice were rested (without treatment after Day 21) and observed for tumor growth. On Day 40 after the initial inoculation, the CT26 tumor-bearing mice in G3 and G4 groups whose tumor size was below 50 mm³ were selected for tumor rechallenge to determine the memory effect of HSA-fused IL-21 and its variants. 10 out of 16 mice in the original G3 group and 11 out of 16 mice in the original G4 group were subcutaneously (s.c.) inoculated with 2×10⁵ CT26 cells on the other side of the mice. The new control group mice (G5, 6 mice) were subcutaneously (s.c.) inoculated with 2× 105 CT26 cells as the rechallenge control group.

As shown in FIG. 11B, compared to HSA-IL21-WT, HSA-IL21-C01 and HSA-IL21-C08 showed significant tumor growth inhibition. Individual tumor growth curves of each mouse in the G1-G4 groups are shown in FIGS. 11C-11F, respectively. In these figures, the dashed line indicates half the value of the mean tumor volume of the vehicle group (G1) on Day 14, which was 458 mm³. The ratios after “D14” and “D40” show the number of mice with a tumor size under 458 mm³ on Day 14 and Day 40 after tumor inoculation, respectively, relative to the total number of mice in the original group. The results indicate that HSA-IL21-C01 and HSA-IL21-C08 induced remarkable tumor shrinkage in the CT26 syngeneic mouse model. Body weight change results in FIG. 11G suggest that treatment of HSA-fused IL-21 and its variants did not induce significant toxicity on CT26 syngeneic mice. The mouse survival curves in FIG. 11H show that HSA-IL21-C01 and HSA-IL21-C08 significantly elongated the mice survival as compared to HSA-IL21-WT. As shown in FIGS. 11I-11K, HSA-IL21-C01 and HSA-IL21-C08 showed a memory effect in the CT26-bearing BALB/c syngeneic mouse model. The results indicate that IL-21 signaling is important for the generation of tumor-specific memory responses to tumor rechallenge and the tested HSA-fused IL-21 variants could induce robust tumor-specific memory responses.

### Example 12. Expression and Purification of Fc-Fused IL-21 Variants

To test other formats of IL-21 variants, heterodimeric Fc-fused IL-21 variants were designed. Specifically, each heterodimeric Fc-fused IL-21 variant contains two polypeptide chains, i.e., a first polypeptide chain (or a “hole chain”) comprising from N-terminus to C-terminus: a human IgG4 hinge region (SEQ ID NO: 31), a human IgG4 Fc region with hole mutations (SEQ ID NO: 33), a linker peptide (SEQ ID NO: 34), and an IL-21 variant (e.g., any one of the IL-21 variants described above), and a second polypeptide chain (or a “knob chain”) comprising from N-terminus to C-terminus: a human IgG4 hinge region (SEQ ID NO: 31), and a human IgG4 Fc region with knob mutations (SEQ ID NO: 32). Plasmids expressing heterodimeric Fc-fused IL-21 and its variants were constructed. Expi293 cells were transfected to express the following molecules: heterodimeric Fc-IL21-WT (hole chain: SEQ ID NO: 35; knob chain: SEQ ID NO: 29), heterodimeric Fc-IL21-C01 (hole chain: SEQ ID NO: 36; knob chain: SEQ ID NO: 29), heterodimeric Fc-IL21-C03 (hole chain: SEQ ID NO: 37; knob chain: SEQ ID NO: 29), heterodimeric Fc-IL21-C07 (hole chain: SEQ ID NO: 38; knob chain: SEQ ID NO: 29), and heterodimeric Fc-IL21-C08 (hole chain: SEQ ID NO: 39; knob chain: SEQ ID NO: 29).

The following reasons were considered for generating the heterodimeric Fc-fused IL-21 variants. First, some reports showed that HSA-fused proteins may have precipitation issues at 37° C. and in acidic pH conditions. Second, it was contemplated that heterodimeric Fc-fused proteins may be more stable than HSA-fused proteins. Finally, preliminary data showed that heterodimeric Fc-IL21-WT exhibited a stronger IL-21 signaling-inducing activity than homodimeric Fc-IL21-WT (e.g., G4Fc-IL21-WT, which contains two identical polypeptide chains with the amino acid sequence of each chain set forth in SEQ ID NO: 16).

The purified heterodimeric Fc-IL21-WT, heterodimeric Fc-IL21-C01, heterodimeric Fc-IL21-C03, heterodimeric Fc-IL21-C07, and heterodimeric Fc-IL21-C08 were used for subsequent experiments.

### Example 13. Determination of Potency of Heterodimeric Fc-Fused IL-21 Variants on Induction of IL-21-STAT3 Reporter Signaling

IL-21 reporter assays were performed to test the IL-21-phspho STAT3 (pSTAT3)-signaling-inducing activity of heterodimeric Fc-fused IL-21 and its variants. Specifically, 2.8× 10⁵ cells/mL of HEK-Blue™ IL-21 (InvivoGen, Cat #: hkb-il21) were resuspended in pre-warmed assay medium (DMEM (Dulbecco's Modified Eagle's Medium; CORNING, Cat #: 10-013-CV), 10% (v/v) heat-inactivated FBS (Gibco, Cat #: A31606-01), and 100× Penicillin-Streptomycin Solution (CORNING, Cat #: 30-002-CI)). 180 μL of HEK-Blue™ IL-21 cells were incubated with 20 μL of serially diluted heterodimeric Fc-fused IL-21 and its variants in 96-well TC-treated microplates (CORNING, Cat #: 3599) at 37° C. for 22 hours. After incubation, 20 μL of induced HEK-Blue™ IL-21 cell supernatant was incubated with 180 μL of resuspended QUANTI-Blue™ Solution (InvivoGen, Cat #: rep-qbs2) in a flat-bottom 96-well plate (Paul Bottger, Cat #: 05-031-0100) at 37° C. for 2-3 hours. The SEAP (secreted alkaline phosphatase) activity was measured using a spectrophotometer (Varioskan™ LUK, Thermo Scientific, type3020) at 630 nm.

The EC50 value of each heterodimeric Fc-fused IL-21 variants are shown in the table below.

TABLE 3

| | Heterodimeric | Heterodimeric | Heterodimeric | Heterodimeric | Heterodimeric |
|---|---|---|---|---|---|
| | Fc-IL21-WT | Fc-IL21-C01 | Fc-IL21-C03 | Fc-IL21-C07 | Fc-IL21-C08 |
| EC50 | 0.066 | 0.057 | 0.052 | 0.054 | 0.056 |
| (nM) | | | | | |

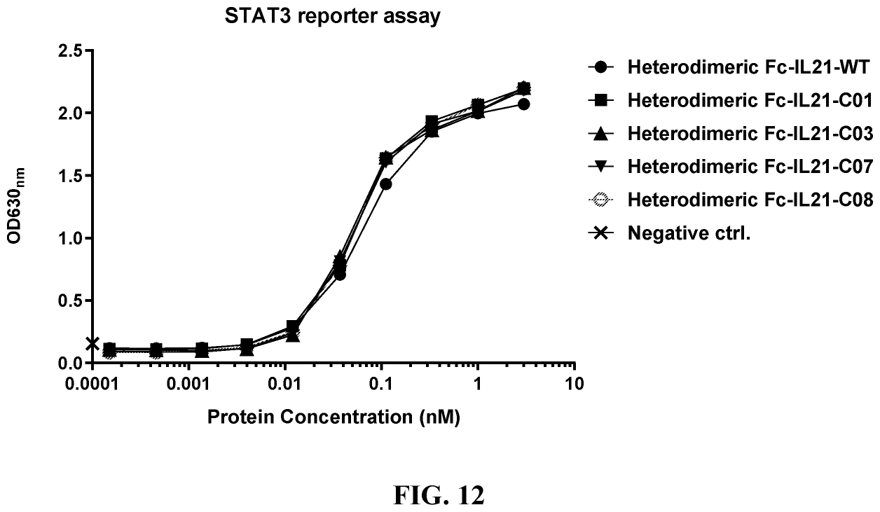

As shown in FIG. 12 and the above table, heterodimeric Fc-IL21-C01, C03, C07 and C08 showed a similar IL-21-STAT3 signaling-inducing activity as compared to heterodimeric Fc-IL21-WT.

### Example 14. Determination of Potency of Heterodimeric Fc-Fused IL-21 Variants on Induction of T Cell and NK-92 Cell Proliferation

For T cell proliferation assays, CD8⁺ T cells were isolated from a hPBMC donor according to manufacturing protocols. The CD8⁺ T cells were activated by CD3/CD28

Dynabeads® (Thermo Fisher, Cat #: 11132D) at a 4:1 cell-to-beads ratio for 2 days. Activated T cells were rested for 24 hours, and 5×10⁴ cells/well of CD8⁺ T cells were then incubated with heterodimeric Fc-fused IL-21 and its variants at indicated concentrations (200 nM, 5-fold serial dilutions, 10 spots), in the presence of 1 μg/ml of soluble anti-CD3 antibody (BioLegend, Cat #: 317347) for 5 days. After the incubation, T cell proliferation was determined by the CellTiter-Glo® Luminescent Cell Viability Assay (Promega, Cat #: G7573). For NK-92 cell proliferation assays, NK-92 cells were starved in a medium without IL-2 supplement for 24 hours. After the starvation, 5×10⁴ cells/well of NK-92 cells were incubated with heterodimeric Fc-fused IL-21 or its variants at indicated concentrations (200 nM, 5-fold serial dilutions, 8 spots) in complete medium (75% MEM-α with ribo- and deoxyribonucleosides (Gibco, Cat #: 12571-048)+12.5% horse serum (Gibco, Cat #: 16050-122)+12.5% FBS (Gibco, Cat #: A31606-01)+150 U/mL IL-2 (BioLegend, Cat #: 589108)) for another 24 hours. The proliferation was determined by the CellTiter-Glo® Luminescent Cell Viability Assay.

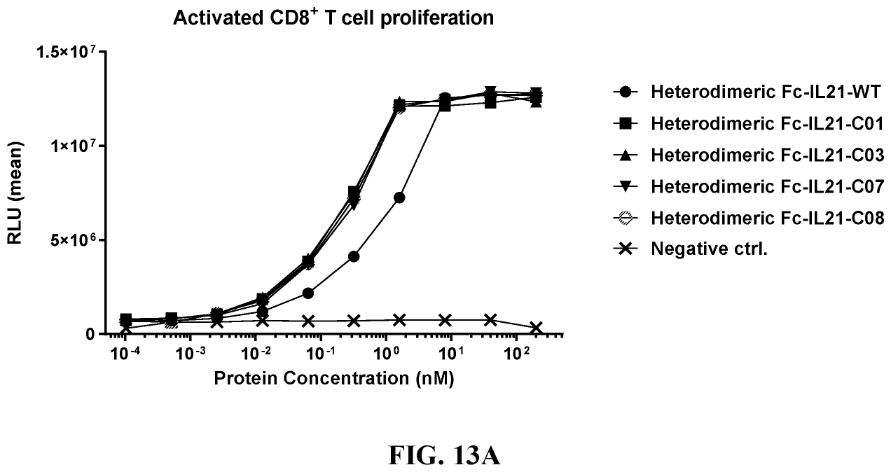

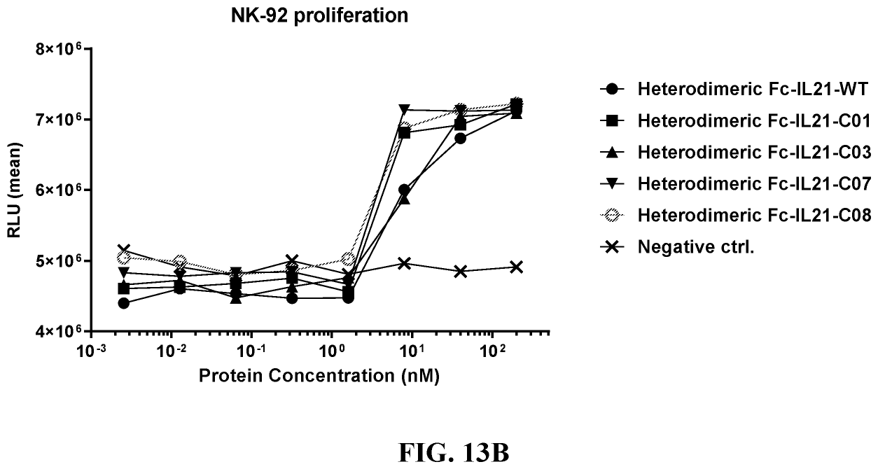

As shown in FIG. 13A and the table below, heterodimeric Fc-IL21-C01, C03, C07 and C08 showed a relatively stronger potency on induction of activated CD8⁺ T cell proliferation than heterodimeric Fc-IL21-WT.

TABLE 4

| | Heterodimeric | Heterodimeric | Heterodimeric | Heterodimeric | Heterodimeric |
|---|---|---|---|---|---|
| | Fc-IL21-WT | Fc-IL21-C01 | Fc-IL21-C03 | Fc-IL21-C07 | Fc-IL21-C08 |
| EC50 | 1.12 | 0.20 | 0.21 | 0.27 | 0.24 |
| (nM) | | | | | |

As showed in FIG. 13B and the table below, heterodimeric Fc-IL21-C01, C07 and C08 showed a better potency on induction of NK-92 cell proliferation than heterodimeric Fc-IL21-WT and C03.

TABLE 5

| | Heterodimeric | Heterodimeric | Heterodimeric | Heterodimeric | Heterodimeric |
|---|---|---|---|---|---|
| | Fc-IL21-WT | Fc-IL21-C01 | Fc-IL21-C03 | Fc-IL21-C07 | Fc-IL21-C08 |
| EC50 | 6.72 | 6.45 | 7.91 | 6.05 | 4.22 |
| (nM) | | | | | |

### Example 15. Determination of Potency of Heterodimeric Fc-Fused IL-21 Variants on Induction of Primary NK Cell Cytotoxicity

The potency of heterodimeric Fc-fused IL-21 variants on induction of primary NK cell cytotoxicity was determined by flow cytometry analysis. Specifically, primary NK cells were isolated from a hPBMC donor according to manufacturing protocols, and K-562 tumor cells were stained with 5 μM of CellTrace™-Violet dye (ThermoFisher, Cat #: C34557). Effector cells (primary NK cell) and target cells (Violet-labeled K-562) were co-incubated at a cell-to-cell ratio of 8:1 in a 96-well U-type cell culture plate, in the presence of 0.00001-100 nM heterodimeric Fc-fused IL-21 or its variants for 24 hours at 37° C. Afterwards, the cells were harvested and stained with 7-AAD viability staining solution (BioLegend, Cat #: 420404). The NK cell cytotoxicity was determined by the percentage of dead K-562 tumor cells by the following formula: the percentage of (CellTrace-Violet⁺7-AAD⁺ cells)/(CellTrace-Violet⁺ cells)*100.

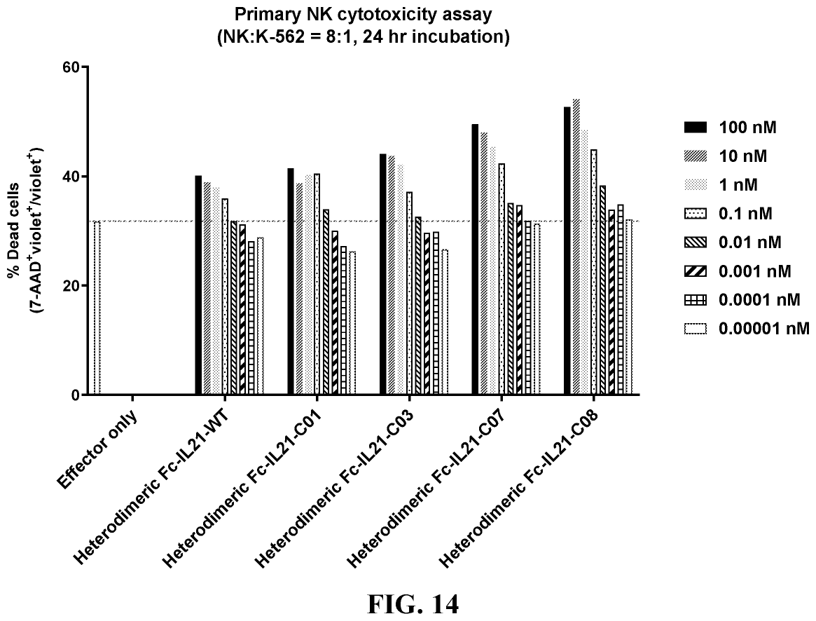

As shown in FIG. 14, heterodimeric Fc-IL21-WT and its variants showed the potency on induction of NK cell cytotoxicity. Heterodimeric Fc-IL21-C07 and C08 showed a slightly stronger potency on induction of primary NK cell cytotoxicity than heterodimeric Fc-IL21-WT and other variants. The results indicate that the selected heterodimeric Fc-IL-21 variants could induce specific lysis of K-562 tumor cells mediated by primary NK cells.

### Example 16. Evaluation of the Antitumor Efficacy of Heterodimeric Fc-Fused IL-21 Variants

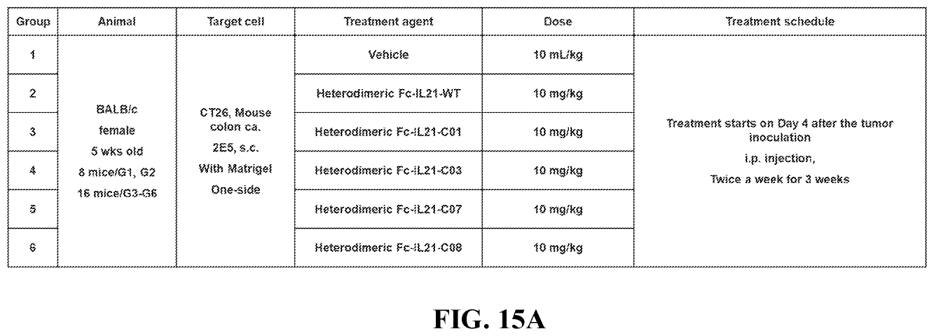

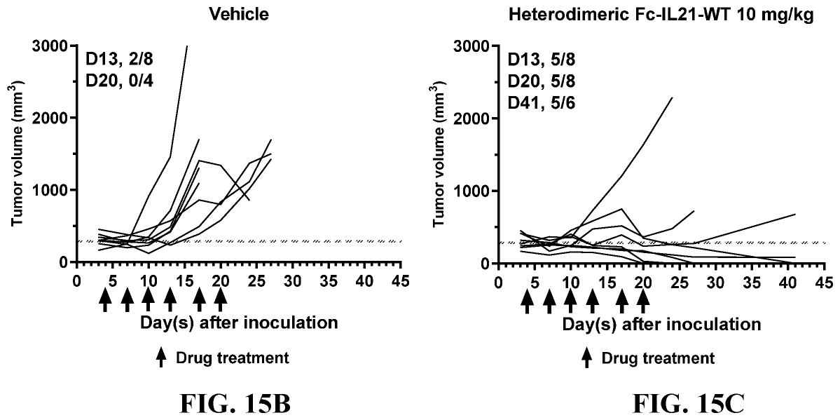

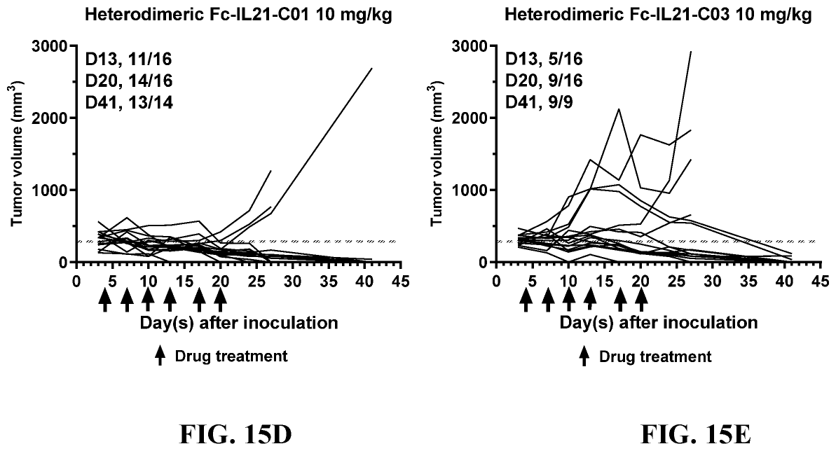

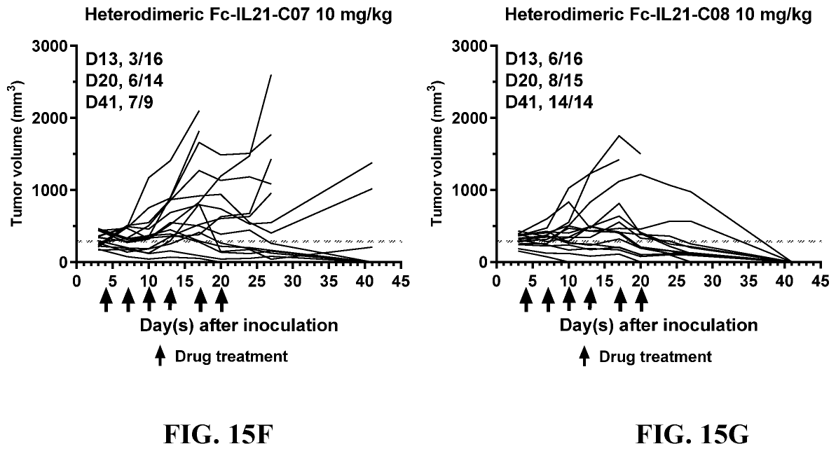

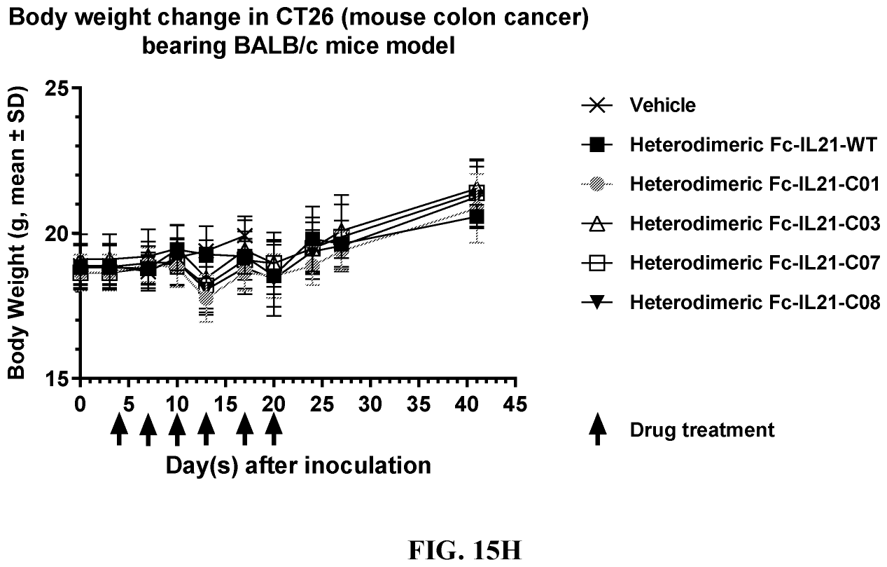

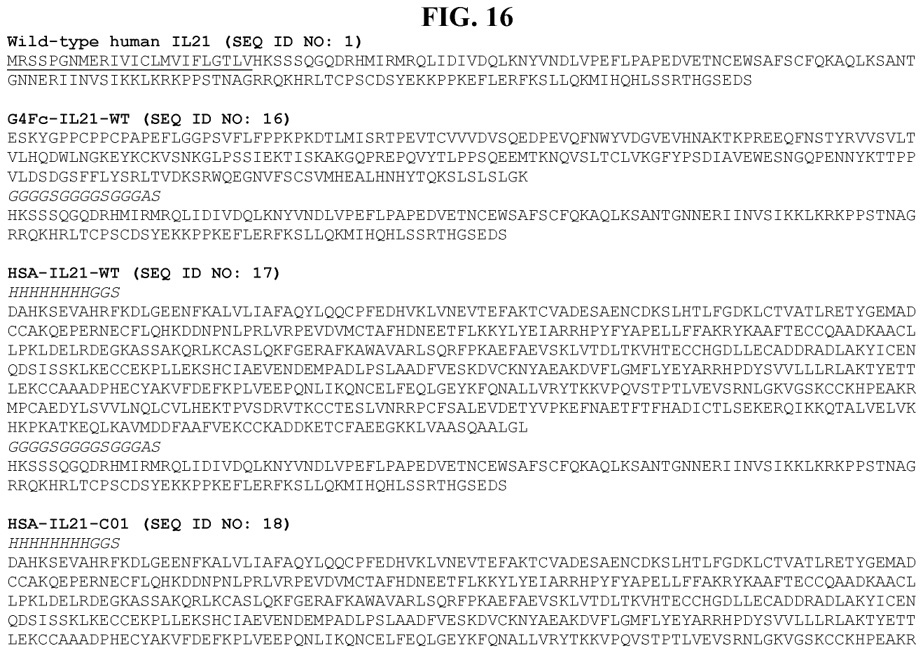

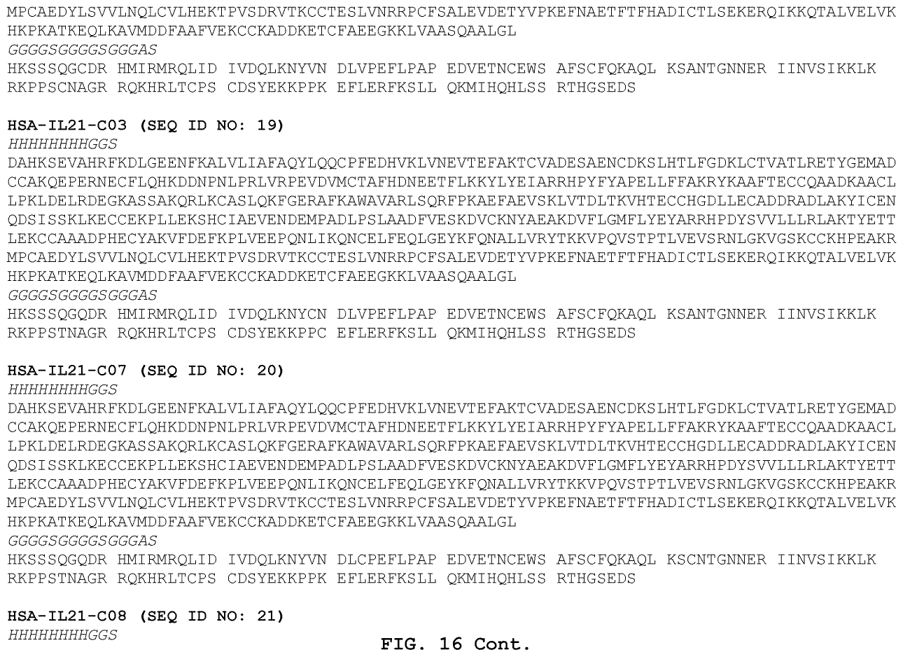

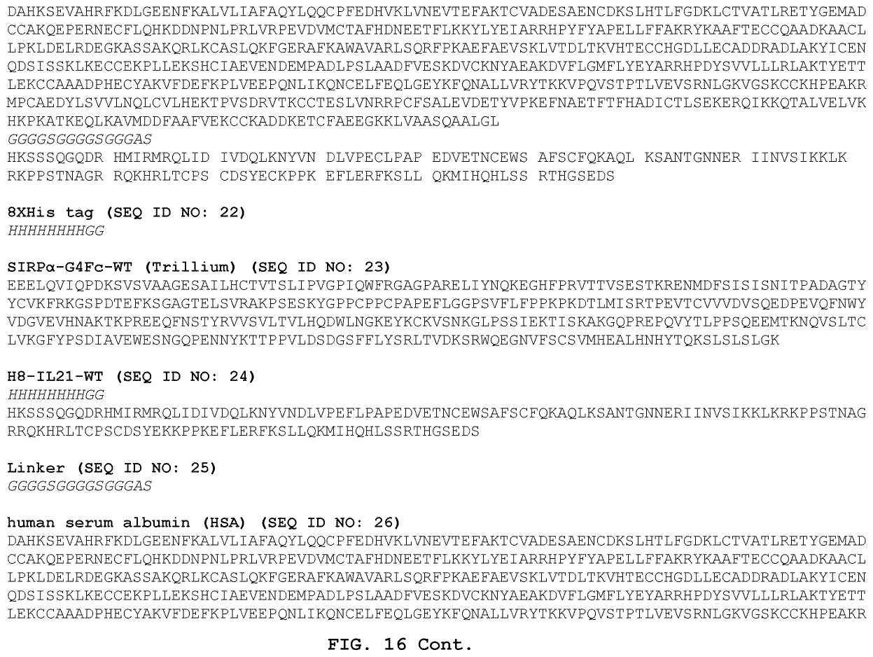

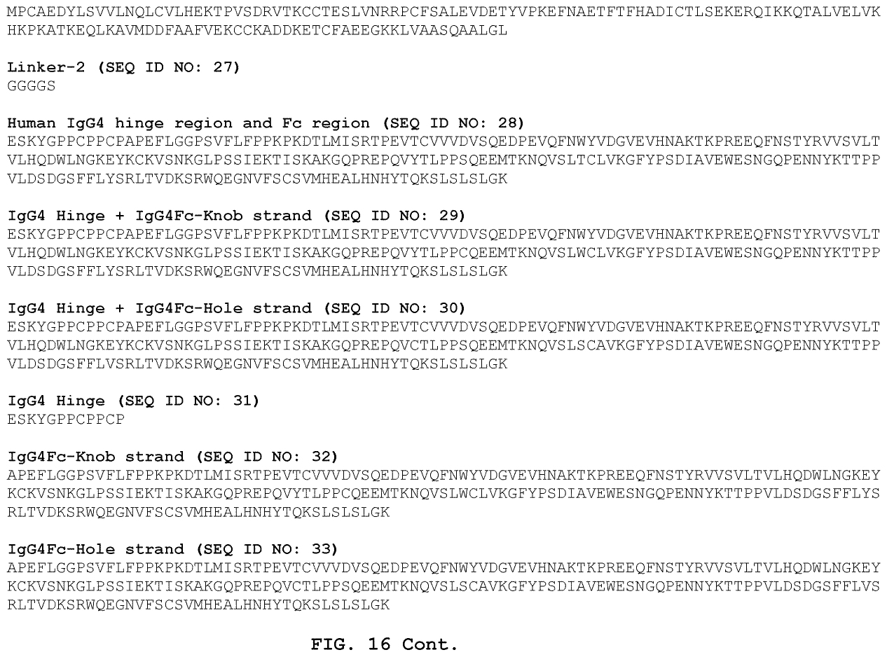

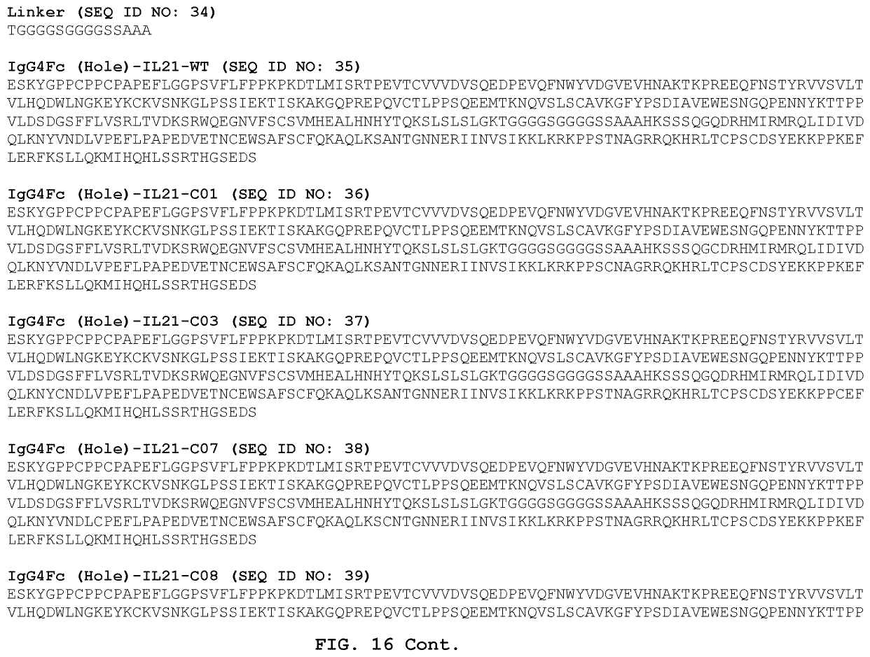

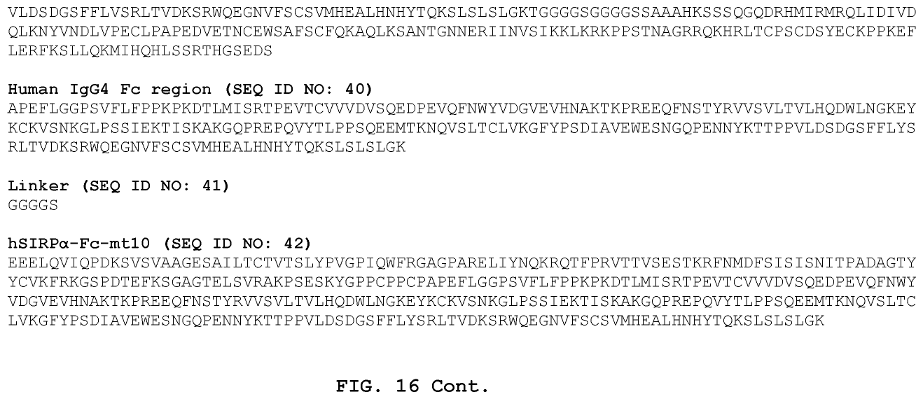

The anti-tumor efficacy of heterodimeric Fc-fused IL-21 and its variants were evaluated in a CT26 tumor-bearing BALB/c mouse model. The treatment plan and dosing schedule are shown in FIG. 15A. Specifically, 5 weeks old BALB/c mice were selected and subcutaneously (s.c.) inoculated with 2× 105 mouse colon cancer cell CT26. The tested molecules treatment started on Day 4 after the tumor inoculation. The treatment group mice were intraperitoneally (i.p.) injected with 10 mg/kg heterodimeric Fc-IL21-WT (G2), heterodimeric Fc-IL21-C01 (G3), heterodimeric Fc-IL21-C03 (G4), heterodimeric Fc-IL21-C07 (G5) and heterodimeric Fc-IL21-C08 (G6), respectively. The control group mice (G1) were injected with 10 mL/kg vehicle (isotonic sodium chloride solution). The injections were performed twice a week (for a total of 3 weeks).

Individual tumor growth curves of each mouse in the G1-G6 groups are shown in FIGS. 15B-15G, respectively. In these figures, the dashed line indicates half the value of the mean tumor volume of the vehicle group (G1) on Day 13, which was 286 mm³. The ratios after “D13,” “D20,” and “D41” show the number of mice with a tumor size under 286 mm³ on Day 13, Day 20, and Day 41 after tumor inoculation, respectively, relative to the total number of lived mice on the same days. The results indicated that heterodimeric Fc-IL21-C01 significantly inhibited the tumor growth compared to heterodimeric Fc-IL21-WT and other variants. In addition, heterodimeric Fc-IL21-C01, C03, C08 induced remarkable tumor shrinkage in this CT26 syngeneic mouse model after the treatment withdrawal (after Day 20). It is possible that heterodimeric Fc-IL21-C01, C03, C08 induced tumor-specific memory responses to inhibit tumor growth. Body weight change results in FIG. 15H suggest that treatment of heterodimeric Fc-fused IL-21 and its variants did not induce significant toxicity on CT26 syngeneic mice.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An engineered IL-21 polypeptide, wherein the engineered IL-21 polypeptide comprises a non-native disulfide bond.

2. The engineered IL-21 polypeptide of claim 1, comprising an amino acid sequence that is at least 80% identical to SEQ ID NO: 2.

3. The engineered IL-21 polypeptide of claim 1 or 2, wherein the engineered IL-21 polypeptide can bind to a complex formed by human IL-21 receptor (IL-21R) and common cytokine γ chain (γc).

4. The engineered IL-21 polypeptide of any one of claims 1-3, wherein the engineered IL-21 polypeptide comprises one or more of the following:
(a) the amino acid that corresponds to position 8 of SEQ ID NO: 2 is cysteine (C);
(b) the amino acid that corresponds to position 19 of SEQ ID NO: 2 is C;
(c) the amino acid that corresponds to position 29 of SEQ ID NO: 2 is C;
(d) the amino acid that corresponds to position 31 of SEQ ID NO: 2 is C;
(e) the amino acid that corresponds to position 33 of SEQ ID NO: 2 is C;
(f) the amino acid that corresponds to position 36 of SEQ ID NO: 2 is C;
(g) the amino acid that corresponds to position 39 of SEQ ID NO: 2 is C; and
(h) the amino acid that corresponds to position 56 of SEQ ID NO: 2 is C.

5. The engineered IL-21 polypeptide of any one of claims 1-4, wherein the engineered IL-21 polypeptide comprises one or more of the following:
(a) the amino acid that corresponds to position 61 of SEQ ID NO: 2 is C;
(b) the amino acid that corresponds to position 62 of SEQ ID NO: 2 is C;
(c) the amino acid that corresponds to position 63 of SEQ ID NO: 2 is C;
(d) the amino acid that corresponds to position 80 of SEQ ID NO: 2 is C;
(e) the amino acid that corresponds to position 86 of SEQ ID NO: 2 is C;
(f) the amino acid that corresponds to position 105 of SEQ ID NO: 2 is C;
(g) the amino acid that corresponds to position 106 of SEQ ID NO: 2 is C;
(h) the amino acid that corresponds to position 107 of SEQ ID NO: 2 is C;
(i) the amino acid that corresponds to position 110 of SEQ ID NO: 2 is C;
(j) the amino acid that corresponds to position 112 of SEQ ID NO: 2 is C; and
(k) the amino acid that corresponds to position 117 of SEQ ID NO: 2 is C.

6. The engineered IL-21 polypeptide of any one of claims 1-5, wherein the engineered IL-21 polypeptide comprises one or more of the following:
(a) the amino acid that corresponds to position 8 of SEQ ID NO: 2 is C, and the amino acid that corresponds to position 86 of SEQ ID NO: 2 is C;
(b) the amino acid that corresponds to position 19 of SEQ ID NO: 2 is C, and the amino acid that corresponds to position 117 of SEQ ID NO: 2 is C;
(c) the amino acid that corresponds to position 29 of SEQ ID NO: 2 is C, and the amino acid that corresponds to position 110 of SEQ ID NO: 2 is C;
(d) the amino acid that corresponds to position 31 of SEQ ID NO: 2 is C, and the amino acid that corresponds to position 62 of SEQ ID NO: 2 is C;
(e) the amino acid that corresponds to position 31 of SEQ ID NO: 2 is C, and the amino acid that corresponds to position 63 of SEQ ID NO: 2 is C;
(f) the amino acid that corresponds to position 33 of SEQ ID NO: 2 is C, and the amino acid that corresponds to position 61 of SEQ ID NO: 2 is C;
(g) the amino acid that corresponds to position 33 of SEQ ID NO: 2 is C, and the amino acid that corresponds to position 63 of SEQ ID NO: 2 is C;
(h) the amino acid that corresponds to position 36 of SEQ ID NO: 2 is C, and the amino acid that corresponds to position 106 of SEQ ID NO: 2 is C;
(i) the amino acid that corresponds to position 36 of SEQ ID NO: 2 is C, and the amino acid that corresponds to position 107 of SEQ ID NO: 2 is C;
(j) the amino acid that corresponds to position 39 of SEQ ID NO: 2 is C, and the amino acid that corresponds to position 105 of SEQ ID NO: 2 is C;
(k) the amino acid that corresponds to position 39 of SEQ ID NO: 2 is C, and the amino acid that corresponds to position 107 of SEQ ID NO: 2 is C;
(l) the amino acid that corresponds to position 39 of SEQ ID NO: 2 is C, and the amino acid that corresponds to position 112 of SEQ ID NO: 2 is C; and
(m) the amino acid that corresponds to position 56 of SEQ ID NO: 2 is C, and the amino acid that corresponds to position 80 of SEQ ID NO: 2 is C.

7. The engineered IL-21 polypeptide of any one of claims 1-6, wherein the engineered IL-21 polypeptide comprises one or more of the following:
(a) the amino acid that corresponds to position 12 of SEQ ID NO: 2 is M;
(b) the amino acid that corresponds to position 16 of SEQ ID NO: 2 is R;
(c) the amino acid that corresponds to position 19 of SEQ ID NO: 2 is I;
(d) the amino acid that corresponds to position 23 of SEQ ID NO: 2 is D;
(e) the amino acid that corresponds to position 105 of SEQ ID NO: 2 is E;
(f) the amino acid that corresponds to position 114 of SEQ ID NO: 2 is E;
(g) the amino acid that corresponds to position 118 of SEQ ID NO: 2 is S;
(h) the amino acid that corresponds to position 121 of SEQ ID NO: 2 is Q;
(i) the amino acid that corresponds to position 122 of SEQ ID NO: 2 is K;
(j) the amino acid that corresponds to position 124 of SEQ ID NO: 2 is I;
(k) the amino acid that corresponds to position 125 of SEQ ID NO: 2 is H; and
(l) the amino acid that corresponds to position 128 of SEQ ID NO: 2 is L.

8. The engineered IL-21 polypeptide of any one of claims 1-7, comprising an amino acid sequence that is at least 85%, 90%, 95%, 96%, 97%, 98%, 99%, or 100% identical to SEQ ID NO: 3, 4, 5, 6, 7, 8, 9, 10, 11, 12, 13, 14, or 15.

9. The engineered IL-21 polypeptide of claim 8, wherein the engineered IL-21 polypeptide comprises an amino acid sequence that is at least 90% identical to SEQ ID NO: 3.

10. The engineered IL-21 polypeptide of claim 8, wherein the engineered IL-21 polypeptide comprises an amino acid sequence that is at least 90% identical to SEQ ID NO: 5.

11. The engineered IL-21 polypeptide of claim 8, wherein the engineered IL-21 polypeptide comprises an amino acid sequence that is at least 90% identical to SEQ ID NO: 9.

12. The engineered IL-21 polypeptide of claim 8, wherein the engineered IL-21 polypeptide comprises an amino acid sequence that is at least 90% identical to SEQ ID NO: 10.

13. The engineered IL-21 polypeptide of any one of claims 1-12, wherein the engineered IL-21 polypeptide can induce proliferation of immune cells (e.g., T cells or NK cells).

14. The engineered IL-21 polypeptide of any one of claims 1-13, wherein the engineered IL-21 polypeptide can induce STAT-3 phosphorylation.

15. A fusion protein comprising the engineered IL-21 polypeptide of any one of claims 1-14.

16. The fusion protein of claim 15, further comprising a human serum albumin (HSA).

17. The fusion protein of claim 16, wherein the HSA comprises an amino acid sequence that is at least 85%, 90%, 95%, 96%, 97%, 98%, 99%, or 100% identical to SEQ ID NO: 26.

18. The fusion protein of claim 16 or 17, wherein the engineered IL-21 polypeptide is linked to the C-terminus to the HSA via a linker peptide.

19. The fusion protein of claim 18, wherein the linker peptide comprises an amino acid sequence that is at least 85%, 90%, 95%, 96%, 97%, 98%, 99%, or 100% identical to SEQ ID NO: 25.

20. The fusion protein of any one of claims 15-19, wherein the fusion protein further comprises a His-tag, optionally at the N-terminus.

21. The fusion protein of any one of claims 15-20, wherein the fusion protein comprises an amino acid sequence that is at least 85%, 90%, 95%, 96%, 97%, 98%, 99%, or 100% identical to SEQ ID NO: 17, 18, 19, 20, or 21.

22. A fusion protein comprising, optionally from N-terminus to C-terminus:
(a) optionally a His-tag;
(b) a HSA;
(c) a linker peptide, and
(d) an engineered IL-21 polypeptide.

23. The fusion protein of claim 22, wherein the His-tag comprises an amino acid sequence that is at least 85%, 90%, 95%, 96%, 97%, 98%, 99%, or 100% identical to SEQ ID NO: 22; wherein the HSA comprises an amino acid sequence that is at least 85%, 90%, 95%, 96%, 97%, 98%, 99%, or 100% identical to SEQ ID NO: 26; wherein the linker peptide comprises an amino acid sequence that is at least 85%, 90%, 95%, 96%, 97%, 98%, 99%, or 100% identical to SEQ ID NO: 25; and/or wherein the engineered IL-21 polypeptide comprises an amino acid sequence that is at least 85%, 90%, 95%, 96%, 97%, 98%, 99%, or 100% identical to SEQ ID NO: 2, 3, 4, 5, 6, 7, 8, 9, 10, 11, 12, 13, 14, or 15.

24. A fusion protein of any one of claims 15-23, further comprising an Fc region.

25. The fusion protein of any one of claims 15-24, wherein the fusion protein can relieve Treg-mediated T cell suppression.

26. The fusion protein of any one of claims 15-25, wherein the fusion protein can induce primary NK cell cytotoxicity.

27. A protein complex comprising:
(a) a first polypeptide comprising from N-terminus to C-terminus: an optional first hinge region, a first Fc region, an optional linker peptide, and the engineered IL-21 polypeptide of any one of claims 1-14; and
(b) a second polypeptide comprising from N-terminus to C-terminus, an optional second hinge region, a second Fc region.

28. The protein complex of claim 27, wherein the first hinge region, the first Fc region, the second hinge region, and/or the second Fc region are derived from human IgG4.

29. The protein complex of claim 27 or 28, wherein the first Fc region and/or the second Fc region comprise one or more knob-into-hole (KIH) mutations.

30. The protein complex of any one of claims 27-29, wherein the first Fc region comprises a sequence that is at least 80%, 90%, 95%, or 100% to SEQ ID NO: 33, and the second Fc region comprises a sequence that is at least 80%, 90%, 95%, or 100% to SEQ ID NO: 32.

31. The protein complex of any one of claims 27-30, wherein the first hinge region and/or the second hinge region comprise a sequence that is at least 80%, 90%, 95%, or 100% to SEQ ID NO: 31.

32. The protein complex of any one of claims 27-31, wherein the linker peptide comprises a sequence that is at least 80%, 90%, 95%, or 100% to SEQ ID NO: 34.

33. The protein complex of any one of claims 27-32, wherein:
(1) the first polypeptide comprises a sequence that is at least 80%, 90%, 95%, or 100% to SEQ ID NO: 35, and the first polypeptide comprises a sequence that is at least 80%, 90%, 95%, or 100% to SEQ ID NO: 29;
(2) the first polypeptide comprises a sequence that is at least 80%, 90%, 95%, or 100% to SEQ ID NO: 36, and the first polypeptide comprises a sequence that is at least 80%, 90%, 95%, or 100% to SEQ ID NO: 29;
(3) the first polypeptide comprises a sequence that is at least 80%, 90%, 95%, or 100% to SEQ ID NO: 37, and the first polypeptide comprises a sequence that is at least 80%, 90%, 95%, or 100% to SEQ ID NO: 29;
(4) the first polypeptide comprises a sequence that is at least 80%, 90%, 95%, or 100% to SEQ ID NO: 38, and the first polypeptide comprises a sequence that is at least 80%, 90%, 95%, or 100% to SEQ ID NO: 29; or
(5) the first polypeptide comprises a sequence that is at least 80%, 90%, 95%, or 100% to SEQ ID NO: 39, and the first polypeptide comprises a sequence that is at least 80%, 90%, 95%, or 100% to SEQ ID NO: 29.

34. The protein complex of any one of claims 27-33, wherein the protein complex can induce STAT-3 phosphorylation and/or relieve Treg-mediated T cell suppression.

35. The protein complex of any one of claims 27-34, wherein the protein complex can induce proliferation of immune cells (e.g., T cells or NK cells), and/or induce primary NK cell cytotoxicity.

36. A pharmaceutical composition comprising the engineered IL-21 polypeptide of any one of claims 1-14, the fusion protein of any one of claims 15-26, or the protein complex of any one of 27-35; and a pharmaceutically acceptable carrier.

37. A nucleic acid encoding the engineered IL-21 polypeptide of any one of claims 1-14, the fusion protein of any one of claims 15-26, or the protein complex of any one of 27-35.

38. A vector comprising the nucleic acid of claim 37.

39. A cell comprising the nucleic acid of claim 37 or the vector of claim 38.

40. The cell of claim 39, wherein the cell is a Expi293 cell or CHO-S cell.

41. A method of producing an engineered IL-21 polypeptide or a fusion protein comprising the engineered IL-21 polypeptide, the method comprising
(a) culturing the cell of claim 39 or 40 under conditions sufficient for the cell to produce the engineered IL-21 polypeptide or the fusion protein; and
(b) collecting the engineered IL-21 polypeptide, the fusion protein, or the protein complex produced by the cell.

42. A method of treating a subject having cancer, the method comprising administering a therapeutically effective amount of a composition comprising the engineered IL-21 polypeptide of any one of claims 1-14, the fusion protein of any one of claims 15-26, or the protein complex of any one of claims 27-35 to the subject.

43. The method of claim 42, wherein the subject has a solid tumor or a hematologic cancer.

44. The method of claim 42, wherein the cancer is melanoma, renal cell carcinoma (RCC), lymphoma, esophageal adenocarcinoma, lung cancer, hepatocellular carcinoma, cholangiocarcinoma, gallbladder cancer, gastric cancer, pancreatic cancer, colorectal cancer, endometrial carcinoma, ovarian cancer, bladder cancer, or prostate cancer.

45. A method of decreasing the rate of tumor growth, the method comprising contacting a tumor cell with an effective amount of a composition comprising the engineered IL-21 polypeptide of any one of claims 1-14, the fusion protein of any one of claims 15-26, or the protein complex of any one of claims 27-35.

46. A method of killing a tumor cell, the method comprising contacting a tumor cell with an effective amount of a composition comprising the engineered IL-21 polypeptide of any one of claims 1-14, the fusion protein of any one of claims 15-26, or the protein complex of any one of claims 27-35.

47. A method of improving the stability of a protein (e.g., cytokine), comprising
(a) providing a 3D structure of the protein (e.g., cytokine),
(b) measuring distance of the Cₐₗₚₕₐ atoms of one or more of amino acid residues in the 3D structure; and
(c) selecting two amino acid residues from the one or more amino acid residues, wherein the Cₐₗₚₕₐ atoms of the two selected amino acid residues are within 3-7 angstroms.

48. The method of claim 47, further comprising expressing a protein variant (e.g., a cytokine variant), wherein the protein variant comprises a non-native disulfide bond formed by mutating the two selected amino acid residues to cysteines.

49. The method of claim 47 or 48, wherein the mutating the two selected amino acid residues to cysteines does not substantially change the 3D structure of the protein (e.g., cytokine).

50. The method of any one of claims 47-49, wherein the protein has no more than 200 amino acid residues.

51. A method of screening a cytokine variant with an improved anti-tumor efficacy, comprising
(a) providing a 3D structure of the cytokine,
(b) measuring distance of the Cₐₗₚₕₐ atoms of one or more of amino acid residues in the 3D structure; and
(c) selecting two amino acid residues from the one or more amino acid residues, wherein the Cₐₗₚₕₐ atoms of the two selected amino acid residues are within 3-7 angstroms.

52. The method of claim 51, further comprising
(d) expressing a cytokine variant, wherein the variant comprises a non-native disulfide bond formed by mutating the two selected amino acid residues to cysteines;
(e) administering the cytokine variant to a tumor-bearing animal; and
(f) determining tumor growth (e.g., by measuring tumor volume) in the tumor-bearing animal.

53. The method of any one of claims 47-52, wherein the protein or cytokine is IL-21 (e.g., human IL-21).


SEQUENCE LISTING (42 sequences)

SEQ ID NO: 1 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 2 - protein, 138 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 3 - protein, 138 aa, synthetic construct
HKSSSQGCDRHMIRMRQLIDIVDQLKNYVNDLVPEFLPAPEDVETNCEWSAFSCFQKAQLKSANTGNNERIINVSIKKLKRKPPSCNAGRRQKHRLTCPSCDSYEKKPPKEFLERFKSLLQKMIHQHLSSRTHGSEDS

SEQ ID NO: 4 - protein, 138 aa, synthetic construct
HKSSSQGQDRHMIRMRQLCDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFCSLLQKMIHQHLSSRTHGSEDS

SEQ ID NO: 5 - protein, 138 aa, synthetic construct
HKSSSQGQDRHMIRMRQLIDIVDQLKNYCNDLVPEFLPAPEDVETNCEWSAFSCFQKAQLKSANTGNNERIINVSIKKLKRKPPSTNAGRRQKHRLTCPSCDSYEKKPPCEFLERFKSLLQKMIHQHLSSRTHGSEDS

SEQ ID NO: 6 - protein, 138 aa, synthetic construct
HKSSSQGQDRHMIRMRQLIDIVDQLKNYVNCLVPEFLPAPEDVETNCEWSAFSCFQKAQLKCANTGNNERIINVSIKKLKRKPPSTNAGRRQKHRLTCPSCDSYEKKPPKEFLERFKSLLQKMIHQHLSSRTHGSEDS

SEQ ID NO: 7 - protein, 138 aa, synthetic construct
HKSSSQGQDRHMIRMRQLIDIVDQLKNYVNCLVPEFLPAPEDVETNCEWSAFSCFQKAQLKSCNTGNNERIINVSIKKLKRKPPSTNAGRRQKHRLTCPSCDSYEKKPPKEFLERFKSLLQKMIHQHLSSRTHGSEDS

SEQ ID NO: 8 - protein, 138 aa, synthetic construct
HKSSSQGQDRHMIRMRQLIDIVDQLKNYVNDLCPEFLPAPEDVETNCEWSAFSCFQKAQLCSANTGNNERIINVSIKKLKRKPPSTNAGRRQKHRLTCPSCDSYEKKPPKEFLERFKSLLQKMIHQHLSSRTHGSEDS

SEQ ID NO: 9 - protein, 138 aa, synthetic construct
HKSSSQGQDRHMIRMRQLIDIVDQLKNYVNDLCPEFLPAPEDVETNCEWSAFSCFQKAQLKSCNTGNNERIINVSIKKLKRKPPSTNAGRRQKHRLTCPSCDSYEKKPPKEFLERFKSLLQKMIHQHLSSRTHGSEDS

SEQ ID NO: 10 - protein, 138 aa, synthetic construct
HKSSSQGQDRHMIRMRQLIDIVDQLKNYVNDLVPECLPAPEDVETNCEWSAFSCFQKAQLKSANTGNNERIINVSIKKLKRKPPSTNAGRRQKHRLTCPSCDSYECKPPKEFLERFKSLLQKMIHQHLSSRTHGSEDS

SEQ ID NO: 11 - protein, 138 aa, synthetic construct
HKSSSQGQDRHMIRMRQLIDIVDQLKNYVNDLVPECLPAPEDVETNCEWSAFSCFQKAQLKSANTGNNERIINVSIKKLKRKPPSTNAGRRQKHRLTCPSCDSYEKCPPKEFLERFKSLLQKMIHQHLSSRTHGSEDS

SEQ ID NO: 12 - protein, 138 aa, synthetic construct
HKSSSQGQDRHMIRMRQLIDIVDQLKNYVNDLVPEFLPCPEDVETNCEWSAFSCFQKAQLKSANTGNNERIINVSIKKLKRKPPSTNAGRRQKHRLTCPSCDSYCKKPPKEFLERFKSLLQKMIHQHLSSRTHGSEDS

SEQ ID NO: 13 - protein, 138 aa, synthetic construct
HKSSSQGQDRHMIRMRQLIDIVDQLKNYVNDLVPEFLPCPEDVETNCEWSAFSCFQKAQLKSANTGNNERIINVSIKKLKRKPPSTNAGRRQKHRLTCPSCDSYEKCPPKEFLERFKSLLQKMIHQHLSSRTHGSEDS

SEQ ID NO: 14 - protein, 138 aa, synthetic construct
HKSSSQGQDRHMIRMRQLIDIVDQLKNYVNDLVPEFLPCPEDVETNCEWSAFSCFQKAQLKSANTGNNERIINVSIKKLKRKPPSTNAGRRQKHRLTCPSCDSYEKKPPKECLERFKSLLQKMIHQHLSSRTHGSEDS

SEQ ID NO: 15 - protein, 138 aa, synthetic construct
HKSSSQGQDRHMIRMRQLIDIVDQLKNYVNDLVPEFLPAPEDVETNCEWSAFSCFCKAQLKSANTGNNERIINVSIKKLCRKPPSTNAGRRQKHRLTCPSCDSYEKKPPKEFLERFKSLLQKMIHQHLSSRTHGSEDS

SEQ ID NO: 16 - protein, 382 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGASHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 17 - protein, 749 aa, synthetic construct
HHHHHHHHGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGASHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 18 - protein, 749 aa, synthetic construct
HHHHHHHHGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGASHKSSSQGCDRHMIRMRQLIDIVDQLKNYVNDLVPEFLPAPEDVETNCEWSAFSCFQKAQLKSANTGNNERIINVSIKKLKRKPPSCNAGRRQKHRLTCPSCDSYEKKPPKEFLERFKSLLQKMIHQHLSSRTHGSEDS

SEQ ID NO: 19 - protein, 749 aa, synthetic construct
HHHHHHHHGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGASHKSSSQGQDRHMIRMRQLIDIVDQLKNYCNDLVPEFLPAPEDVETNCEWSAFSCFQKAQLKSANTGNNERIINVSIKKLKRKPPSTNAGRRQKHRLTCPSCDSYEKKPPCEFLERFKSLLQKMIHQHLSSRTHGSEDS

SEQ ID NO: 20 - protein, 749 aa, synthetic construct
HHHHHHHHGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGASHKSSSQGQDRHMIRMRQLIDIVDQLKNYVNDLCPEFLPAPEDVETNCEWSAFSCFQKAQLKSCNTGNNERIINVSIKKLKRKPPSTNAGRRQKHRLTCPSCDSYEKKPPKEFLERFKSLLQKMIHQHLSSRTHGSEDS

SEQ ID NO: 21 - protein, 749 aa, synthetic construct
HHHHHHHHGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLGGGGSGGGGSGGGASHKSSSQGQDRHMIRMRQLIDIVDQLKNYVNDLVPECLPAPEDVETNCEWSAFSCFQKAQLKSANTGNNERIINVSIKKLKRKPPSTNAGRRQKHRLTCPSCDSYECKPPKEFLERFKSLLQKMIHQHLSSRTHGSEDS

SEQ ID NO: 22 - protein, 10 aa, synthetic construct
HHHHHHHHGG

SEQ ID NO: 23 - protein, 347 aa, synthetic construct
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 148 aa, synthetic construct
HHHHHHHHGGHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 25 - protein, 15 aa, synthetic construct
GGGGSGGGGSGGGAS

SEQ ID NO: 26 - protein, 585 aa, Homo sapiens [sequence omitted]

SEQ ID NO: 27 - protein, 5 aa, synthetic construct
GGGGS

SEQ ID NO: 28 - protein, 229 aa, Homo sapiens
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 31 - protein, 12 aa, Homo sapiens
ESKYGPPCPPCP

SEQ ID NO: 32 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 33 - protein, 217 aa, synthetic construct
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 34 - protein, 15 aa, synthetic construct
TGGGGSGGGGSSAAA

SEQ ID NO: 35 - protein, 382 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGKTGGGGSGGGGSSAAAHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 36 - protein, 382 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGKTGGGGSGGGGSSAAAHKSSSQGCDRHMIRMRQLIDIVDQLKNYVNDLVPEFLPAPEDVETNCEWSAFSCFQKAQLKSANTGNNERIINVSIKKLKRKPPSCNAGRRQKHRLTCPSCDSYEKKPPKEFLERFKSLLQKMIHQHLSSRTHGSEDS

SEQ ID NO: 37 - protein, 382 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGKTGGGGSGGGGSSAAAHKSSSQGQDRHMIRMRQLIDIVDQLKNYCNDLVPEFLPAPEDVETNCEWSAFSCFQKAQLKSANTGNNERIINVSIKKLKRKPPSTNAGRRQKHRLTCPSCDSYEKKPPCEFLERFKSLLQKMIHQHLSSRTHGSEDS

SEQ ID NO: 38 - protein, 382 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGKTGGGGSGGGGSSAAAHKSSSQGQDRHMIRMRQLIDIVDQLKNYVNDLCPEFLPAPEDVETNCEWSAFSCFQKAQLKSCNTGNNERIINVSIKKLKRKPPSTNAGRRQKHRLTCPSCDSYEKKPPKEFLERFKSLLQKMIHQHLSSRTHGSEDS

SEQ ID NO: 39 - protein, 381 aa, synthetic construct
SKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGKTGGGGSGGGGSSAAAHKSSSQGQDRHMIRMRQLIDIVDQLKNYVNDLVPECLPAPEDVETNCEWSAFSCFQKAQLKSANTGNNERIINVSIKKLKRKPPSTNAGRRQKHRLTCPSCDSYECKPPKEFLERFKSLLQKMIHQHLSSRTHGSEDS

SEQ ID NO: 40 - protein, 217 aa, Homo sapiens
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 5 aa, synthetic construct
GGGGS

SEQ ID NO: 42 - protein, 347 aa, synthetic construct
EEELQVIQPDKSVSVAAGESAILTCTVTSLYPVGPIQWFRGAGPARELIYNQKRQTFPRVTTVSESTKRFNMDFSISISNITPADAGTYYCVKFRKGSPDTEFKSGAGTELSVRAKPSESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK